SEQUENCE LISTING (81 sequences)

SEQ ID NO: 1 - DNA, 21 bp, Artificial Sequence
  Description of Artificial Sequence Synthetic primer
tgttccaggtcagtgacttcc

SEQ ID NO: 2 - DNA, 20 bp, Artificial Sequence
  Description of Artificial Sequence Synthetic primer
gcctttttcaacttgcctca

SEQ ID NO: 3 - DNA, 20 bp, Artificial Sequence
  Description of Artificial Sequence Synthetic primer
caacagaaaggaggccaaag

SEQ ID NO: 4 - DNA, 20 bp, Artificial Sequence
  Description of Artificial Sequence Synthetic primer
tcagtaccaagcacgtgagc

SEQ ID NO: 5 - DNA, 24 bp, Artificial Sequence
  Description of Artificial Sequence Synthetic primer
tgcttatggttttatgtaataggc

SEQ ID NO: 6 - DNA, 20 bp, Artificial Sequence
  Description of Artificial Sequence Synthetic primer
tttatgccatggtcctttgg

SEQ ID NO: 7 - DNA, 23 bp, Artificial Sequence
  Description of Artificial Sequence Synthetic primer
aaacatggagctctctgaaaatg

SEQ ID NO: 8 - DNA, 20 bp, Artificial Sequence
  Description of Artificial Sequence Synthetic primer
aggaggtgcaattcagcttt

SEQ ID NO: 9 - DNA, 20 bp, Artificial Sequence
  Description of Artificial Sequence Synthetic primer
gcaattgctgttgaaagctg

SEQ ID NO: 10 - DNA, 20 bp, Artificial Sequence
  Description of Artificial Sequence Synthetic primer
aatgccaaacccatccatta

SEQ ID NO: 11 - DNA, 20 bp, Artificial Sequence
  Description of Artificial Sequence Synthetic primer
tggtccaaatttccatcagc

SEQ ID NO: 12 - DNA, 20 bp, Artificial Sequence
  Description of Artificial Sequence Synthetic primer
tgccaggcatatggttagtg

SEQ ID NO: 13 - DNA, 20 bp, Artificial Sequence
  Description of Artificial Sequence Synthetic primer
atggtttgggggtagaggtc

SEQ ID NO: 14 - DNA, 20 bp, Artificial Sequence
  Description of Artificial Sequence Synthetic primer
ccacaatactgaagggcaca

SEQ ID NO: 15 - DNA, 20 bp, Artificial Sequence
  Description of Artificial Sequence Synthetic primer
cctcaacccagatctctcca

SEQ ID NO: 16 - DNA, 20 bp, Artificial Sequence
  Description of Artificial Sequence Synthetic primer
ggtctcaagcaggtgagctt

SEQ ID NO: 17 - DNA, 22 bp, Artificial Sequence
  Description of Artificial Sequence Synthetic primer
ttctcaaaatctttttggatgc

SEQ ID NO: 18 - DNA, 20 bp, Artificial Sequence
  Description of Artificial Sequence Synthetic primer
gagggactcacaagccacat

SEQ ID NO: 19 - DNA, 20 bp, Artificial Sequence
  Description of Artificial Sequence Synthetic primer
ccttctggccttctgtgaac

SEQ ID NO: 20 - DNA, 20 bp, Artificial Sequence
  Description of Artificial Sequence Synthetic primer
ctcatttggaggcctctgtc

SEQ ID NO: 21 - DNA, 20 bp, Artificial Sequence
  Description of Artificial Sequence Synthetic primer
ccttctgggttctgcttctc

SEQ ID NO: 22 - DNA, 20 bp, Artificial Sequence
  Description of Artificial Sequence Synthetic primer
aactggcctaagggtccact

SEQ ID NO: 23 - DNA, 20 bp, Artificial Sequence
  Description of Artificial Sequence Synthetic primer
ctgagacattgtcccgcttt

SEQ ID NO: 24 - DNA, 20 bp, Artificial Sequence
  Description of Artificial Sequence Synthetic primer
tgtcttcgcacatcttgtcc

SEQ ID NO: 25 - DNA, 20 bp, Artificial Sequence
  Description of Artificial Sequence Synthetic primer
taagccagcttcttccaagc

SEQ ID NO: 26 - DNA, 20 bp, Artificial Sequence
  Description of Artificial Sequence Synthetic primer
gctccagaaccaagaactgc

SEQ ID NO: 27 - DNA, 20 bp, Artificial Sequence
  Description of Artificial Sequence Synthetic primer
aaacagcctgatcccaatgt

SEQ ID NO: 28 - DNA, 20 bp, Artificial Sequence
  Description of Artificial Sequence Synthetic primer
agagtggcccagaaagttca

SEQ ID NO: 29 - DNA, 20 bp, Artificial Sequence
  Description of Artificial Sequence Synthetic primer
ccagctccactcctaacagc

SEQ ID NO: 30 - DNA, 20 bp, Artificial Sequence
  Description of Artificial Sequence Synthetic primer
tggcacacttgtgacaacct

SEQ ID NO: 31 - DNA, 20 bp, Artificial Sequence
  Description of Artificial Sequence Synthetic primer
gatctgatgcttccctggat

SEQ ID NO: 32 - DNA, 20 bp, Artificial Sequence
  Description of Artificial Sequence Synthetic primer
cccctccgacagaacatcta

SEQ ID NO: 33 - DNA, 20 bp, Artificial Sequence
  Description of Artificial Sequence Synthetic primer
ggtgacaaggggtttgaaga

SEQ ID NO: 34 - DNA, 19 bp, Artificial Sequence
  Description of Artificial Sequence Synthetic primer
cctgggcttctaccctgct

SEQ ID NO: 35 - DNA, 19 bp, Artificial Sequence
  Description of Artificial Sequence Synthetic primer
ccaccacagctccagtgag

SEQ ID NO: 36 - DNA, 20 bp, Artificial Sequence
  Description of Artificial Sequence Synthetic primer
tgcttaccctcagcaagaca

SEQ ID NO: 37 - DNA, 20 bp, Artificial Sequence
  Description of Artificial Sequence Synthetic primer
ttctgggtagctcaggctgt

SEQ ID NO: 38 - DNA, 20 bp, Artificial Sequence
  Description of Artificial Sequence Synthetic primer
cacagatgtcagctccgtga

SEQ ID NO: 39 - DNA, 20 bp, Artificial Sequence
  Description of Artificial Sequence Synthetic primer
ttccaactcctccactcagg

SEQ ID NO: 40 - DNA, 20 bp, Artificial Sequence
  Description of Artificial Sequence Synthetic primer
tcattcggccaatacacaga

SEQ ID NO: 41 - DNA, 20 bp, Artificial Sequence
  Description of Artificial Sequence Synthetic primer
gggaaaggacttaggcttgc

SEQ ID NO: 42 - DNA, 21 bp, Artificial Sequence
  Description of Artificial Sequence Synthetic primer
aaatcaactcttgcggctaca

SEQ ID NO: 43 - DNA, 21 bp, Artificial Sequence
  Description of Artificial Sequence Synthetic primer
caacaaaatttggggctaaca

SEQ ID NO: 44 - DNA, 20 bp, Artificial Sequence
  Description of Artificial Sequence Synthetic primer
cactggaaggaggaatttca

SEQ ID NO: 45 - DNA, 21 bp, Artificial Sequence
  Description of Artificial Sequence Synthetic primer
cctttcatgaagcagatgaca

SEQ ID NO: 46 - DNA, 27 bp, Artificial Sequence
  Description of Artificial Sequence Synthetic primer
gctgctacaaatactaagaaatgttcc

SEQ ID NO: 47 - DNA, 24 bp, Artificial Sequence
  Description of Artificial Sequence Synthetic primer
gcttttgatttgttgtctttttga

SEQ ID NO: 48 - DNA, 20 bp, Artificial Sequence
  Description of Artificial Sequence Synthetic primer
cagttggaatgtccatcagc

SEQ ID NO: 49 - DNA, 27 bp, Artificial Sequence
  Description of Artificial Sequence Synthetic primer
tcagctaaagcatattaattcaaaaca

SEQ ID NO: 50 - DNA, 20 bp, Artificial Sequence
  Description of Artificial Sequence Synthetic primer
taggtgggcatgggtgagta

SEQ ID NO: 51 - DNA, 20 bp, Artificial Sequence
  Description of Artificial Sequence Synthetic primer
ccgagcagctggtatttgat

SEQ ID NO: 52 - DNA, 23 bp, Artificial Sequence
  Description of Artificial Sequence Synthetic primer
tgataaatgagaccacgatacca

SEQ ID NO: 53 - DNA, 20 bp, Artificial Sequence
  Description of Artificial Sequence Synthetic primer
cagtgaagagcacacccatt

SEQ ID NO: 54 - DNA, 22 bp, Artificial Sequence
  Description of Artificial Sequence Synthetic primer
ggcattggctagatacacaaaa

SEQ ID NO: 55 - DNA, 21 bp, Artificial Sequence
  Description of Artificial Sequence Synthetic primer
tcattctttgctgtggaaaca

SEQ ID NO: 56 - DNA, 23 bp, Artificial Sequence
  Description of Artificial Sequence Synthetic primer
ccccaaaagtagacaatttcctt

SEQ ID NO: 57 - DNA, 25 bp, Artificial Sequence
  Description of Artificial Sequence Synthetic primer
tggtttgatcaatagtttcttaggg

SEQ ID NO: 58 - DNA, 20 bp, Artificial Sequence
  Description of Artificial Sequence Synthetic primer
cccaagagaggagggagttt

SEQ ID NO: 59 - DNA, 20 bp, Artificial Sequence
  Description of Artificial Sequence Synthetic primer
atctctgatcccaggaggtg

SEQ ID NO: 60 - DNA, 20 bp, Artificial Sequence
  Description of Artificial Sequence Synthetic primer
tgagaatttcatgcgagagc

SEQ ID NO: 61 - DNA, 19 bp, Artificial Sequence
  Description of Artificial Sequence Synthetic primer
cctgggcttctaccctgct

SEQ ID NO: 62 - DNA, 19 bp, Artificial Sequence
  Description of Artificial Sequence Synthetic primer
ccaccacagctccagtgag

SEQ ID NO: 63 - DNA, 20 bp, Artificial Sequence
  Description of Artificial Sequence Synthetic primer
ctcacaaaacctgcccttgt

SEQ ID NO: 64 - DNA, 20 bp, Artificial Sequence
  Description of Artificial Sequence Synthetic primer
gtgggtagctctgctgaagg

SEQ ID NO: 65 - DNA, 21 bp, Artificial Sequence
  Description of Artificial Sequence Synthetic primer
aaggagcaggaactcatcaca

SEQ ID NO: 66 - DNA, 19 bp, Artificial Sequence
  Description of Artificial Sequence Synthetic primer
cctgggcttctaccctgct

SEQ ID NO: 67 - DNA, 19 bp, Artificial Sequence
  Description of Artificial Sequence Synthetic primer
ccaccacagctccagtgac

SEQ ID NO: 68 - DNA, 20 bp, Artificial Sequence
  Description of Artificial Sequence Synthetic primer
cctccgatcgttgtcagaag

SEQ ID NO: 69 - DNA, 19 bp, Artificial Sequence
  Description of Artificial Sequence Synthetic primer
ctggcgtaatagcgaagag

SEQ ID NO: 70 - DNA, 18 bp, Artificial Sequence
  Description of Artificial Sequence Synthetic primer
aagagcaccgtggctgcc

SEQ ID NO: 71 - DNA, 21 bp, Artificial Sequence
  Description of Artificial Sequence Synthetic primer
gtgcaggtaatgatgatcata

SEQ ID NO: 72 - DNA, 20 bp, Artificial Sequence
  Description of Artificial Sequence Synthetic primer
ccaccagcctttcctaaaca

SEQ ID NO: 73 - DNA, 19 bp, Mus sp.
attttcatagccattgtgg

SEQ ID NO: 74 - DNA, 19 bp, Mus sp.
attttcataaccattgtgg

SEQ ID NO: 75 - DNA, 19 bp, Mus sp.
attttcatarccattgtgg

SEQ ID NO: 76 - DNA, 19 bp, Mus sp.
agattagaccttgttcagg

SEQ ID NO: 77 - DNA, 19 bp, Mus sp.
agattagagattgttcagg

SEQ ID NO: 78 - DNA, 19 bp, Mus sp.
agattagacmttgttcagg

SEQ ID NO: 79 - DNA, 19 bp, Mus sp.
atgtcctggatcagctcct

SEQ ID NO: 80 - DNA, 19 bp, Mus sp.
atgtcctgggtcagctcct

SEQ ID NO: 81 - DNA, 19 bp, Mus sp.
atgtcctggrtcagctcct

(19) United States
(12) Patent Application Publication
(10) Pub. No.: US 2010/0069251 A1
(43) Pub. Date: Mar. 18, 2010

(54) METHODS FOR PRODUCING EMBRYONIC STEM CELLS FROM PARTHENOGENETIC EMBRYOS

(75) Inventors: Kitai Kim, Brighton, MA (US); George Daley, Weston, MA (US)

Correspondence Address: DAVID S. RESNICK, NIXON PEABODY LLP, 100 SUMMER STREET, BOSTON, MA, 02110-2131, US

(73) Assignee: CHILDREN'S MEDICAL CENTER CORPORATION, Boston, MA (US)

(21) Appl. No.: 12/440,964

(22) PCT Filed: Sep. 13, 2007
(86) PCT No.: PCT/US2007/019935

§ 371 Date: Mar. 12, 2009

Related U.S. Application Data
(60) Provisional application No. 60/844,769, filed on Sep. 15, 2006.

(51) Int. Cl. C40B 30/00; C12N 5/02; C12Q 1/68; C40B 40/02

(52) U.S. Cl. 506/7; 435/377; 435/6; 506/14

(57) ABSTRACT

Means for producing embryonic stem (pES) cells which have a heterozygous genome that is matched to an individual donor are provided. In one embodiment, a means for the generation and isolation of parthenogenetic embryonic stem (pES) cells which have regions of heterozygosity that are fully matched to the oocyte donor at the MHC loci (e.g. (h-)p(MI)ES cells is provided. This is in contrast to the traditional methods of parthenogenesis that generate parthenogenetic embryonic stem (pES) cells having a substantially homozygous haploidentical set of chromosomes that are homozygous at the MHC loci.

### CROSS REFERENCE TO RELATED APPLICATIONS

This Application claims the benefit under 35 U.S.C. §119(e) of U.S. Provisional Application No. 60/844,769 filed Sep. 15, 2006.

### GOVERNMENT SUPPORT

This invention was made with Government support under grants HL71265 (NIH/NHLB1), DK59279 (NIH/NIDDK), DK70055(NIH), OD000256-01 (NIH Director's Pioneer Award), CA86991 (NIH/NCI), awarded by the National Institute of Health. The government has certain rights to the invention.

### FIELD OF THE INVENTION

The present invention relates to methods for producing parthenogenetic embryonic stem (pES) cells whose genome is heterozygous, i.e. genetically matched to the DNA of a donor. In embodiments of the invention, means for producing and isolating pES cells that carry the full complement of major histocompatibility complex (MHC) antigens of the oocyte donor, e.g. pES cells that are heterozygous at the human leukocyte antigen, are described.

### BACKGROUND

Parthenogenesis entails the development of an embryo directly from an oocyte without fertilization. Many animal and plant species reproduce via parthenogenesis, but mammalian embryonic development requires genomic contributions from both maternal and paternal chromosomes due to the importance in development of genes that are termed “imprinted” because they are expressed differently depending on their inheritance through the maternal or paternal gametes. Mammalian oocytes that are activated to divide without fertilization will develop into embryos that contain only maternally imprinted chromosomes. Because they lack gene expression from paternally-imprinted genes, parthenogenetic embryos develop only to the early limb bud stage in mouse (5). Parthenogenetic ES (pES) cells have been isolated at the blastocyst stage of parthenogenetic development from mice and primates (1, 2). Parthenogenetic ES cells contribute widely to adult tissues in chimeric mice (1) and both mouse and primate pES cells undergo extensive differentiation in vitro (2, 3). A human case of parthenogenetic chimerism has been described in which the hematopoietic system and skin were derived from parthenogenetic cells (6). In addition to pluripotent stem cells from fertilized embryos and embryos created by somatic cell nuclear transfer, parthenogenesis represents another method for creation of pluripotent stem cells that might be used as a source of tissue for transplantation.

In experimental parthenogenesis, oocytes arrested at metaphase II of meiosis (MID are chemically activated in the presence of cytochalasin, a drug that interferes with completion of MII by preventing extrusion of the 2^{nd} polar body. Diploidy is maintained, and the resulting pseudozygote can develop into a blastocyst from which p(MII)ES cells can be isolated. ES cells derived via parthenogenesis contain a duplicated haploid genome and are thus predominantly homozygous (haplo-identical). Because of the reduced number of histocompatibility antigens expressed on p(MII)ES cells, it has been suggested that they might represent a source of transplantable tissues that can be more readily matched to patients, and might pose less risk for tissue rejection.

However, transplantation of homozygous cells matched at only one of two haplotypes of the MHC loci presents several unique immunologic challenges. In heterozygous recipients, homozygous tissues may be subject to rejection by natural killer (NK) cells that recognize the absence of histocompatibility antigens, a phenomenon termed “hybrid resistance” in bone marrow transplantation (7). Another immunologic complication of partially-matched hematopoietic tissue transplants is the phenomenon of transfusion-associated graft-versus host disease, which can result when blood products from rare individuals who are homozygous at the Human Leukocyte Antigen (HLA) loci are transfused into heterozygous recipients who are matched at one of the donor haplotypes. In this case, the recipient's unmatched HLA genes serve as a target for immune attack. This problem has been described most often in the Japanese population, where homozygosity of HLA antigens occurs with appreciable frequency (8, 9). In solid organ transplantation, MHC matched tissues are favored over allogeneic tissues, and even partial MHC antigen matching enhances organ allograft survival (10). The most certain strategy for avoiding immunologic complications is to transplant genetically identical tissues, but this limits transplantation to autologous tissues, transplants between monozygotic twins, or cells created by somatic cell nuclear transfer.

A means for producing embryonic stem cells that are not haplo-identical and that are heterozygous at the Human Leukocyte Antigen (HLA) loci, is highly desirable. Such cells can provide a source for histocompatible tissues for transplantation that are patient specific.

### SUMMARY OF THE INVENTION

Means for producing embryonic stem (pES) cells which have a heterozygous genome that is matched to an individual donor are provided.

In one embodiment, a means for the generation and isolation of parthenogenetic embryonic stem (pES) cells which have regions of heterozygosity that are fully matched to the oocyte donor at the MHC loci ((h-)pES cells) is provided. This is in contrast to the traditional methods of parthenogenesis that generate parthenogenetic embryonic stem (pES) cells having a substantially homozygous haploidentical set of chromosomes that are homozygous at the MHC loci.

In one embodiment, a method for producing a heterozygous embryonic stem (ES) cell line is provided. The method comprises: a) obtaining a diploid oocyte that is in prophase or metaphase I of meiosis I, wherein the diploid oocyte comprises DNA derived from a single individual male or female; b) culturing the oocyte under conditions that inhibit formation of the first polar body such that the cell remains diploid; c) activating the oocyte of step (b) to induce parthenogenetic development; d) culturing said activated oocyte to produce an embryo comprising a discernible trophectoderm and an inner cell mass; e) isolating said inner cell mass, or cells therefrom, and transferring said inner cell mass, or cells, to an in vitro media that inhibits differentiation of said inner cell mass or cells derived therefrom; and f) culturing said inner cell mass cells, or cells derived therefrom, to maintain said cells in an undifferentiated state thereby generating an embryonic stem cell line that is substantially heterozygous. In one embodiment, step (f) further comprises maintaining the cells in a pluripotent state. In one embodiment, the method further includes step (g) that comprises analyzing the cells of step (f) for heterozygosity at a desired locus and selecting cells that are heterozygous at said desired locus.

In one embodiment, the DNA derived from a single individual male or female is human DNA, the desired locus is a Human Leukocyte Antigen (HLA) locus and cells that are heterozygous for at least one HLA locus are selected.

In one embodiment, the cells that are heterozygous for at least one HLA locus are analyzed for diploid or tetraploid DNA content.

In one embodiment, embryonic stem cells that have diploid DNA content are selected and maintained in a pluripotent state.

In one embodiment, embryonic stem cells that have tetraploid DNA content are selected and maintained in a pluripotent state.

In one embodiment, the HLA locus is selected from the group consisting of: HLA-A, HLA-B, HLA-C, HLA-DR, HLA-DQ, and HLA-DP.

In one embodiment, the cells that are heterozygous for at least one HLA locus are heterozygous at each of the following HLA loci: HLA-A, HLA-B, HLA-C, HLA-DR, HLA-DQ, and HLA-DP.

In one embodiment, the diploid oocyte is a human, non-human primate, murine, bovine, porcine, or ovine.

In one embodiment, the diploid DNA derived from a single individual is human, bovine, primate, murine, ovine, or porcine.

In one embodiment, the diploid ocyte is gynogenetically produced.

In one embodiment, the diploid ocyte is androgenetically produced.

In one embodiment, the conditions that inhibit formation of the first polar body include incubation of said oocyte with cytochalasin D.

In one embodiment, the diploid cells are human oocytes containing human male or human female DNA.

In one embodiment, the cultured cells are allowed to differentiate.

In one embodiment, the cultured cells are differentiated into hematopoietic stem cells.

In one embodiment, the cultured cells are implanted at a desired site in vivo that is to be engrafted with cells or tissue. In one embodiment, the cells are implanted in an immunocompromised non-human animal. In one embodiment, the site is a wound, a joint, muscle, bone, or the central nervous system.

In one embodiment, the cell obtained by step (f) is genetically modified.

Also provided is a method for producing stem cells that are heterozygous for at least one MHC locus. The method comprises: a) obtaining oocyte cells in metaphase II that comprises haploid DNA derived from a single individual male or female, which optionally may be genetically modified; b) activating the oocyte cells of step (b) to induce parthenogenetic development under conditions that inhibit second polar body formation;

c) culturing said activated oocytes to produce an embryos comprising a discernible trophectoderm and an inner cell mass; d) isolating said inner cell mass, or cells therefrom, and transferring said inner cell mass, or cells, to an in vitro media that inhibits differentiation of said inner cell mass or cells derived therefrom thereby generating pluripotent embryonic stem (pES) cell lines; and e) selecting pES cell lines that have undergone recombination at least one MHC locus; and f) culturing the pES cells of step (e) to maintain said cells in an undifferentiated state thereby generating a pES cell line that is heterozygous for at least one MHC locus.

In one embodiment, the pES cell line of step (f) that is heterozygous for at least one MHC locus comprises human DNA and is heterozygous at a Human Leukocyte Antigen (HLA) locus selected from the group consisting of HLA-A, HLA-B, HLA-C, HLA-DR, HLA-DQ, and HLA-DP.

In one embodiment, the pES cell line is heterozygous at each of the following Human Leukocyte Antigen (HLA) loci: HLA-A, HLA-B, HLA-C, HLA-DR, HLA-DQ, and HLA-DP.

In one embodiment, step (f) further comprises maintaining the cells in a pluripotent state.

In one embodiment, the cells of step (f) are analyzed for diploid or tetraploid DNA content.

In one embodiment, the embryonic stem cells that have diploid DNA content are selected and maintained in a pluripotent state.

In one embodiment, the embryonic stem cells that have tetraploid DNA content are selected and maintained in a pluripotent state.

In one embodiment, the oocyte cells are human, non-human primate, murine, bovine, porcine, or ovine.

In one embodiment, the DNA derived from a single individual is human, bovine, primate, murine, ovine, or porcine.

In one embodiment, the oocyte cells in metaphase II are gynogenetically or androgenetically produced.

In one embodiment, the conditions that inhibit formation of the second polar body comprise incubation of the oocyte with cytochalasin B.

In one embodiment, the oocytes are human oocytes comprising human male or human female DNA.

In one embodiment, the cells of (f) are allowed to differentiate.

In one embodiment, the cells of (f) are implanted at a desired site in vivo that is to be engrafted with cells or tissue. In one embodiment, the cells are implanted in an immunocompromised non-human animal. In one embodiment, the site is a wound, a joint, muscle, bone, or the central nervous system.

In one embodiment, the cells obtained by (f) are genetically modified.

A stem cell bank comprising a library or plurality of human or non-human animal embryonic stem cell lines generated by the methods described herein is also provided. In one embodiment the stem cell bank comprises h-p(MI)ES cells (cells derived from a parthogenesis embryo wherein first polor body formation was inhibited). In one embodiment the bank comprises h-p(MII)ES cells.

In one embodiment, the cultured cells are differentiated into hematopoietic stem cells.

Also provided is a method for determining if an embryonic stem cell line was derived from either i) a parthenogenesis embryo wherein first polor body formation was inhibited (a (pMI)ES cell line), ii) a parthenogenesis embryo wherein second polor body formation was inhibited (a (pMII)ES cell line), iii) a nuclear transfer embryo (a ntES cell line), or iv) a natural fertilization embryo comprising the steps of: a) genotyping the cells for heterozygosity using heterozygous SNP markers b) plotting the heterozygous rate (heterozygous SNP markers/total SNP makers) versus SNP marker distance from centromere on a graph wherein the X axis is the heterozygous rate and the Y axis is the SNP marker distance from centromere; and c) obtaining a slope from the graph of step b wherein a negative slope in step (c) indicates a p(MI)ES cell line; a positive slope in step (c) indicates a p(MII)ES cell line; and no discernable slope in step (c) indicates a ntES cell line or a cell line derived from a natural fertilization embryo.

### BRIEF DESCRIPTION OF THE DRAWINGS

FIGS. 1a to 1c show diagrammatic representation of chromosome dynamics during normal and artificial oocyte maturation. FIG. 1a, Normal oocyte maturation and fertilization. Immature oocytes arrested in the diplotene stage of the first meiotic prophase harbor 40 paired homologous chromosomes (20 bivalents, also called tetrads). A single bivalent is shown on the left. Recombination commences during the preceding pachytene phase. Hormonal stimulation promotes oocyte maturation, at which point the bivalents separate, the crossovers resolve, and either the maternal or paternal copy of each homologous chromosome pair segregate into the first polar body (1st PB), completing meiosis I (MI). Further oocyte maturation results in arrest at meiosis II (MII) until fertilization occurs. At fertilization the oocyte is activated, the centromeres split, and half the chromosomes are extruded via the second polar body (2nd PB). The incoming sperm nucleus restores the diploid chromosome complement, and mitotic cleavage ensues. Blastocysts derived by fertilization yield fES cells. Recombination is not illustrated here. FIG. 1b, Parthenogenetic oocyte maturation. During experimental parthenogenesis, the MII arrested oocytes are activated by chemical treatment (alcohol or calcium ionophore), but extrusion of the 2nd polar body is inhibited by cytochalasin B (CCB), yielding a duplicated largely-haploid genome that develops into an embryo from which parthenogenetic ES cells are isolated. Recombination events result in h-p(MII)ES cells. FIG. 1c, Oocyte maturation with blockade of MI. Inhibiting extrusion of the first polar body followed by further oocyte maturation results in a reduction to diploidy with the extrusion of a polar body. Substantial genetic identity with the oocyte donor is maintained, except for regions of recombination, which retain haplo-identity. Recombination rates appear suppressed because of the probability for co-segregation of recombinant chromosomes into the same cell. Given that recombination is in fact observed in most lines, we call ES cells isolated in this manner h-p(MI)ES cells.

FIGS. 2a and 2b show polymorphism studies on the H2K gene of the Major Histocompatibility Complex (MHC) from C57BL/6×CBA F1 pES cells. FIG. 2a, Schematic of PCR amplicon from H2K locus with BsiEI restriction enzyme site polymorphism present in C57BL/6 (B6). FIG. 2b, Genotyping of genomic DNA samples by digestion of the PCR amplicon from the H2K region with the BsiEI restriction enzyme (12). Lane 1: 100 bp size marker; lane 2: uncut PCR product; lane 3: uncut spiked DNA (internal control for BsiEI restriction digestion); lane 4: C57BL/6, digested with BsiEI; lane 5: CBA, incubated with BsiEI but not digested; lanes 6-14: nine p(MII)ES cells from B6CBAF1 mice incubated with BsiEI; lanes 15-19: five p(MI)ES cells from B6CBAF1 mice incubated with BsiEI. As internal controls for the completion of restriction enzyme digestion, samples 6-19 were spiked with a DNA fragment containing a BsiEI restriction enzyme site. The parthenogenetic ES lines were derived on mouse embryonic feeder (MEF) cells derived from CD1 mice carrying a unique mutation of the tyrosinase gene. Absence of MEF contamination was confirmed by tyrosinase PCR and mutation specific restriction enzyme digestion (data not shown) (25). Black (closed) circles mark two h-p(MII)ES cells that have retained both maternal H2K genes. White (open) circles represent 5 h-p(MI)ES cells. The amount of undigested (CBA) fragment exceeds the digested (B6) PCR fragment because of the inefficient restriction enzyme digestion of the heteroduplexes formed between the B6 and CBA alleles during PCR amplification. FIG. 2c, H2K protein expression on differentiated p(MII)ES/h-p(MII)ES/h-p(MI)ES cells. Cells were differentiated for 14-15 days as embryoid bodies (EBs), dissociated with collagenase, and plated on gelatin-coated tissue culture dishes in EB differentiation media (26) for an additional 15 days. The resulting populations of epithelial cells were stained with fluorescent antibodies against the H2 Kb (C57BL/6) and H2Kk (CBA) proteins and analyzed by flow cytometry for surface expression.

FIG. 3 shows sequencing based SNP analysis of peri-centromeric markers on each mouse chromosome. FIG. 3, Location of peri-centromeric SNP markers. The sequencing results demonstrated detection of different SNP signals for homozygous C57BL/6 and CBA, as well as heterozygous B6CBAF1.

FIGS. 4a to 4h show genotyping analysis of single nucleotide polymorphisms (SNPs) in p(MII)ES/p(MI)ES cells to determine peri-centromeric homozygosity or heterozygosity. Strain specific SNP signals for C57BL/6, CBA, or B6CBAF1 were detected by sequencing of a PCR amplicon that harbored a strain-specific SNP. Loci for which the SNP allele was detected are marked beneath the relevant strain according to legend. Chromosome number of the SNP is indicated. Genomic DNA samples from C57BL/6 and CBA mice were tested as controls (FIG. 4a, FIG. 4b). Genomic DNA from one h-p(MII)ES cell line (FIG. 4c) and five h-p(MI)ES cell lines (FIG. 4d-FIG. 4h) were tested at SNPs located within 3.4-9.9 Mbps of the centromere.

FIGS. 5a to 5c show graphic representations of recombination in p(MII)ES/p(MI)ES/fertilized embryo derived ES (fES) cells detected by genotyping of chromosome 17 SNPs. Upper panels of 5a-5c: ES cell lines were examined for C57BL/6 and CBA strain-specific SNP signals at 3.4 Mbp, 32.0 Mbp, and 60.5 Mbp from the centromere on chromosome 17. Dark grey bar indicates a heterozygous genotype; dotted bar indicates homozygous. FIG. 5a, p(MII)ES; FIG. 5b, p(MI)ES; FIG. 5c, fES cells. Bottom panels of 5a-5c: The heterozygosity at each locus, calculated as frequency of heterozygosity/total number of cell lines genotyped, was plotted against the SNP marker distance from the centromere of chromosome 17.

FIGS. 6a to 6c show graphs of genotype analysis of the NT-1 cell line demonstrates its status as a p(MII)ES cell. FIG. 6a, The DNA finger print data on the NT-1 cell line from the Seoul National University (SNUIC) were plotted according to the heterozygous status vs. gene distance from the centromere. Marker in underlined letter indicates homozygosity while the letter without underline indicates heterozygosity. There is a clear predominance of peri-centromeric homozygosity. FIG. 6b, Graphical depiction of the relationship between heterozygosity and marker distance from the centromere enables a determination of the provenance of p(MII)ES/p(MI)ES/nt/ or fES cells. FIG. 6c, Data on NT-1 cell line plotted as heterozygosity (y value) vs. average marker gene distance from centromere (x value). Peri-centromeric homozygosity and a rising slope of heterozygosity of markers at increasingly telomeric markers are diagnostic of a p(MII)ES cell.

FIGS. 7a to 7c shows genome-wide SNP genotyping analysis for representative p(MII)ES, p(MI)ES, and pan-heterozygous (polyploid)-p(MI)EScells. Left panels: Depiction of genotypes for each chromosome, from centromere (cen, top) to telomere (tel, bottom), revealing blocks, or haplotypes of markers, indicative of crossing-over events prior to isolation of pES cells. Markers that show homozygosity of the C57BL/6 SNP are light gray; homozygous CBA SNPs are white; and a heterozygous genotype is indicated in dark gray. FIG. 7a, p(MII)ES; FIG. 7b, p(MI)ES; FIG. 7c, pan-heterozygous p(MI)ES cells. Genotyping was performed at the Broad Institute NCRR Center for Genotyping and Analysis using the Illumina multiplexed allele extension and ligation method (Golden Gate) with detection using oligonucleotide probes covalently attached to beads which are assembled into fiber optic bundles (Bead Array) (27, 28). Right panels: results of genotyping of all cells of a given type (n=17 for p(MII)ES; n=12 for p(MI)ES; and n=8 for pan-heterozygous p(MI)ES) are plotted as the heterozygous rate (heterozygous SNP markers/total SNP makers) vs. SNP marker distance from centromere. A positive slope is indicative of a p(MII)ES cell; a negative slope indicates a p(MI)ES cell; and universal heterozygosity indicates a fES cell derived from an F1 mating of two inbred mouse strains, or alternatively, a pan-heterozygous (polyploidy) p(MI)ES cell of the F1 mouse.

FIGS. 8a to 8b shows a schematic of varied recombination events that can occur in parthogenesis when first polar body formation is inhibited (Allelic segregation during MI parthenogenesis.) In this protocol, the first meiotic division is inhibited by cytochalasin D followed by chemical activation. The genotyping data on the p(MI)ES cells is then most consistent with independent chromatid segregation during the second meiotic division. This schema demonstrates the possible outcomes of the chromatid segregation process, assuming independent segregation after recombination events on one homologous chromatid pair. FIG. 8a, If crossing over has not occurred at the MHC locus (distal recombination), all 4 genotypes will remain heterozygous at the MHC locus. FIG. 8b, However, if crossing over has occurred at the MHC (proximal recombination) and chromatids that exchanged DNA during crossing over do not segregate together, homozygosity at the locus will be maintained (HOMr). Alternatively, if the chromatids that exchanged DNA during crossing over at the MHC do segregate together, heterozygosity at the locus will be restored (HET/HETr). For simplicity, recombination involving only one of the two sister chromatids is shown. In fact, both sisters of paired homologues may undergo recombination, which will yield a more complex pattern of genotypes, but all tending to favor maintenance of heterozygosity. Different parental origins of homologous chromosomes are represented by distinct colors. The MHC locus is represented by circles.

FIGS. 9a to 9b show polymorphism studies on Tap1 gene from C57BL/6×CBA F1 h-p(MI)ES. FIG. 9a, Schematic of PCR amplified region of Tap1 gene on chromosome 17. HhaI restriction enzyme site is absent in C57BL/6, but present in CBA strain. FIG. 9b, Genotyping by HhaI digestion of Tap1 gene PCR amplicon. The Tap1 gene is only 0.05 cM away from H2K gene, but whereas the H2K allele PCR product is digested by BsiEI in C57BL/6, but not in CBA, the Tap1 gene PCR product is digested by HhaI in CBA, but not in C57BL/6. Using this reverse pattern of PCR product digestion, we demonstrated that all h-p(MI)ES lines were heterozygous for the polymorphism at the Tap1 locus—a gene neighboring the MHC locus. Lane 1: 100 bp size marker; lane 2: uncut PCR product; lane 3: uncut spiked DNA; lane 4: spiked DNA, HhaI digested; lane 5: C57BL/6 incubated with HhaI but not digested; lane 6: CBA HhaI digested; lane 7: B6CBAF1 incubated with HhaI, note that both fragments are present; lane 8-13: seven h-p(MI)ES cells from B6CBAF1 mice incubated with HhaI and showing both fragments, confirming heterozygosity at this locus; lane 14: 100 bp size marker. As internal controls for complete restriction enzyme digestion, samples 5-13 were spiked with a DNA fragment containing HhaI restriction enzyme sites (see lanes 3 and 4).

FIG. 10 shows a Southern blot analysis to determine the methylation status of the imprinted Rasgrf1 locus in p(MI)ES cells. Genomic DNA of 16 p(MI)ES cell clones and controls were digested with PstI/NotI, and hybridized with a probe from the Rasgrf1 gene, as described (29). This locus is typically methylated on the paternal allele, which renders it resistant to restriction digestion, thereby yielding a fragment length of 8 kbp. The unmethylated maternal allele results in a 3 kbp fragment. Digestion of parthenogenetic ES cells (p(MII)ES) shows the maternal allele. Digestion of androgenetic ES cells (aES) that are derived from reconstruction of a zygote with 2 male pronuclei shows only the paternal alleles, while ES cells isolated from fertilized embryos (fES) shows both. All p(MI)ES cell clones reveal the maternal pattern.

FIGS. 11a to 11b show graphs of the hematopoietic developmental potential of p(MII)ES/h-p(MII)ES/h-p(MI)ES cells. FIG. 11a, Flow cytometry on day 6 EB-derived cells. p(MII)ES/h-p(MII)ES/h-p(MI)ES/fES cells were stained with relevant antibodies to detect CD41+, CD41+ckit-high+, and CD45+ hematopoietic cells. All ES cells showed equivalent primitive hematopoietic populations. FIG. 11b, Formation of myeloid colonies in methylcellulose supplemented with hematopoietic cytokines (M3434; Stem Cell Technologies). Colony numbers are per 100,000 cells from day 6 EBs. Robust hematopoietic colonies, displaying a similar contribution of all myeloid lineages can be observed in all ES cell lines.

FIGS. 12a to 12c show fluorescence-activated cell-sorting (FACS) of transplanted p(I)ES cells and p(II)ES cells. FIG. 12a, negative control; FIG. 12b sorted hematopoietic stem cells (HSC) from p(I)ES cells; FIG. 12c sorted hematopoietic stem cells (HSC) from p(II)ES cells. Green fluorescent protein (GFP) positive cells indicate that blood cells differentiated from the transplanted p(II)ES cells or p(I)ES cells are present in peripherial blood.

FIGS. 13a to 13 c show patterns of genomic homozygosity and heterozygosity in ES cells derived by nuclear transfer (nt) and parthenogenesis from F1 hybrid mice. (FIG. 13a) Schematic of chromosomal genotypes predicted for ES cells of indicated types. Heterozygous region (HET); Homozygous region (HOM). (FIG. 13b) Depiction of SNP genotypes of a representative clone of male ntES cells and female p(MII)ES cells. Chromosome numbers are indicated along the top, and markers are arrayed for the acrocentric murine chromosomes from Centromeric (Cen; top) to Telomeric (Tel; bottom) in blocks that span a physical distance of 2 Mbp. Distance is marked in megabase pairs (Mbp). Light grey blocks: homozygous (HOM) haplotypes; dark grey blocks: heterozygous (HET) haplotypes. (FIG. 13c) Graphs show the heterozygosity of SNP markers plotted against SNP marker distance from the centromere. N=30 for ntES; n=5 for p(MII). Slope function describing the data is indicated.

FIGS. 14a to 14b show SNP genotype data for SCNT-hES-1 and three representative human ES cell lines. Genome-wide SNP mapping was performed using the GeneChip Human Mapping 500K SNP Array. (FIG. 14a) SCNT-hES-1. Genotyping data is depicted as in FIG. 1, except that short p arm of the human chromosomes project superiorly, while long q arm projects inferiorly. Note peri-centromeric regions of homozygosity for each chromosome. Conversion to homozygosity near telomeres is a reflection of the high frequency of double recombination in human chromosomes; (FIG. 14b) Genotyping data for three human ES lines (H9, BGO1, and BG03) generated from fertilization embryos. The patterns of pan-heterozygosity were identical for all three lines (excepting the X chromosome data, which shows homozygosity in the male line BG-01); thus the data is presented as a composite. Light grey blocks: homozygous (HOM) haplotypes; dark grey blocks: heterozygous (HET) haplotypes. (FIG. 14c) Heterozygosity of SNP markers plotted against SNP marker distance from the centromere for the four cell lines. Slope function is indicated.

FIGS. 15a to 15c show bisulphite sequencing of three differentially methylated regions (DMRs) in SCNT-hES-1 cells. Circles represent the position and methylation status of individual CpG sites (filled, methylated; open, unmethylated) and each line represents a unique clone of DNA. The numbering of the first and last CpG sites for H19 (FIG. 15a) and SNRPN (FIG. 15c) DMRs are relative to the transcriptional start sites shown, and the numbering for KCNQ1OT1 DMR (FIG. 15b) is according to the KCNQ1 sequence (AJ006345). A polymorphism in the KCNQ1OT1 DMR distinguished the two alleles (lines indicated by square and circle).

FIGS. 16a to 16h show Genome-wide SNP genotyping of ntEScells. The Panels show genotypes for each chromosome, from centromere(cen, top) totelomere (tel, bottom), revealing blocks, or haplotypes, of markers. Light grey blocks: homozygous (HOM) SNP regions; dark grey blocks: heterozygous (HET) SNP. (FIG. 16a) LN1 (B cell nt-donor cells from C57BL/6N×DBA/2J F₁) ¹; (FIG. 16b) LN2 (T cell nt-donor cells from C57BL/6N×129 svjae F₁) ¹; (FIG. 16c) V6.5 NSC B1 (neuronal stem cell nt-donor cells from C57BL/6N×129 svjae F₁) ²; (FIG. 16d) ESCC cells (fibroblast nt-donor cells form C57BL/6N×M.cast F₁) ¹; (FIG. 16e) BCT-1F (fibroblast nt-donor cells from C57BL/6N×C3H/HeJF₁) ³; (FIG. 16f) BCC-5 (cumulus nt-donor cells from C57BL/6N×C3H/HeJF₁) ³; (FIG. 16g) BDC-2, BDC-5, BDC-9, BDC-10, BDC-11, and BDC-13 (cumulus nt-donor cells from C57BL/6N×DBA/2J) BDT-1F (fibroblast nt-donor cells from C57BL/6N×DBA/2J). BCC-1, BCC-3, BCC-4, and BCC-6 (cumulus nt-donor cells from C57BL/6N×C3H/HeJ F₁) ³; (FIG. 16h) LN3 (T cell nt-donor cells from C57BL/6N×129 svjae F₁) ¹. V6.5 NSC B2 (neuronal stem cell nt-donor cells from C57BL/6N×129 svjae F₁) ². BDT-2, BDT-3, BDT-5, BDT-6, BDT-7, and BDT-8 (fibroblastnt-donor cells from C57BL/6N×DBA/2J F1). BCT-1, BCT-2, BCT-3, BCT-4, and BCT-5 (fibroblastnt-donor cells from C57BL/6N×C3H/HeJF₁) ³. Superscript 1 refers to (Brambrink et al., 2006), Superscript 2 refers to (Blelloch et al., 2006), Superscript 3 refers to (Wakayama et al., 2006).

FIGS. 17a to 17c show SNP genotyping of human ES cell lines BGO3, H9, BG01, and SCNT-hES-1. Panels depict results of SNP genotyping data for each chromosome indicated, from centromere (cen) to telomere (p arm, top half; q arm, bottom half). Blue lines indicate indicative heterozygous SNP markers. HOM: homozygous regions (reflected in <5% frequency of heterozygous SNPs); HET: heterozygous SNP regions. 2000, 4000, and 6000 show the number of the SNP markers from thecentromere. (FIG. 17a) X chromosome control for heterozygosity; BGO3 and H9 are predominantly heterozygous female lines with two X chromosomes. BGO1 control for assigning homozygosity due to the hemizygous X-chromosome (genotyping error rate of 2.3%); SCNT-hES-1 data is consistent with similar hemizygosity of the X chromosome. (FIG. 17b) chromosome 10; A typical pericentromeric homozygosity can be observed only in SCNT-hES-1 (FIG. 17c) chromosome 6 p-arm. The green arrow indicates the location of the MHC (human HLA antigen) cluster. The MHC cluster is located on the border of a homozygous region indicating that the cross-over event occurred telomeric to the MHC-gene cluster.

FIG. 18 shows a table depicting DNA finger print analysis of SCNT-hES-1.

### DETAILED DESCRIPTION OF THE INVENTION

The present invention is directed to methods for producing embryonic stem cells via parthenogeneis. In particular, methods are described for producing embryonic stem cells that are substantially heterozygous, i.e genetically matched to the oocyte donor, e.g. genetically matched at MHC loci.

As used herein, a “stem cell” is a cell that has the ability to proliferate in culture, producing some daughter cells that remain relatively undifferentiated, and other daughter cells that give rise to cells of one or more specialized cell types; and “differentiation” refers to a progressive, transforming process whereby a cell acquires the biochemical and morphological properties necessary to perform its specialized functions. Stem cells therefore reside immediately antecedent to the branch points of the developmental tree.

As used herein, an “embryonic stem (ES) cell line” is a cell line with the characteristics of the murine embryonic stem cells isolated from morulae or blastocyst inner cell masses (as reported by Martin, G., Proc. Natl. Acad. Sci. USA (1981) 78:7634-7638; and Evans, M. and Kaufman, M., Nature (1981) 292: 154-156); i.e., ES cells are capable of proliferating indefinitely and can differentiate into all of the specialized cell types of an organism, including the three embryonic germ layers, all somatic cell lineages, and the germ line. ES cells have high nuclear-to-cytoplasm ratio, prominent nucleoli, are capable of proliferating indefinitely and can be differentiate into most or all of the specialized cell types of an organism, such as the three embryonic germ layers, all somatic cell lineages, and the germ line. ES cells that can differentiate into all of the specialized cell types of an organism are totipotent. In some cases, ES cells are obtained that can differentiate into almost all of the specialized cell types of an organism; but not into one or a small number of specific cell types. For example, Thomson et al. describe isolating a primate ES cell that, when transferred into another blastocyst, does not contribute to the germ line (Proc. Natl. Acad. Sci. USA. (1995) 92:7844-7848). Such ES cells are an example of stem cells that are nearly totipotent.

The term “embryonic stem cell line” is intended to include embryonic stem-like cells (ES-like cell) which are cell lines isolated from an animal inner cell mass or epiblast that has a flattened morphology, prominent nucleoli, is immortal, and is capable of differentiating into all somatic cell lineages, but when transferred into another blastocyst typically does not contribute to the germ line. An example is the primate “ES cell” reported by Thomson et al. (Proc. Natl. Acad. Sci. USA. (1995) 92:7844-7848). The term “embryonic stem cell line” is also intended to include “inner cell mass-derived cells” (ICM-derived cells) are cells directly derived from isolated ICMs or morulae without passaging them to establish a continuous ES or ES-like cell line. Methods for making and using ICM-derived cells are described in U.S. Pat. No. 6,235,970, the contents of which are incorporated herein in their entirety.

As used herein, a “totipotent” cell is a stem cell with the “total power” to differentiate into any cell type in the body, including the germ line following exposure to stimuli like that normally occurring in development. An example of such a cell is an ES cell, an embryonic germ cell, an ICM-derived cell, or a cultured cell from the epiblast of a late-stage blastocyst.

As used herein, a “nearly totipotent cell” is a stem cell with the power to differentiate into most or nearly all cell types in the body following exposure to stimuli like that normally occurring in development. An example of such a cell is an ES-like cell.

As used herein, “pluripotent cell” refers to a cell derived from an embryo produced by activation of a cell containing DNA of all female or male origin that can be maintained in vitro for prolonged, theoretically indefinite period of time in an undifferentiated state that can give rise to different differentiated tissue types, i.e., ectoderm, mesoderm, and endoderm. This would include by way of example, but not limited to, mesenchymal stem cells that can differentiate into bone, cartilage and muscle; hemotopoietic stem cells that can differentiate into blood, endothelium, and myocardium; neuronal stem cells that can differentiate into neurons and glia; and so on. In one embodiment, the pluripotent state of said cells is maintained by culturing inner cell mass or cells derived from the inner cell mass of an embryo produced by androgenetic/parthenogenetic methods under appropriate conditions, e.g. by culturing on a fibroblast feeder layer or another feeder layer or culture that includes leukemia inhibitory factor. The pluripotent state of such cultured cells can be confirmed by various methods known in the art, e.g., (i) confirming the expression of markers characteristic of pluripotent cells; (ii) production of chimeric animals that contain cells that express the genotype of said pluripotent cells; (iii) injection of cells into animals, e.g., SCID mice, with the production of different differentiated cell types in vivo; and (iv) observation of the differentiation of said cells (e.g., when cultured in the absence of feeder layer or LIF) into embryoid bodies and other differentiated cell types in vitro.

As used herein, “parthenogenesis” refers to the process by which activation of the oocyte (female gamete) occurs in the absence of sperm (male gamete) penetration. Parthenogenesis refers to the development of an early stage embryo comprising trophectoderm and inner cell mass that is obtained by activation of an oocyte, comprising DNA of all female or all male origin. “parthenogenetic embryos” refers to an embryo that only contains all female chromosomal DNA that is derived from female gametes. For example, parthenogenetic embryos can be derived by activation of unfertilized female gametes, e.g., unfertilized human, rabbit, bovine, or murine oocytes. Parthenogenetic embryos can also be derived from androgenesis.

As use herein, “DNA derived from an individual male or female” refers to DNA derived from a mammalian male gamete or from a mammalian female gamete. The DNA may optionally be genetically modified. In one embodiment, the mammal is human.

As used herein, “androgenesis” refers to the production of an embryo containing a discernible trophectoderm and inner cell mass that results upon activation of an oocyte or other embryonic cell type, e.g. blastomere, that contains, DNA of all male origin, e.g., human spermatozoal DNA. Optionally, said DNA of all male origin may be genetically modified, e.g., by the addition, deletion, or substitution of at least one DNA sequence. Methods for generating androgenic embryos by reconstitution with two sperm nuclei are described in U.S. Patent Application publication 2003/0129745. An oocyte or blastomere cell that is “androgenetically produced” refers to an oocyte or blastomere cell that has been reconstituted with two sperm nuclei.

As used herein, the term “gynogenesis” refers to the production of an embryo containing a discernible trophectoderm and inner cell mass that results upon activation of a cell, preferably an oocyte, or other embryonic cell type, containing mammalian DNA of all female origin. In one embodiment, the DNA is of human female origin, e.g., human or non-human primate oocyte DNA. Such female mammalian DNA may be genetically modified, e.g., by insertion, deletion or substitution of at least one DNA sequence, or may be unmodified. For example, the DNA may be modified by the insertion or deletion of desired coding sequences, or sequences that promote or inhibit embryogenesis. Typically, such embryo will be obtained by in vitro activation of an oocyte that contains DNA of all female origin. “Gynogenesis” is inclusive of parthenogenesis of a female gamete which is defined above. It also includes activation methods wherein the sperm or a factor derived therefrom initiates or participates in activation, but the spermatozoal DNA does not contribute to the DNA in the activated oocyte. “Gynogenesis” is also inclusive of pathogenesis activation of an oocyte generated by fusion of two haploid embryos, each containing DNA from a female donor. An oocyte or blastomere cell that is “gynogenetically produced” thus includes an oocyte or blastomere cell that has been reconstituted with two female haploid nuclei.

As used herein, “diploid cell” refers to a cell, e.g., an oocyte or blastomere, having a diploid DNA content. A diploid oocyte has 46 chromosomes in human, one set (23 chromosomes) originating from each parent. As used herein, “diploid DNA” refers to 46 chromosomes, one male and one female set.

As used herein, “haploid cell” refers to a cell, e.g., an oocyte or blastomere having a haploid DNA content, wherein the haploid DNA is of all male or all female origin. As used herein. “haploid DNA” refers to 23 chromosomes of all male or all female origin in human, with the exception of any recombination that may have occurred between male and female chromosomes.

As used herein, “activation” refers to a process wherein fertilized or unfertilized oocyte, undergoes a process typically including separation of the chromatid pairs and extrusion of the second polar body, resulting in an oocyte having a haploid number of chromosomes, each with one chromatid. In one embodiment of the invention, diploid oocytes in prophase I or metaphase I of Meiosis 1 where first polar body formation has been inhibited are activated. In another embodiment, activation is effected under one of the following conditions that inhibit second polar body formation, i.e. (i) conditions that do not cause second polar body extrusion; (ii) conditions that cause polar body extrusion but wherein polar body extrusion is inhibited; or (iii) conditions that inhibit first cell division of haploid oocyte. “Activation” refers to methods whereby a cell containing DNA of all male or female origin is induced to develop into an embryo that has a discernible inner cell mass and trophectoderm, which is useful for producing pluripotent, or totipotent, cells but which is itself incapable of developing into a viable offspring. Embodiments of the invention also include activation of oocytes or blastomere cells that have been transplanted with two male (androgenesis) or two female haploid nuclei (gynogenesis).

As used herein, “Meiosis I” refers to a stage of development wherein in prophase I, homologous chromosomes pair. The paired chromosomes are called bivalents that have two chromosomes and four chromatids, with one chromosome coming from each parent. During Metaphase I, bivalents, each composed of two chromosomes (four chromatids) align at the metaphase plate. The orientation is random, with either parental homologue on a side giving a 50-50 chance for the daughter cells to get either the mother's or father's homologue for each chromosome.

As used herein, “Meiosis II” refers to stage of cell development wherein the DNA content of a cell consists of a haploid number of chromosomes with each chromosome represented by two chromatids.

As used herein, the term “embryo” refers to an embryo that results upon activation of a cell, e.g., oocyte or other embryonic cells containing DNA of all male or all female origin, which optionally may be modified, that comprises a discernible trophectoderm and inner cell mass, which cannot give rise to a viable offspring and wherein the DNA is of all male or female origin. The inner cell mass or cells contained therein are useful for the production of pluripotent cells as defined previously.

As used herein, the term “inner cell mass” refers to the inner portion of an embryo which gives rise to fetal tissues. Herein, these cells are used to provide a continuous source of pluripotent, or totipotent, cells in vitro. In the present invention, the inner cell mass refers to the inner portion of the embryo that results from androgenesis or gynogenesis, i.e., embryos that result upon activation of cells containing DNA of all male or female origin. In one embodiment, the DNA is human DNA, e.g., human oocyte or spermatozoal DNA, which optionally has been genetically modified.

As used herein. The term “trophectoderm” refers to a portion of early stage embryo which gives rise to placental tissues. In the present invention, the trophectoderm is that of an embryo that results from, embryos that result from activation of cells that contain DNA of all male or all female origin, e.g., human oocyte or spermatozoan.

As used herein, the term “Differentiated cell” refers to a non-embryonic cell that possesses a particular differentiated, i.e., non-embryonic state. The three earliest differentiated cell types are endoderm, mesoderm and ectoderm.

As used herein, “ex vivo” cell culture refers to culturing cells outside of the body. Ex vivo cell culture includes cell culture in vitro, e.g., in suspension, or in single- or multi-well plates. Ex vivo culture also includes co-culturing cells with two or more different cell types, and culturing in or on 2- or 3-dimensional supports or matrices, including methods for culturing cells alone or with other cell types to form artificial tissues.

Methods for Producing an Embryonic Cell Line that is Substantially Heterozygous

In one embodiment of the invention, a method for producing an embryonic stem (ES) cell line that is substantially heterozygous (referred to herein as p(MI)ES cells) is provided. The method comprises a) obtaining a diploid oocyte that is in prophase or metaphase I of meiosis I, wherein the diploid oocyte comprises DNA derived from a single individual male or female; b) culturing the oocyte under conditions that inhibit formation of the first polar body such that the cell remains diploid; c) activating the oocyte of step (b) to induce parthenogenetic development; d) culturing said activated oocyte to produce an embryo comprising a discernible trophectoderm and an inner cell mass; e) isolating said inner cell mass, or cells therefrom, and transferring said inner cell mass, or cells, to an in vitro media that inhibits differentiation of said inner cell mass or cells derived therefrom; and f) culturing said inner cell mass cells, or cells derived therefrom, to maintain said cells in an undifferentiated state thereby generating a embryonic stem cell line that is substantially heterozygous.

As used herein, “substantially heterozygous” refers to a non-haplo-identical genome, i.e. a haploid genome of an oocyte in Meiosis II that has not duplicated itself; rather the genome has genetic similarity to the original oocyte diploid DNA of Meiosis I, e.g. original oocyte DNA obtained from the mother. Substantially heterozygous refers to at least 1%, at least 2%, at least 5%, at least 10%, at least 20%, at least 30%, at least 40%, at least 50%, at least 60%, at least 70%, at least 80% or at least 90% genetic identity to the original diploid DNA of the oocyte. Genetic identity can occur at any loci.

As used herein, “substantially heterozygous embryonic stem cell or stem cell line” refers to embryonic stem cells that retain substantial genetic identity with the oocyte donor, i.e. the embryonic stem cell does not contain a duplicated haploid genome of the donor.

The first step to producing an embryonic stem (ES) cell line that is substantially heterozygous is to obtain a mammalian diploid oocyte that is in prophase or metaphase I of meiosis I. Means for collecting oocytes are known to those in the art and include, but is not limited to, superovulation. Various superovulation methods have been designed for humans as well as other mammals, see for example U.S. patent publication application 2003/0232430 for human procedures.

In one embodiment, oocytes are collected from humans after superovulation has been induced with initial treatment of gonadotropins (for example, but not limited to, Serpophene (clomiplene), Gonal-F, Follistin, Repronex, Pergonal or humegon) or with gonadotropin-releasing hormone (GnRH) followed by hormone injection of hCG. In humans, ovulation usually occurs 36-48 hours following the hCG injection and oocytes can be harvested. Multiple ways for superovulattion of mammals (e.g, cattle, sheep, pig, equine) are known in the art and methods can be readily modified thereto.

In one embodiment, oocytes are collected after inducing superovulation with pregnant mare serum gonadotropin (PMSG) followed by injection with human chorionic gonadotropin (hCG). For example, oocytes can be collected at about 3-about 9 hours after hCG injection. To obtain oocytes that are in prophase or metaphase I of meiosis I, cumulus cells can be dispersed by incubation of the cells hyaluronidase (Sigma, H4272: 1 mg/ml in KSOM) and cumulus-free oocytes collected and cultured in suitable media, e.g. KSOM (Specialty Media, MR-106-D).

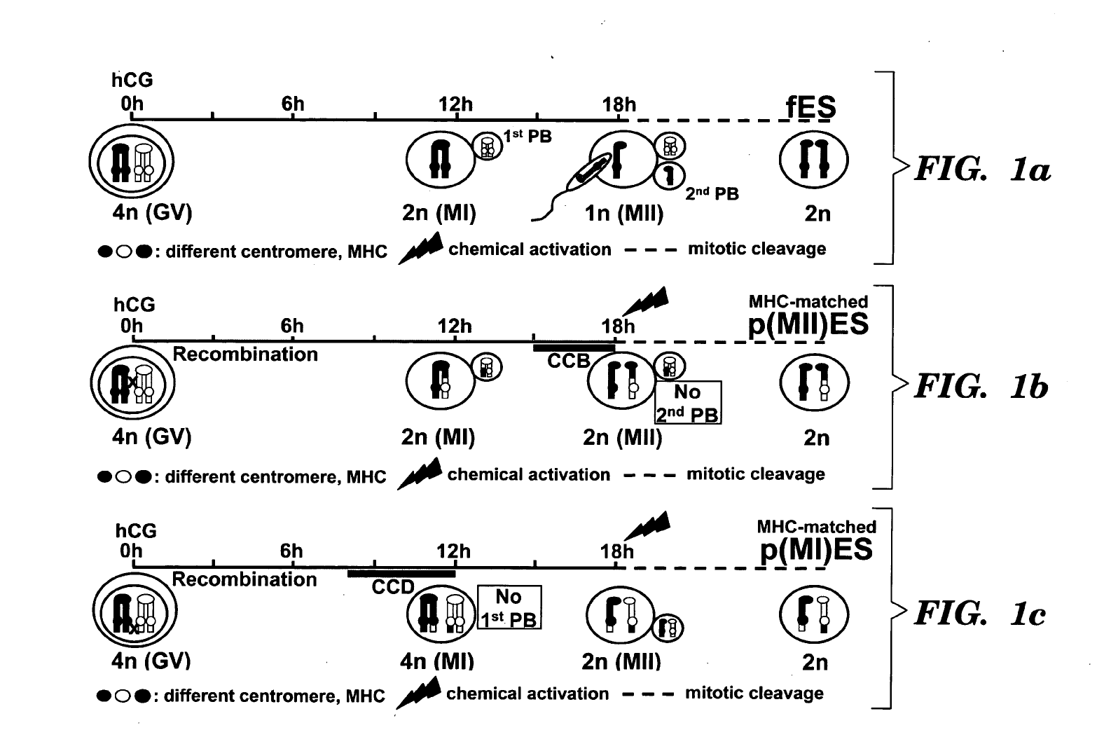

The isolated diploid oocyte that is in prophase or metaphase I of meiosis I is then cultured under conditions that inhibit formation of the first polar body. In this manner the first polar body, which in normal Meiosis contains a haploid DNA complement, is not extruded and the oocyte remains diploid (see FIGS. 1A and 1D).

In one embodiment, the formation of the first polar body is inhibited by incubation of cumulus-free oocytes with cytoclasin D (Sigma, C8273). For example, cytoclasin D can be added to suitable culture media at a concentration of 1-20 ug/ml for a sufficient period of time to inhibit formation. For example, in one embodiment oocytes are incubated with an inhibitor of first polar body formation for about 3 hours. In other embodiments cells are incubated with an inhibitor of first polar body formation for about 1 h, about 2 h, about 4 h, about 5 h, about 6 h, about 7 h, about 8 h, about 9 h, or about 10 hours.

In one embodiment, failed-to-fertilized oocytes are treated to induce parthenogenesis, e.g. human oocytes.

Isolated oocytes wherein the first polar body formation has been inhibited, are then activated to produce an embryo with a discernable trophectoderm and an inner cell mass. In one embodiment, activation is performed about 6 hours after inhibition of the first polar body formation. Activation can also be initiated at about 3 h, about 4 h, about 5 h, about 7 h, or about 8 h, after inhibition of first polar body formation. In one embodiment, activation is initiated at about 18 hours after induction of superovulation and injection with hCG. For example, if cumulus-free oocytes are isolated 9 hours after hCG injection and the cells are incubated with an inhibitor of first polar body formation for 4 h, then the cells are incubated in culture media for 5 hours before activation at an 18 h time point.

Means for activation of oocytes are known in the art and include, but are not limited to, mechanical methods such as pricking, manipulation or oocytes in culture, thermal methods such as cooling and heating, repeated electric pulses, enzymatic treatments such as trypsin, pronase, hyaluronidase, osmotic treatments, ionic treatments such as with divalent cations and calcium ionophores, the use of anaesthetics such as ether, ethanol, tetracaine, lignocaine, procaine, phenothiazine, tranquilizers such as thioridazine, trifluoperazine, fluphenazine, chlorpromazine, the use of protein synthesis inhibitors such as cycloheximide, puromycin, the use of phosphorylation inhibitors, e.g., protein kinase inhibitors such as DMAP, combinations thereof, as well as other methods. Such activation methods are well known in the art and are discussed U.S. Pat. No. 5,945,577 and U.S. Patent Application 2003/0129745, which are herein incorporated by reference.

In one embodiment, the oocytes are activated in suitable media containing 1-10 uM calcium ionophore A23187 for 0.5-5 minutes in air, then are incubated in 2 mM 6-dimethylaminopurine (6-DAMP) (SIGMA, D2629) dissolved in suitable media (e.g. KSOM) at 37° C. in 5% CO₂ for about 3 hours.

Other suitable activation procedures include, but are not limited to, activation by microinjection of adenophostin (a. Inject oocytes with 10 to 20 picoliters of a solution containing 10 uM of adenophostin, b. Place oocytes in culture); activation by microinjection of sperm factor (inject oocytes with 10 to 20 picoliters of sperm factor isolated either from primates, pigs, bovine, sheep, goat, horse, mice, rat, rabbit or hamster, b) place eggs in culture) or activation by microinjection of recombinant sperm factor.

Activation and parthogenetic procedures for mice, cows, monkeys have been described in Kaufman, M. H., et al., Establishment of pluripotential cell lines from haploid mouse embryos. J Embryol Exp Morphol, 1983. 73: p. 249-61; Wang, L., et al., Generation and characterization of pluripotent stem cells from cloned bovine embryos. Biol Reprod, 2005. 73(1): p. 149-55; Cibelli, J. B., et al., Parthenogenetic stem cells in nonhuman primates. Science, 2002. 295(5556): p. 819; and Vrana, K. E., et al., Nonhuman primate parthenogenetic stem cells. Proc Natl Acad Sci USA, 2003. 100 Suppl 1: p. 11911-6, which are herein incorporated by reference. A human parthogenic chimera has shown that human partogenetic embryonic stem cells can be used to produce blood stem cells, L., et al., A human parthenogenetic chimaera. Nat Genet, 1995. 11(2): p. 164-9. Parthenogenetic activation of human oocytes has also been described ³¹⁻⁴².

In one embodiment of the invention, an enucleated cell, e.g. mammalian oocyte, is transplanted with diploid DNA (e.g. two haploid DNA derived from oocytes (gynogenesis) or two sperm nuclei (androgenesis)), and is treated to prevent formation of the first polar body followed by an activation procedure to produce an embryo containing a discernible trophectoderm and inner cell mass which is incapable of giving rise to an offspring. Embryos generated in this manner using sperm DNA are referred to as androgenetic embryos. Embryos generated in this manner using two haploid female DNA are referred to as gynogenetic embryos. Gynogenetic embryos also refer to embryos obtained using a female oocyte isolated from an individual female containing original oocyte DNA.

The inner cell mass, or cells derived therefrom, are useful for obtaining pluripotent cells which may be maintained for prolonged periods in tissue culture.

In all cases, the activated oocyte which is diploid, is allowed to develop into an embryo that comprises a trophectoderm and an inner cell mass. This can be affected using known methods and culture media that facilitate blastocyst development. Examples thereof are disclosed in U.S. Pat. No. 5,945,577, and have been well reported in the literature. Culture media suitable for culturing and maturation of embryos are well known and include Ham's F-10+10% fetal calf serum, Tissue Culture Medium, 199 (TCM-199)+10% fetal calf serum, Tyrodes-Albumin-Lactate-Pyruvate (TALP), Dulbecco's Phosphate Buffered Saline (PBS), Eaglets and Whitten's media, and CR1 medium. A preferred medium is for bovine embryos TCM-199 with Earl salts, 10% fetal calf serum, 0.2 mM Na pyruvate and 50 .mu.g/ml gentamycin sulfates. A preferred medium for culturing pig embryos is NCSU23.

Preferred medium for culturing primate embryos, e.g., human and non-human primate embryos, include modified Ham's F-10 medium (Gibco, Catalog No. 430-1200 EB) supplemented with 1 ml/L synthetic serum replacement (SSR-2, Medl-Cult Denmark), and 10 mg/ml HSA; 80% Dulbecco's modified Eaglet's medium (DMEA, no pyruvate, high glucose formulation, Gibco BRL) with 20% fetal bovine serum, 0.1 mM B-mercaptoethanol, and 1% non-essential amino acid stock, and by methods and medium disclosed in Jones et al, Human Reprod. 13(1):169-177 (1998); Thomson et al, Proc. Natl. Acad. Sci., USA, 92:7894-7898 (1995); and Thomson et al, Science, 282:1145-1147 (1998); and two media available from Irvine Scientific, Santa Anna, Calif., i.e., a first media called P-1 (Cat. #99242) which is used for the first three days of culture followed by a second media, P-2 (Cat. #99292) until blastocyst stage.

After the androgenetic or gynogenetic embryos have been cultured to produce a discernable trophectoderm and inner cell mass, the cells of the inner cell mass are isolated and used to produce the desired pluripotent or totipotent cell lines, i.e. cells are cultured in media to maintain an undifferentiated state. This can be accomplished by transferring cells derived from the inner cell mass or the entire inner cell mass into a culture that inhibits differentiation. In one embodiment, this is effected by transferring said inner cell mass cells onto a feeder layer that inhibits differentiation, e.g., fibroblasts or epithelial cells, such as fibroblasts derived from murines, ungulates, chickens, such as mouse or rat fibroblasts, 570 and SI-m220 feeder cells, BRL cells, etc., or other cells that produce LIF. In one embodiment, the inner cell mass cells are cultured on mouse fetal fibroblast cells or other cells which produce leukemia inhibitory factor, or in the presence of leukemia inhibitory factor. Culturing will be effected under conditions that maintain said cells in an undifferentiated, pluripotent state, or totipotent state, for prolonged periods, theoretically indefinitely.

Suitable conditions for culturing pluripotent cells, specifically pluripotent cells derived from ungulate inner cell mass are also described in U.S. Pat. No. 5,945,577, as well as U.S. Pat. No. 5,905,042, both of which are incorporated by reference herein in their entirety.

In one embodiment, the DNA derived from an individual male or female is genetically modified before or after activation of the cell containing same, e.g., human oocyte. Methods and materials for effecting genetic modification are well known and include microinjection, the use of viral DNAs, homologous recombination, etc. Thereby, pluripotent or totipotent cells are obtained that comprise a desired DNA modification, e.g., contain a desired coding sequence.

It should be noted that, in embodiments of the invention, cells are obtained having DNA of either male or female origin which develop into an embryo having a discernible trophectoderm and inner cell mass which will not give rise to viable offspring. The inner cell mass or cells therein are used to produce pluripotent/totipotent cells containing cultures which are themselves useful for making differentiated cells and tissues.

In one embodiment, the substantially heterozygous pluripotent embryonic stem cells isolated by methods of the invention are further analyzed for heterozygosity at a desired locus. Methods for determining heterozygosity are well known to those in the art and include, but are not limited to genotype analysis such as polymerase chain reaction (PCR) amplification followed by allele-specific restriction enzyme digestion of a single nucleotide polymorphism (SNP) within the loci of interest; or PCR amplification combined with DNA chip analysis using specific oligonucleotides designed to detect unique sequences present in different loci alleles; or (PCR) amplification followed by restriction length polymorphism analysis; or Illumina multiplexed allele extension and ligation with detection using oligonucleotide probes covalently attached to beads which are assembled into fiber optic bundles (27, 28); or analysis using unique parental methylation marks and methylation sensitive restriction endonucleases; or detection of the allele specific protein expression and characteristics by electrophoresis, FACS analysis, immunostaining, and western blot.

In one embodiment, a heterozygous genotype is confirmed by monitoring gene expression. For example, to detect a heterozygous MHC phenotype, MHC antigen expression can be monitored in differentiated cells, e.g. using antibodies specific for particular MI-IC molecules.

The embryonic stem cell lines that are substantially heterozygous can be screened for heterozygosity at any desired gene loci. In one embodiment, the embryonic stem cell line that is substantially heterozygous is heterozygous for at least MI-IC loci (referred to herein as h-p(MI)ES cells). These cells are genetically matched to the donor DNA for at least one MHC loci. In one embodiment, when the donor DNA is human, the h-p(MI)ES cells are genetically matched for at least one HLA loci, e.g. at HLA-A, HLA-B, HLA-C, HLA-DR, HLA-DQ, or HLA-DP. In another embodiment, the pluripotent ES cells are genetically matched to the oocyte donor at each MHC loci (e.g. HLA loci) and have an identical MHC haplotype as the donor. Thus, the pluripotent ES cells provide a source for histocompatible tissues for transplantation.

In one embodiment, further genetic analysis of isolated of the p(MI)ES cells is performed to confirm a normal diploid content of the h-p(MI)ES cells. This can be done using means known in the art, e.g. direct chromosome counting or quantitative analysis of DNA content, e.g. using Hoechst 33342 stain.

In one embodiment, tetraploid cells that are heterogous at desired loci are selected.

In one embodiment, tetraploid cells that are heterozygous at MHC loci are selected (tetraploid h-p(MI)ES). As described in Example 1, histology analysis of teratomas revealed tissue elements of all three embryonic germ layers for each class of ES cell: mesoderm (bone, bone marrow, muscle and cartilage), endoderm (respiratory epithelium, exocrine pancreas) and ectoderm (brain, melanocyte (iris), and skin).

Method for Producing Stem Cells that are Heterozygous for at Least One MHC Locus by Screening for Cells where Donor MHC Loci have been Restored by Recombination

Another embodiment of the invention provides a method for producing stem cells that are heterozygous for at least one MHC locus. The method comprises a) obtaining oocyte cells in metaphase II that comprises haploid DNA derived from a single individual male or female, which optionally may be genetically modified; b) activating the oocyte cells of step (b) to induce parthenogenetic development under conditions that inhibit second polar body formation; c) culturing said activated oocytes to produce an embryos comprising a discernible, trophectoderm and an inner cell mass; d) isolating said inner cell mass, or cells therefrom, and transferring said inner cell mass, or cells, to an in vitro media that inhibits differentiation of said inner cell mass or cells derived therefrom thereby generating pluripotent embryonic stem (pES) cell lines; and e) selecting pES cell lines that have undergone recombination at least one MHC locus; and f) culturing the pES cells of step (e) to maintain said cells in an undifferentiated state thereby generating a pES cell line that is heterozygous for at least one MHC locus.

As used herein “heterozygous for at least one MHC locus” refers to a pES cell that has genetic identity to the donor DNA at least on MHC locus. In the parthenogenetic methods of the invention such genetic identity occurs through a recombination event occurring in meiosis I prior to replication of the haploid genotype.

Means for obtaining oocyte cells in metaphase II with haploid DNA content are known to those skilled in the art. In one embodiment, superovulation is induced with PMSG and the donor subject is injected with hCG 48 hours later. Oocytes are then collected 14-15 hours after hCG injection. The oocytes collected 14-15 hours after hCG injection will primarily be oocytes with haploid DNA content, i.e. the first polar body has extruded.

In one embodiment, e.g. for a human donor, PMSG is given on the second or third cycle day and given for 6-9 consecutive days followed by hCG injection. Oocytes are then collected 36-48 hours after hCG injection.

The haploid oocyte cells are then activated to undergo parthenogenetic development under conditions that inhibit second polar body formation. Means for parthenogenetic activation are well known to those skilled in the art, some of which are described in this application under the heading of “Methods for producing an embryonic cell line that is substantially heterozygous.”

In methods of the invention, the haploid oocyte cells are activated under conditions that inhibit formation of the second polar body. Classical parthenogenesis methods involve activation of oocytes under conditions that inhibit second polar body formation and are well known to those in the art. For example, this can be affected by various means including, but not limited to, the use of phosphorylation inhibitors such as DMAP or by use of a microfilament inhibitor such as cytochlasin B, C, or D, or a combination thereof.

In one embodiment, the haploid oocyte cells are activated in suitable media containing 10 uM calcium ionophore A23187 for 5 minutes in air, then incubated in 2 mM 6-dimethylaminopurine (6-DMAP) and 5 ug/ml of cytoclasin B for 3 hours to inhibit second polar body formation.

Cells are cultured under suitable conditions (see conditions described under the heading of “Methods for producing an embryonic cell line that is substantially heterozygous”) to allow development of an embryo that comprises a trophectoderm and an inner cell mass which is incapable of giving rise to an offspring. Cells are then isolated cells from the inner cell mass and cultured in media to maintain an undifferentiated state (e.g. see conditions described under the heading of “Methods for producing an embryonic cell line that is substantially heterozygous”).

In one embodiment the cells are cultures in the presence of MEF in serum free ES maintenance media (Gibco, 10829-018), 5% CO₂, O₂ and 90% N₂.

The pES cells are then screened for to select for pES cell lines that have undergone recombination at least one MHC locus resulting in a heterozygous MHC loci.

Methods for determining whether or not recombination has occurred at MHC loci, include but are not limited to MHC genotype analysis such as polymerase chain reaction (PCR) amplification followed by allele-specific restriction enzyme digestion of a single nucleotide polymorphism (SNP) within the MHC loci of interest; or PCR amplification combined with DNA chip analysis using specific oligonucleotides designed to detect unique sequences present in different MHC loci alleles; or (PCR) amplification followed by restriction length polymorphism analysis; or Illumina multiplexed allele extension and ligation with detection using specific MHC allele oligonucleotide probes covalently attached to beads which are assembled into fiber optic bundles (27, 28); or analysis using unique parental methylation marks and methylation sensitive restriction endonucleases. Means for determining heterozygosity at MHC loci are also described in Example I; or detection of the allele specific protein expression and characteristics by electrophoresis, FACS analysis, immunostaining, and western blot.

The MHC genes are polygenic—each individual possesses multiple, different MI-IC class I and MHC class II genes. The MHC genes are also polymorphic—many variants of each gene are present in the human and non-human population. In fact, the MHC genes are the most polymorphic genes known. Each MHC Class I receptor consists of a variable a chain and a relatively conserved β-2-microglobulin chain. Three different, highly polymorphic class I α chain genes have been identified. These are called HLA-A, HLA-B, and HLA-C. Variations in the α chain chains account for all of the different class I MHC genes in the population. MHC Class II receptors are also made up of two polypeptide chains, an α chain and a β chain, both of which are polymorphic. In humans, there are three pairs of MHC class II α and β chain genes, called HLA-DR, HLA-DP, and HLA-DQ. Frequently, the HLA-DR cluster contains an extra gene encoding a β chain that can combine with the DR α chain; thus, an individual's three MHC Class II genes can give rise to four different MHC Class II molecules.

Human MHC loci are clustured on the short arm of chromosome 6 in a region that extends over from 4 to 7 million base pairs that is called the major histocompatibility complex. Because there so many different variants of MHC alleles in the human population, most people have heterozygous MHC alleles.

HLA-A belongs to the HLA class I heavy chain paralogues; GeneID: 3105, Locus tag: DAQB-90C11.16, Genbank reference sequence NG_{—}002398 Hundreds of HLA-A alleales have been described. Typing for these polymorphisms is routinely done by those skilled in the art for bone marrow and kidney transplantation.

HLA-B belongs to the HLA class I heavy chain paralogues; GeneID: 3106 Locus tag: DAQB-48K1, Genbank reference sequence NG_{—}002397. Hundreds of HLA-B alleales have been described. Typing for these polymorphisms is routinely done by those skilled in the art for bone marrow and kidney transplantation.

HLA-C belongs to the HLA class I heavy chain paralogues; GeneID: 3107, Genbank reference sequence NG_{—}002397. Over one hundred HLA-C alleles have been described. Typing for these allelic polymorphisms is routinely done by those skilled in the art for bone marrow and kidney transplantation.

HLA-DRB1 belongs to the HLA class II beta chain paralogues; GeneID: 3123, Locus tag: XXbac-BPG161M6.1, Genbank reference sequence NG_{—}002432. Hundreds of DRB1 alleles have been described and typing for these polymorphisms is routinely done for bone marrow and kidney transplantation.

HLA-DQB1 belongs to the HLA class II beta chain paralogues; GeneID: 3119, Locus tag: DAQB-109B10.2, Genbank reference sequence NM_{—}002123. Within the DQ molecule both the alpha chain and the beta chain contain the polymorphisms specifying the peptide binding specificities, resulting in up to 4 different molecules. Typing for these polymorphisms is routinely done for bone marrow transplantation.

HLA-DQA1 belongs to the HLA class II alpha chain paralogues; GeneID: 3117, Genbank reference sequence NM_{—}002122. The class II molecule is a heterodimer consisting of an alpha (DQA) and a beta chain (DQB), both anchored in the membrane. Within the DQ molecule both the alpha chain and the beta chain contain the polymorphisms specifying the peptide binding specificities, resulting in up to four different molecules. Typing for these polymorphisms is routinely done for bone marrow transplantation.

HLA-DPB1 belongs to the HLA class II beta chain paralogues; GeneID: 3115 Locus tag: DAQB-79P13.4, Genbank reference sequence NM_{—}002121 This class II molecule is a heterodimer consisting of an alpha (DPA) and a beta chain (DPB), both anchored in the membrane. Within the DP molecule both the alpha chain and the beta chain contain the polymorphisms specifying the peptide binding specificities, resulting in up to 4 different molecules.

HLA-DPA1 belongs to the HLA class II alpha chain paralogues; GeneID: 3113, Genbank reference sequence NM_{—}033554. Within the DP molecule both the alpha chain and the beta chain contain the polymorphisms specifying the peptide binding specificities, resulting in up to 4 different molecules.

A database of HLA Class I and Class II alleles is maintained by the informatics group of Anthony Nolan Trust, the Royal Free Hospital, Hampstead, London, England. As of July 2006, the data base contains 2,532 HLA allele sequences (http://www.ebi.ac.uk/imgt/hla/intro.html).

In one embodiment, a heterozygous MHC genotype is confirmed by monitoring MHC antigen expression in differentiated cells, e.g. using antibodies specific for particular MHC molecules.

After cells, which are heterozygous for at least one MHC loci, are selected that pES cells are cultured to maintain their undifferentiated state thereby generating a pES cell line that is heterozygous for at least one MI-IC locus. Means for maintaining cells in undifferentiated states are well known in the art, some of which are described below under the heading “Preparing Totipotent and/or Pluripotent Stem Cells.”

In one embodiment, maintaining an undifferentiated state is effected by transferring the cells onto a feeder layer that inhibits differentiation, e.g., fibroblasts or epithelial cells, such as fibroblasts derived from murines, ungulates, chickens, such as mouse or rat fibroblasts, 570 and SI-m220 feeder cells, BRL cells, etc., or other cells that produce leukemia inhibitory factor (LIF). In one embodiment, the pES is cultured in the presence of MEF in serum free ES maintenance media (GIBCO, 10829-018) in 5% CO₂, O₂ and 90% N₂. In one embodiment, the cells are cultured on mouse fetal fibroblast cells or other cells which produce LIF, or are cultured in the presence of leukemia inhibitory factor. Culturing will be effected under conditions that maintain said cells in an undifferentiated, pluripotent state, or totipotent state, for prolonged periods, theoretically indefinitely.

Pluripotent ES cells that are heterozygous at least one MHC loci are referred to herein as h-p(MII) ES cells.

In one embodiment, further genetic analysis of isolated is performed to confirm a normal diploid content of the h-p(MII)ES cells.

In one embodiment, tetraploid cells that are heterogous for at least one MI-IC loci are selected (tetraploid h-p(MII)ES). As described in Example 1, histology analysis of teratomas revealed tissue elements of all three embryonic germ layers for each class of ES cell: mesoderm (bone, bone marrow, muscle and cartilage), endoderm (respiratory epithelium, exocrine pancreas) and ectoderm (brain, melanocyte (iris), and skin).

Peri-Centromeric Genotype Analysis

In another embodiment of the invention, a method is provided for determining if a pES cell line is derived from a parthenogenesis embryo, nuclear transfer embryo, a natural fertilization embryo.

SNP genotyping is performed at various distances along the chromosome using methods known in the art, for example as described in example 1. Plotting the heterozygous rate (heterozygous SNP markers/total SNP makers) versus SNP marker distance from centromere on a graph (X axis is the heterozygous rate and the Y axis is the SNP marker distance from centromereresults) reveals distinct patterns of homozygosity and heterozygosity.

As used herein, “parthenogenesis embryo” refers to an embryo that is produced by parthenogenetic activation of an oocyte; including parthogenesis when first polor body formation is inhibited (from which (pMI)ES cells are derived) and parthogenesis when second polor body formation is inhibited (from which (pMII)ES cells are derived).

As used herein, “nuclear transfer embryo” refers to an embryo that is produced by the fusion or transplantation of a donor cell or DNA from a donor cell into a suitable recipient cell, typically an oocyte of the same or different species that is treated before, concomitant or after transplant or fusion to remove or inactivate its endogenous nuclear DNA. The donor cell used for nuclear transfer include embryonic and differentiated cells, e.g., somatic and germ cells. The donor cell may be in a proliferative cell cycle (G1, G2, S or M) or non-proliferating (Go or quiescent).

As used herein, “natural fertilization embryo” refers to an embryo that is produced by natural fertilization of an oocyte, i.e. sperm fertilization.

If homozygosity predominates near the centromere and heterozygosity is observed with increasing frequency at telomeric loci, then a p(MII)ES cell line has been derived by parthenogenetic activation of the oocyte following completion of the first meiotic segregation of homologous chromosomes. However, if heterozygosity predominates near the centromere with increasing frequency of homozygosity at markers distal to the centromere, then a p(MI)ES cell line has resulted from a disruption in segregation of the homologous chromosomes that normally occurs in MI, followed by centromere separation and sister chromatid segregation into diploid progeny during the artificial activation and oocyte maturation process. A cell line derived from an embryo produced by nuclear transfer from a somatic cell will be, for the most part, a complete genetic match of the nuclear donor, as only rare occurrences of mitotic recombination would alter the expected pattern of heterozygosity. Furthermore, there will be no discernable pattern of heterozygosity relative to centromeric distance. Similarly an ES cell line derived from a fertilized blastocyst will be a combination of sperm and egg donor haplotypes, again with no relationship between frequency of heterozygosity of markers and distance from the centromere.

Thus, in p(MII)ES cells the frequency of heterozygous SNPs increases in proportion to the distance from the centromere, resulting in a positive slope when heterozygosity (y axis) versus marker distance from centromere (x axis) is plotted. In p(MI)ES cells the frequency of homozygous SNPs increases in proportion to the distance from the centromere, resulting in a negative slope when heterozygosity (y axis) versus marker distance from centromere (x axis) is plotted. In pES cells derived from natural fertilization the graph will show no relationship between frequency of heterozygosity of markers and distance from the centromere.

Preparing Totipotent and/or Pluripotent Stein Cells

Stem cells are present in the earliest stages of embryo formation. Embryonic stem cells (ES cells) are undifferentiated stem cells that are derived from the inner cell mass (ICM) of a blastocyst embryo. Totipotent and/or nearly totipotent ES cell lines can be derived from human blastocysts using known methods comprising removing cells of the inner cell mass of an early blastocyst by microsurgery or immunosurgery and culturing the cells in vitro (e.g., see U.S. Pat. No. 6,235,970, the contents of which are incorporated herein by reference in their entirety). For example, such methods are described in PCT application, PCT/USO2/37899 (Methods for Making and Using Reprogrammed Human Somatic Cell Nuclei and Autologous and Isogenic Stem Cells) filed Nov. 26, 2002, using blastocysts produced both by parthenogenesis, the disclosure of which are incorporated herein by reference in its entirety. Thomson et al. also describes methods by which ES cell lines can be derived from primate/human blastocysts (Science, 1988, 282:1145-1147; and Proc. Natl. Acad. Sci., USA, 1995, 92:7544-7848), which are incorporated by reference herein in their entirety. A detailed method for preparing human ES cells is also described in Thomson's U.S. Pat. No. 6,200,806, “Primate Embryonic Cells,” issued Mar. 13, 2001, the disclosure of which is incorporated herein by reference in its entirety.

In one embodiment, a human ES cell line is derived from cells of a blastocyst by a method comprising: a. isolating a human blastocyst; b. isolating cells from the inner cell mass of the blastocyst; c. plating the inner cell mass cells on embryonic fibroblasts so that inner-cell mass-derived cell masses are formed; d. dissociating the mass into dissociated cells; e. replating the dissociated cells on embryonic feeder cells; f. selecting colonies with compact morphologies and cells with high nucleus to cytoplasm ratios and prominent nucleoli; and g. culturing the selected cells to generate a pluripotent human embryonic stem cell line.

Methods for growing human ES cells and maintaining them in an undifferentiated state without culturing them on a layer of feeder cells have also been described (Xu et al., Nature Biotechnology, 2001, 19:971-4, the contents of which are incorporated herein by reference in their entirety). Feeder-free culture of stem cells can reduce the risk of contamination of the cells by pathogens that may reside in the feeder cells.

Generating Differentiated Cells

Stem cells are widely regarded as an abundant source of pluripotent cellular material that can be directed to differentiate into cells and tissues that are suitable for transplantation into patients in need of such cell and tissue transplants. ES cells appear to have unlimited proliferative potential and are capable of differentiating into all of the specialized cell types of a mammal, including the three embryonic germ layers (endoderm, mesoderm, and ectoderm), and all somatic cell lineages and the germ line. Using known methods, totipotent or nearly totipotent ES cells can be cultured under conditions in which they differentiate into pluripotent or multipotent stem cells such as hematopoietic or neuronal stem cells. Alternatively, totipotent ES cells can be cultured under conditions in which they differentiate into a terminally differentiated cell type such as a cardiac muscle cell.

Totipotent and/or pluripotent stem cells with a substantially heterozygous genome (e.g. at the human leukocyte antigen (HLA) loci) produced by methods of the invention can be cultured using methods and conditions known in the art to generate cell lineages that differentiate into many, if not all, of the cell types of the body, for transplant into human patients in need of such transplants. Such stem cells having substantially heterozygous genome (e.g. at the human leukocyte antigen (HLA) loci) can differentiate into cells selected from the group consisting of immune cells, neurons, skeletal myoblasts, smooth muscle cells, cardiac muscle cells, skin cells, pancreatic islet cells, hematopoietic cells, kidney cells, and hepatocytes. For example, methods have been described by which totipotent or nearly totipotent ES cells are induced to differentiate in vitro into cardiomyocytes (Paquin et al., Proc. Nat. Acad. Sci. (2002) 99:9550-9555), hematopoietic cells (Weiss et al., Hematol. Oncol. Clin. N. Amer. (1997) 11(6):1185-98; also U.S. Pat. No. 6,280,718), insulin-secreting beta cells (Assady et al., Diabetes (2001) 50(8):1691-1697), and neural progenitors capable of differentiating into astrocytes, oligodendrocytes, and mature neurons (Reubinoff et al., Nature Biotechnology (2001) 19:1134-1140; also U.S. Pat. No. 5,851,832).

The pluripotent state of the cells produced by embodiments of the invention can be confirmed by various methods.

For example, the cells can be tested for the presence or absence of characteristic ES cell markers. In the case of human ES cells, examples of such markers are identified supra, and include SSEA-4, SSEA-3, TRA-1-60 and TRA-1-81 and are known in the art.

Also, pluripotency can be confirmed by injecting the cells into a suitable animal, e.g., a SCID mouse, and observing the production of differentiated cells and tissues. Still another method of confirming pluripotency is using the subject pluripotent cells to generate chimeric animals and observing the contribution of the introduced cells to different cell types. Methods for producing chimeric animals are well known in the art and are described in our related applications, incorporated by reference herein.

Yet another method of culturing pluripotency is to observe their differentiation into embryoid bodies and other differentiated cell types when cultured under conditions that favor differentiation (e.g., removal of fibroblast feeder layers). This method has been utilized in the present invention and it has been confirmed that the subject pluripotent cells give rise to embryoid bodies and different differentiated cell types in tissue culture. For example, it has been shown that Cynomolgous pluripotent cell lines produced herein give rise to beating cardiomyctes and other cells when allowed to differentiate by culturing of the cell line beyond confluency.

The resultant pluripotent cells and cell lines, preferably human pluripotent cells and cell lines, which are substantially heterozygous, particularly cells heterozygous at MHC Loci have numerous therapeutic and diagnostic applications. Most especially, such pluripotent cells may be used for cell transplantation therapies or gene therapy (if genetically modified). Human ES cells have application in the treatment of numerous disease conditions.

In this regard, it is known that mouse embryonic stem (ES) cells are capable of differentiating into almost any cell type, e.g., hematopoietic stem cells. Therefore, human or other mammalian pluripotent (ES) cells produced according to methods of the invention should possess similar differentiation capacity. The pluripotent cells according to the invention will be induced to differentiate to obtain the desired cell types according to known methods. For example, human ES cells produced according to methods of the invention may be induced to differentiate into hematopoietic stem cells, muscle cells, cardiac muscle cells, liver cells, cartilage cells, epithelial cells, urinary tract cells, etc., by culturing such cells in differentiation medium and under conditions which provide for cell differentiation. Medium and methods which result in the differentiation of ES cells are known in the art, as are suitable culturing conditions.

For example, Palacios et al, Proc. Natl. Acad. Sci., USA, 92:7530-7537 (1995) teaches the production of hematopoietic stem cells from an embryonic cell line by subjecting stem cells to an induction procedure comprising initially culturing aggregates of such cells in a suspension culture medium lacking retinoic acid followed by culturing in the same medium containing retinoic acid, followed by transferral of cell aggregates to a substrate which provides for cell attachment.

Moreover, Pedersen, J. Reprod. Fertil. Dev., 6:543-552 (1994) is a review article which references numerous articles disclosing methods for in vitro differentiation of embryonic stem cells to produce various differentiated cell types including hematopoietic cells, muscle, cardiac muscle, nerve cells, among others.

Further, Bain et al, Dev. Biol, 168:342-357 (1995) teaches in vitro differentiation of embryonic stem cells to produce neural cells which possess neuronal properties. These references are exemplary of reported methods for obtaining differentiated cells from embryonic or stem cells. These references and in particular the disclosures therein relating to methods for differentiating embryonic stem cells are incorporated by reference in their entirety herein.

Thus, using known methods and culture medium, one skilled in the art may culture the subject ES cells, including genetically engineered or transgenic ES cells, to obtain desired differentiated cell types, e.g., neural cells, muscle cells, hematopoietic cells, etc.

Pluripotent cells produced by the invention may be used to obtain any desired differentiated cell type. Therapeutic usages of differentiated human cells are unparalleled. For example, human hematopoietic stem cells may be used in medical treatments requiring bone marrow transplantation. Such procedures are used to treat many diseases, e.g., late stage cancers such as ovarian cancer and leukemia, as well as diseases that compromise the immune system, such as AIDS. Hematopoietic stem cells can be obtained, e.g., by incorporating male or female DNA derived from a male or female cancer or AIDS patient with an enucleated oocyte, obtaining pluripotent cells as described above, and culturing such cells under conditions which favor differentiation, until hematopoietic stem cells are obtained. Such hematopoietic cells may be used in the treatment of diseases including cancer and AIDS.

Alternatively, the subject pluripotent cells may be used to treat a patient with a neurological disorder by culturing such cells under differentiation conditions that produce neural cell lines. Specific diseases treatable by transplantation of such human neural cells include, by way of example, Parkinson's disease, Alzheimer's disease, ALS and cerebral palsy, among others. In the specific case of Parkinson's disease, it has been demonstrated that transplanted fetal brain neural cells make the proper connections with surrounding cells and produce dopamine. This can result in long-term reversal of Parkinson's disease symptoms.

In one embodiment, the pluripotent ES cells derived by the methods described herein are used to create a Stem cell bank containing a library or plurality of human or non-human animal embryonic stem cell lines.

In embodiments of the invention are provided methods to generate pluripotent ES cells that are genetically matched to the oocyte donor at the MHC loci. These methods provide an essentially limitless supply of pluripotent stem cells, e.g. pluripotent human cells that can be used to produce differentiated cells suitable for transplantation. Such cells should alleviate the significant problem associated with current transplantation methods, i.e., rejection of the transplanted tissue which may occur because of host-vs.-graft or graft-vs.-host rejection. Conventionally, rejection is prevented or reduced by the administration of anti-rejection drugs such as cyclosporin. However, such drugs have significant adverse side-effects, e.g., immunosuppression, carcinogenic properties, as well as being very expensive. The MHC donor matched cells of the invention should eliminate, or at least greatly reduce, the need for anti-rejection drugs.

Other diseases and conditions treatable by cell therapy include, by way of example, spinal cord injuries, multiple sclerosis, muscular dystrophy, diabetes, liver diseases, i.e., hypercholesterolemia, diabetes, heart diseases, cartilage replacement, burns, foot ulcers, gastrointestinal diseases, vascular diseases, kidney disease, urinary tract disease, and aging related diseases and conditions.

This methodology can be used to replace defective genes, e.g., defective immune system genes, cystic fibrosis genes, or to introduce genes which result in the expression of therapeutically beneficial proteins such as growth factors, lymphokines, cytokines, enzymes, etc. For example, the gene encoding brain derived growth factor may be introduced into human pluripotent cells produced according to the invention, the cells differentiated into neural cells and the cells transplanted into a Parkinson's patient to retard the loss of neural cells during such disease.

Previously, cell types transfected with BDNF varied from primary cells to immortalized cell lines, either neural or non-neural (myoblast and fibroblast) derived cells. For example, astrocytes have been transfected with BDNF gene using retroviral vectors, and the cells grafted into a rat model of Parkinson's disease (Yoshimoto et al., Brain Research, 691:25-36, (1995)).

This ex vivo therapy reduced Parkinson's-like symptoms in the rats up to 45% 32 days after transfer. Also, the tyrosine hydroxylase gene has been placed into astrocytes with similar results (Lundberg et al., Develop. Neurol., 139:39-53 (1996) and references cited therein).

However, such ex vivo systems have problems. In particular, retroviral vectors currently used are down-regulated in vivo and the transgene is only transiently expressed (review by Mulligan, Science, 260:926-932 (1993)). Also, such studies used primary cells, astrocytes, which have finite life span and replicate slowly. Such properties adversely affect the rate of transfection and impede selection of stably transfected cells. Moreover, it is almost impossible to propagate a large population of gene targeted primary cells to be used in homologous recombination techniques. By contrast, the difficulties associated with retroviral systems should be eliminated by the use of the methods herein.

Genes which may be introduced into the subject pluripotent cells include, by way of example, epidermal growth factor, basic fibroblast growth factor, glial derived neurotrophic growth factor, insulin-like growth factor (I and II), neurotrophin-3, neurotrophin-4/5, ciliary neurotrophic factor, AFT-1, cytokine genes (interleukins, interferons, colony stimulating factors, tumor necrosis factors (alpha and beta), etc.), genes encoding therapeutic enzymes, etc.

In addition to the use of human pluripotent cells and cells derived therefrom in cell, tissue and organ transplantation, the present invention also includes the use of non-human cells in the treatment of human diseases. For example, non-human primate pluripotent cells produced according to the invention should be useful for treatment of human disease conditions where cell, tissue or organ transplantation is warranted (given the phylogenetic closeness of primates and humans (immunogenicity should be less of a concern.) In general, pluripotent cells and differentiated cells derived therefrom produced according to the present invention can be used within the same species (autologous, syngenic or allografts) or across species (xenografts). For example, brain cells derived from bovine or porcine pluripotent cells may be used to treat Parkinson's disease.

Also, the subject pluripotent ES cells, preferably human cells, may be used as an in vitro model of differentiation, in particular for the study of genes which are involved in the regulation of early development. The pluripotent ES cells, can further be used as an in vitro model for different diseases, in particular for the study of genes and processes contributing to the pathogenesis of the disease (e.g. neurodegenerative diseases, (Parkinson's, Alzheimer's, ALS etc.) and diabetes etc.). Also, differentiated cell tissues and organs produced using the subject ES cells may be used in drug studies.

Further, the subject ES cells or differentiated cells derived therefrom may be used as nuclear donors for the production of other ES cells and cell colonies, or in the case of non-human cells, for the production of cloned animals.

Still further, pluripotent cells obtained according to the invention may be used to identify proteins and genes that are involved in embryogenesis. This can be effected e.g. by differential expression, i.e. by comparing mRNA's that are expressed in pluripotent cells provided according to the invention to mRNAs that are expressed as these cells differentiate in to different cell types, e.g., neural cells, myocardiocytes, other muscle cells, skin cells, etc. Thereby, it may be possible to determine what genes are involved in differentiation of specific cell types.

Also, it is another object of the invention to expose pluripotent cell lines produced according to the invention to cocktails of different growth factors, at different concentrations so as to identify conditions that induce the production and proliferation of desired differentiated cell types.

In one embodiment of the invention, a stem cell bank is produced that comprises hematopoietic stem cells heterozygous for MHC antigens. A method for inducing the differentiation of pluripotent human embryonic stem cells into hematopoietic cells useful for transplant according to the present invention is described in U.S. Pat. No. 6,280,718, “Hematopoietic Differentiation of Human Pluripotent Embryonic Stem Cells,” issued to Kaufman et al. on Aug. 28, 2001, the disclosure of which is incorporated herein by reference in its entirety. The method disclosed in the patent of Kaufman et al. comprises exposing a culture of pluripotent human embryonic stem cells to mammalian hematopoietic stromal cells to induce differentiation of at least some of the stem cells to form hematopoietic cells that form hematopoietic cell colony forming units when placed in methylcellulose culture.

### Example 1

### Heterozygous Parthogenic Embryonic Stem Cells

### Methods

SNP Detection by Restriction Enzyme Digestion of PCR Amplicons.

The variants of the H2K gene (MHC class I antigens) were amplified by PCR. Exon-spanning oligonucleotides were designed in order to flank restriction site variants for BsiE1 (specific for H-2 Kb). The sense oligonucleotide (CCTGGGCTTCTACCCTGCT) (SEQ ID NO: 66) is located in exon 4, the anti-sense primer (CCACCACAGCTCCAGTGAC) (SEQ ID NO: 67) in exon 5 of the H-2K gene. PCR was carried out with 50 ng genomic DNA. PCR reactions were set up in a total volume of 50 ml reaction mix containing 2 units of AmpliTaq DNA polymerase (Applied Biosystems [Perkin Elmer], Weiterstadt, Germany). PCR cycling was performed using the following protocol: 94° C. for 4 min (initial denaturation); 92 C for 40° sec, annealing 60° C. for 40 sec, 72° C. for 40 sec (35 cycles); 72° C. for 10 min (final elongation). PCR products were purified using Qiaquick PCR purification kit (Qiagen, Valencia, Calif., USA). Purified PCR products were digested with BsiE1 (NEB, Beverly, Mass., USA) for 8 hours, and loaded on an agarose gel (Cambrex BioScience Rockland, 50180). Lane 1: 100 bp size marker, lane 2: uncut PCR product, lane 3: uncut spiked DNA, lane 4: C57BL/6 BsiEI digested, lane 5: CBA BsiEI digested, lane 5-13: nine p(MII)ES cells from B6CBAF1 mice BsiEI digested, lane 14-18: five p(MI)ES cells from B6CBAF1 mice BsiEI digested. As internal controls for the completion of restriction enzyme digestions, we spiked in a DNA fragment (arrow) containing BsiEI restriction enzyme sites, to indicate complete digestion. Spiked DNA was made by PCR amplification of puc19 plasmid using the primer set, CCTCCGATCGTTGTCAGAAG (SEQ ID NO: 68) and CTGGCGTAATAGCGAAGAG (SEQ ID NO: 69).

The variants of the Tap1 gene were amplified by PCR using the primer set, AAGAGCACCGTGGCTGCC (SEQ ID NO: 70) and GTGCAGGTAATGATGATCATA (SEQ ID NO: 71). PCR was carried out with 50 ng genomic DNA. PCR reactions were set up in a total volume of 50 ml reaction mix containing 2 units of AmpliTaq DNA polymerase (Applied Biosystems [Perkin Elmer], Weiterstadt, Germany). PCR cycling was performed using the following protocol: 94° C. for 4 min (initial denaturation); 92 C for 30 sec, annealing 55° C. for 30 sec, 72° C. for 60° sec (35 cycles); 72° C. for 10 min (final elongation). PCR products were purified using Qiaquick PCR purification kit (Qiagen, Valencia, Calif., USA). Purified PCR products were digested with Hha1 (NEB, Beverly, Mass., USA) for 8 hours, and loaded into agarose gel (Cambrex BioScience Rockland, 50180). Lane 1: 100 bp size marker, lane 2: uncut PCR product, lane 3: uncut spiked DNA, lane 4: spiked DNA HhaI digested, lane 5: C57BL/6 HhaI digested, lane 6: CBA HhaI digested, lane 7: B6CBAF1 HhaI digested, lane 8-13: six h-p(MI)ES cells from B6CBAF1 mice HhaI digested, lane 14: 100 bp size marker. As internal controls for the completion of restriction enzyme digestions, we spiked in a DNA fragment containing HhaI restriction enzyme sites, to indicate complete digestion.

p(MII)ES Cell Derivation.

Hybrid B6CBAF1 mice (C57BL/6×CBA) (Jackson Laboratories) were used as oocyte donors. Eight to ten week old female mice were superovulated by injection of 5 IU Pregnant mare serum gonadotropin (PMSG, Calbiochem 367222) and 48 h later, 5 IU Human chorionic gonadotropin (hCG, Calbiochem 230734). Oocytes were collected 14-15 hours after hCG injection. Oocytes with cumulus cells were activated in KSOM (Specialty Media, MR-106-D) containing 10 mM calcium ionophore A23187 (Sigma, C7522) for 5 min in air, then in 2 mM 6-dimethylaminopurine (6-DMAP) (Sigma, D2629) and 5 mg/ml of cytochalasin B (Sigma, C6762) dissolved in KSOM at 37° C. in 5% CO2 for 3 hours. Embryos were then washed five times in 500 micro liters of KSOM. Embryos were cultured in KSOM. All cultures were performed at 37° C. in 5% CO2, 5% O2, and 90% N2. Two and four days after activation stages and rate of embryo development evaluated under a stereomicroscope. The zona pellucida of the blastocysts were removed in 1% pronase in FHM mdia (Specialty Media, MR-024-D), and cells were cultured in the presence of MEF in serum free ES maintenance media (Gibco, 10829-018) in 5% CO2, 5% O2, and 90% N2.

p(MI)ES Cell Derivation.

Eight to ten week old female mice were superovulated by injection of 5 IU PMSG, and 48 h later, 5 IU hCG. Oocytes were collected from ovary within 9 hours after hCG injection. Cumulus cells were dispersed by incubation in hyaluronidase (Sigma, H4272: 1 mg/ml in KSOM) for 2-5 minutes at 37° C. for 5 min. Cumulus-free oocytes were then washed five times in 500 micro liters of KSOM. The cumulus cell free oocytes were incubated in KSOM containing 5 mg/ml of cytochalasin D (Sigma, C8273) for 3 hours. Cumulus-free oocytes were then washed five times in 500 micro liters of KSOM and incubated in KSOM at 37° C. in 5% CO2 for 6 hours. The oocytes were activated in KSOM containing 10 mM calcium ionophore A23187 for 5 min in air, then in 2 mM 6-dimethylaminopurine (6-DMAP) (Sigma, D2629) dissolved in KSOM at 37° C. in 5% CO2 for 3 hours. Embryos were then washed five times in 500 micro liters of KSOM. All cultures were performed in culture condition at 37° C. in 5% CO2, 5% O2, and 90% N2 in serum free ES maintenance media, which enhanced ES cell isolation efficiency.

Histopathology of Teratomas Made with h-p(MI)ES Cells, and Skin Chimerism.

10⁶ ES cells were injected subcutaneously into immunodeficient Rag2^{-/-} γC^{-/-} mice. 4 weeks after injection, teratomas were excised and fixed in 4% formaldehyde solution (Sigma, HT50-1-2). Pathological analysis was performed on 15 teratomas from p(MII)ES/h-p(MII)ES/h-p(MI)ES/fES/tetraploid ES cells by the Dana-Farber/Harvard Cancer Center Research Pathology Cores. Comparable teratomas were observed in all cases. a, cartilage. b, bone and bone marrow. c, muscle (1). d, brain. e, melanocyte (iris). f, skin. g, respiratory epithelium. h, pancreas. i, p(I)ES cells were injected into recipient blastocysts from the BalbCSJLF1 mouse (white coat color) to monitor the skin chimerism. A high degree of skin chimerism was observed, but no germ line transmission was demonstrated in over 700 progeny. No full-term mouse pups were obtained after injection of h-p(MI)ES cells into 50 tetraploid embryos (30).

### Results

Isolation of Histocompatible (h-) p(MII)ES Cells.

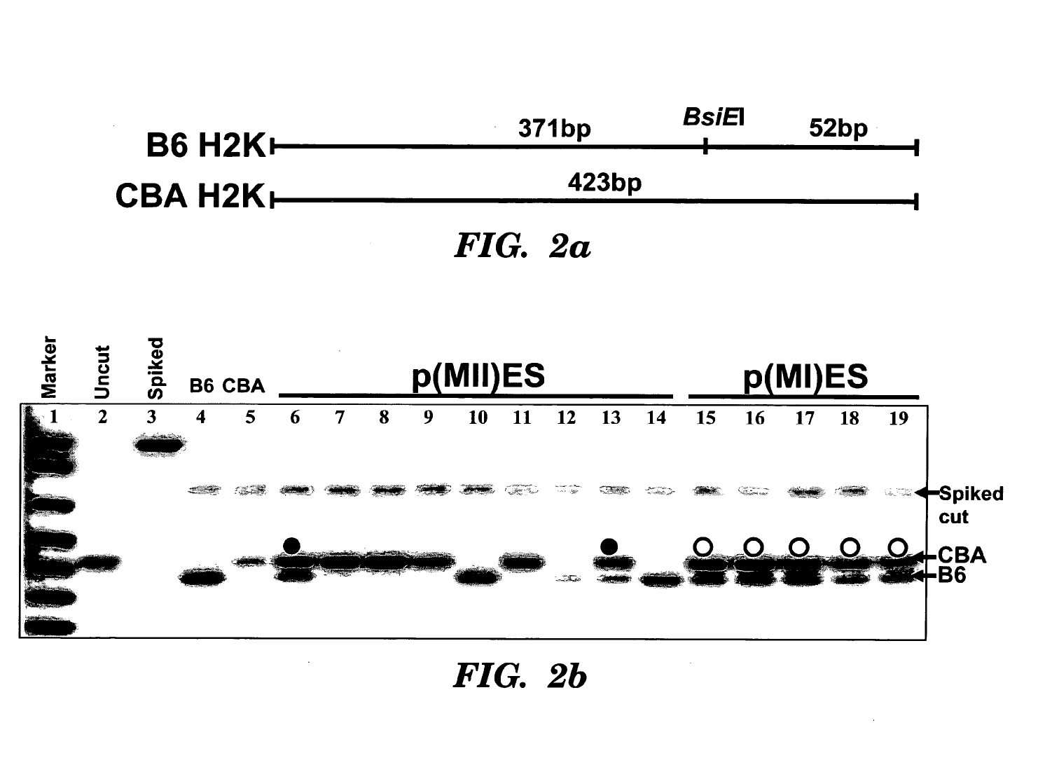

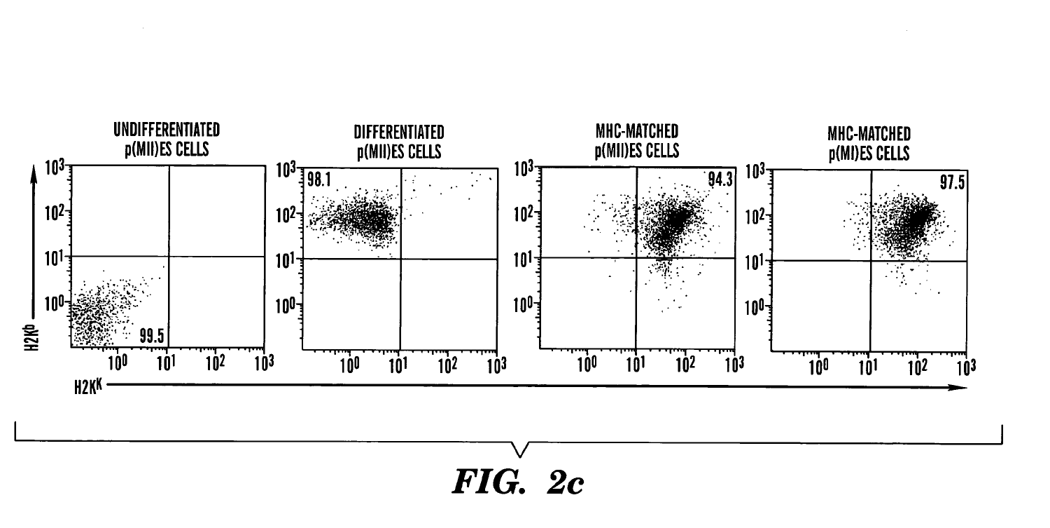

Artificially activating an oocyte after inhibiting the completion of meiosis II by cytochalasin B will result in a diploid parthenogenetic genome with considerable homozygosity. However, we reasoned that recombination events occurring between paired homologous chromosomes in meiosis I would produce progeny that had restored heterozygosity at the MHC loci (FIG. 1b). Recombination frequencies place the H-2 MHC locus at ˜18.5 centimorgans (cM) from the centromere on mouse chromosome 17 (11), thus predicting that approximately 1 in 5 of meioses should yield a cross-over event. We collected mature oocytes from six-week old C57BL/6×CBA F1 mice, and initiated parthenogenetic embryo development by activation with calcium ionophore and incubation with cytochalasin B and 6-dimethylaminopurine, a protocol that prevents extrusion of the second polar body and promotes oocyte maturation. From the 74% of activated oocytes that developed to blastocysts, we isolated 72 p(MII)ES cell lines (Table 1a). We employed PCR amplification followed by allele-specific restriction enzyme digestion of a single nucleotide polymorphism (SNP) within the H-2 region of chromosome 17 to determine if recombination had restored heterozygosity at the MHC loci (12). Two out of nine randomly selected p(MII)ES lines indeed demonstrated both maternal alleles (FIG. 2b; black circles). We call ES cells selected in this manner histocompatible (h-) p(MII)ES cells. The h-p(MII)ES cells were differentiated into embryoid bodies (EBs) for 14 days followed by culture on gelatin-coated tissue culture plates for an additional 14 days in order to examine MHC antigen expression on a differentiated population of epithelial cells (13). Differentiated derivatives of p(MII)ES cells that had not recombined at the MHC loci by polymorphism analysis expressed only one of the parental MHC proteins (H2K^{b}), whereas the h-p(MII)ES cells that had recombined expressed both H2K^{b} and H2K^{k} on all cells (FIG. 2c). These data confirm the heterozygous genotype by MI-IC antigen expression, and eliminate the possibility that the heterozygosity is an artifact of the admixture of homozygous p(MII)ES cells. Quantitative flow cytometric analysis of DNA content, as determined by staining with the Hoechst 33342, and direct chromosome counting of selected cell lines confirmed a normal diploid DNA content in the h-p(MII)ES cells. Additional heterozygosity analysis is presented below.

Isolation of Histocompatible (h-) p(MI)ES Cells.

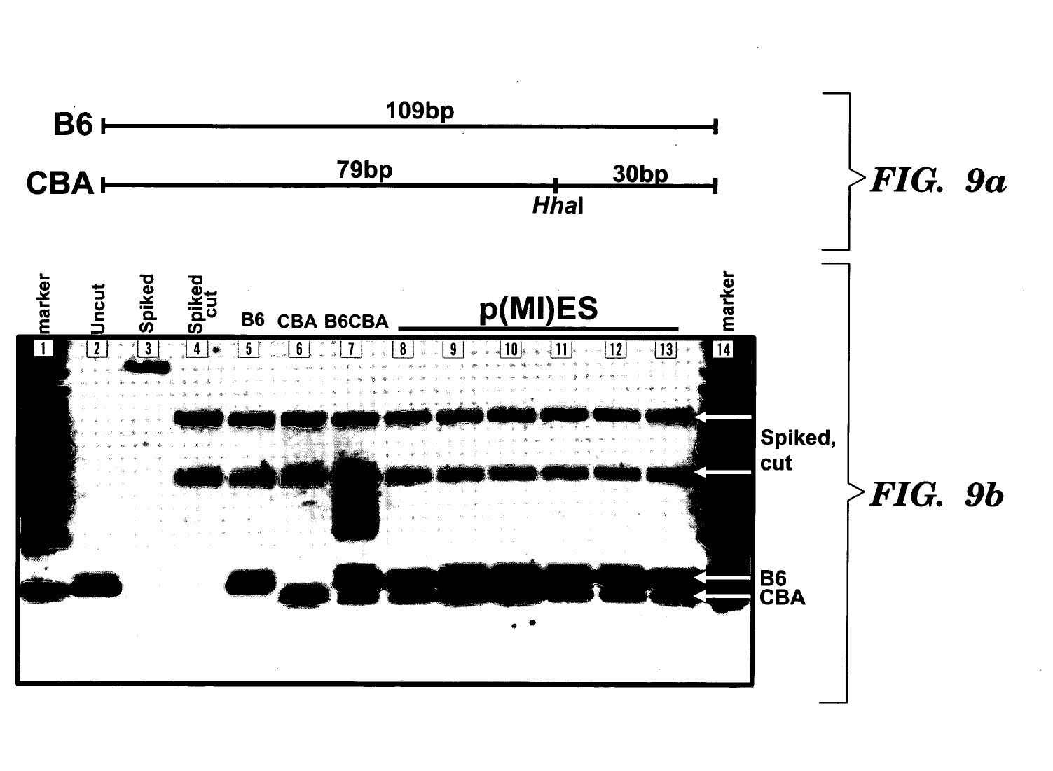

In a second method aimed at generating genetically matched p(MI)ES cells, we induced parthenogenetic development of immature oocytes while interfering with the segregation of the paired homologous chromosomes during metaphase I of meiosis (MI). This protocol prevents segregation of the maternal and paternal genomes and has been reported to yield parthenogenetic embryos that are genetic clones of the oocyte donor (FIG. 1c) (14). We collected immature oocytes from C57BL/6×CBA F1 mice 7-9 hours after superovulation with human chorionic gonadatropin and induced parthenogenetic development by incubation with cytochalasin D followed by activation of the oocytes by calcium ionophore. From the 56% of activated oocytes that developed into blastocysts we isolated 23 ES cell lines (Table 1b). MHC genotyping was performed on 5 randomly selected ES cell lines using the method described above. All 5 ES lines were heterozygous at the MHC region (FIG. 2b; white circles). Representative ES cells were differentiated for 28 days in culture as described above, and examined by flow cytometry. The differentiated derivatives expressed both H2K^{b} and H2K^{k} on all cells, confirming heterozygosity of the MHC locus by surface protein expression (FIG. 2c). We likewise confirmed heterozygosity in all 6 lines at a distinct marker in the Tap1 gene region using a similar PCR amplification and restriction fragment length polymorphism strategy (FIG. 9). In 15 cell lines, we documented normal diploid chromosome content by direct chromosome counting or quantitative analysis of DNA content in cells stained by Hoechst 33342, whereas 8 lines showed variable chromosome content. We call ES cells isolated in this manner histocompatible (h-) p(MI)ES cells. A more detailed genomic analysis that distinguishes the diploid and aneuploid cells is presented below.

Analysis of Peri-Centromeric Genotype in h-p(MII)ES and h-p(MI)ES Cell Lines.

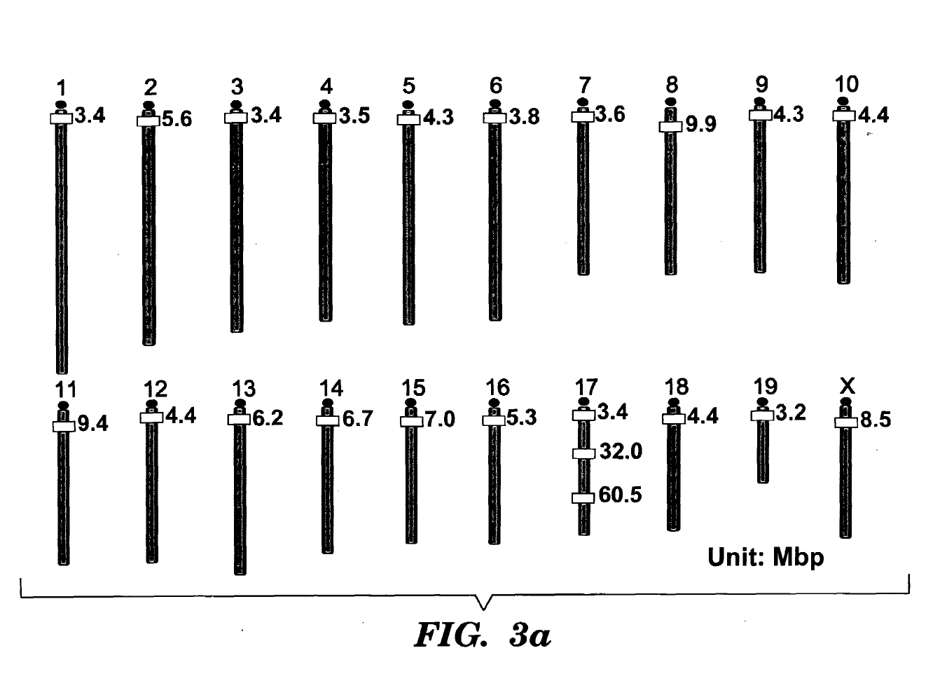

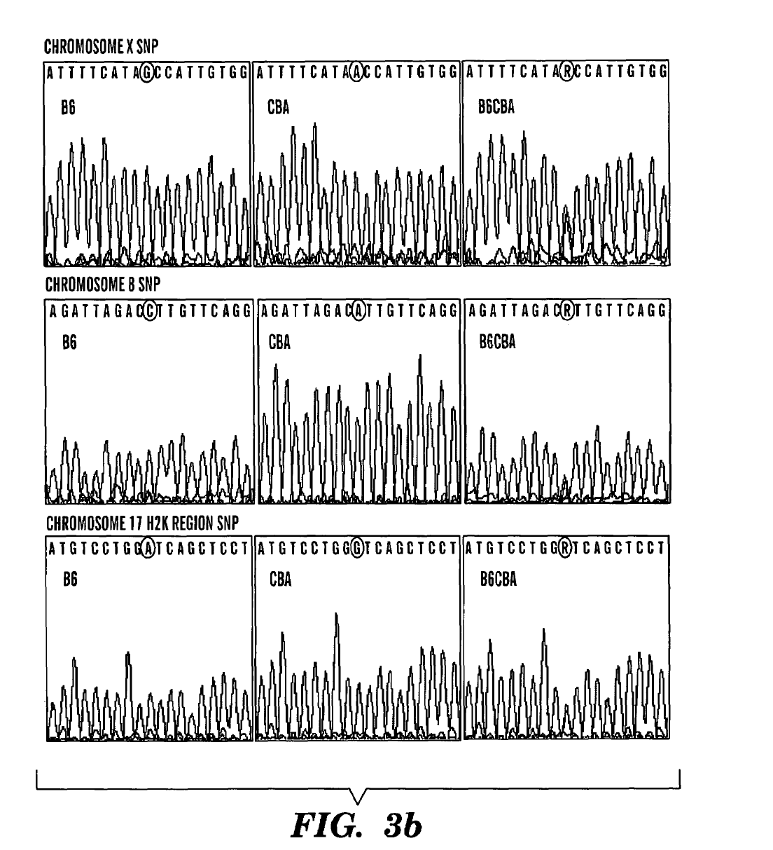

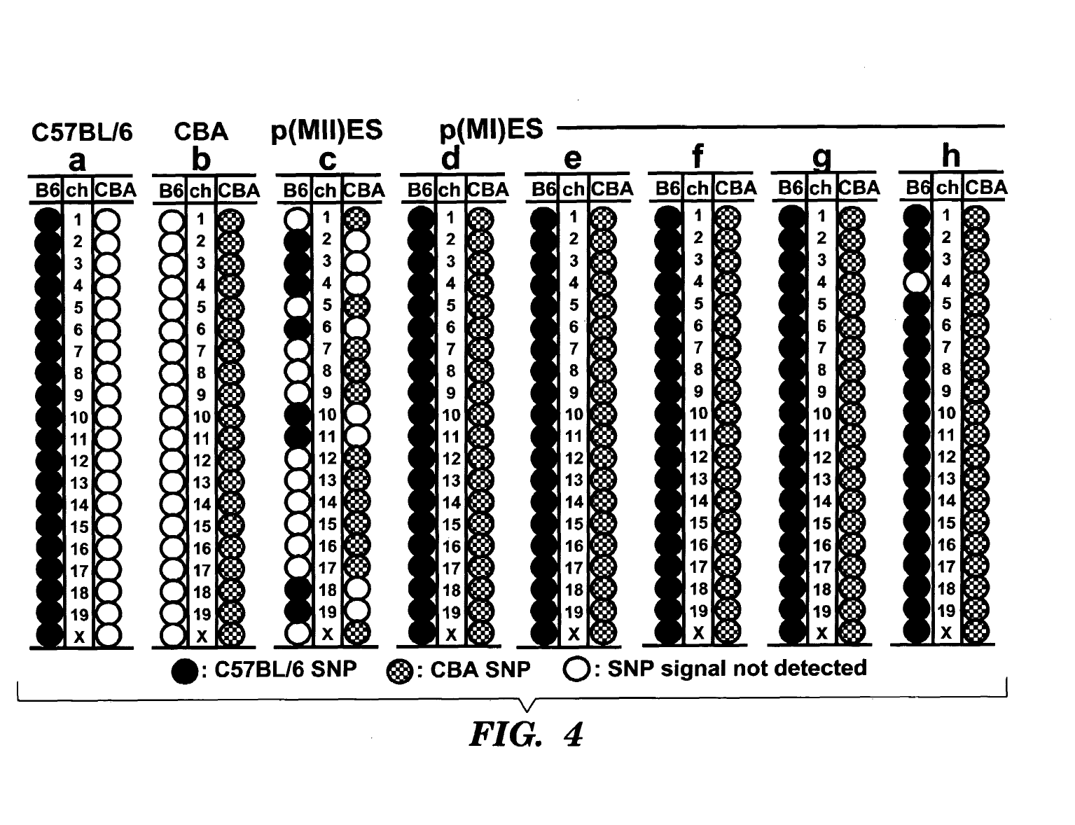

To confirm the expected pattern of chromosomal segregation induced under the different oocyte activation protocols, we sought to determine the peri-centromeric genotype of the h-p(MII)ES and h-p(MI)ES cell lines using SNPs that distinguish the parental mouse strains (C57BL/6 and CBA) (15) . We selected a SNP locus on each chromosome close to the centromere that should sustain minimal recombination (average distance, 5.5 Mbp; FIG. 3). The locus harboring the SNP was amplified by PCR and sequenced, which allowed us to distinguish C57BL/6, CBA, and B6CBAF1 specific profiles (data not shown). As hypothesized, all h-p(MII)ES cells were found to be homozygous for either the C57BL/6 or CBA SNPs (FIG. 4c), whereas all but one of the h-p(MI)ES cells were found to be heterozygous for all SNPs tested (FIG. 4h). Homozygosity of one locus in one h-p(MI)ES cell suggested that this line had lost one chromosome or that this locus had recombined during the process of parthenogenetic cloning.

Recombination Patterns of the p(MII)ES/p(MI)ES Cell Lines.

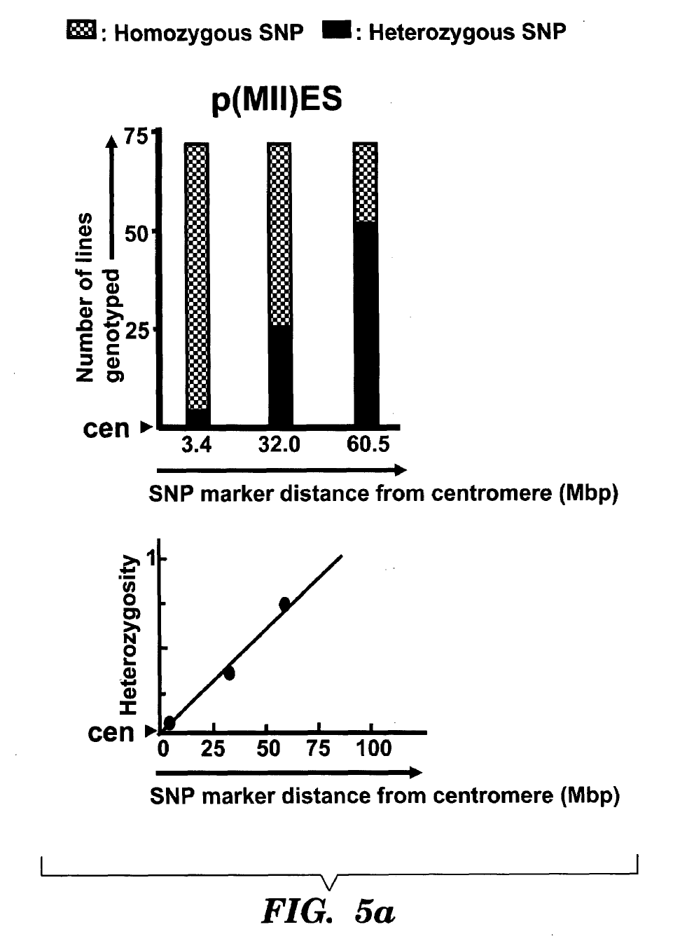

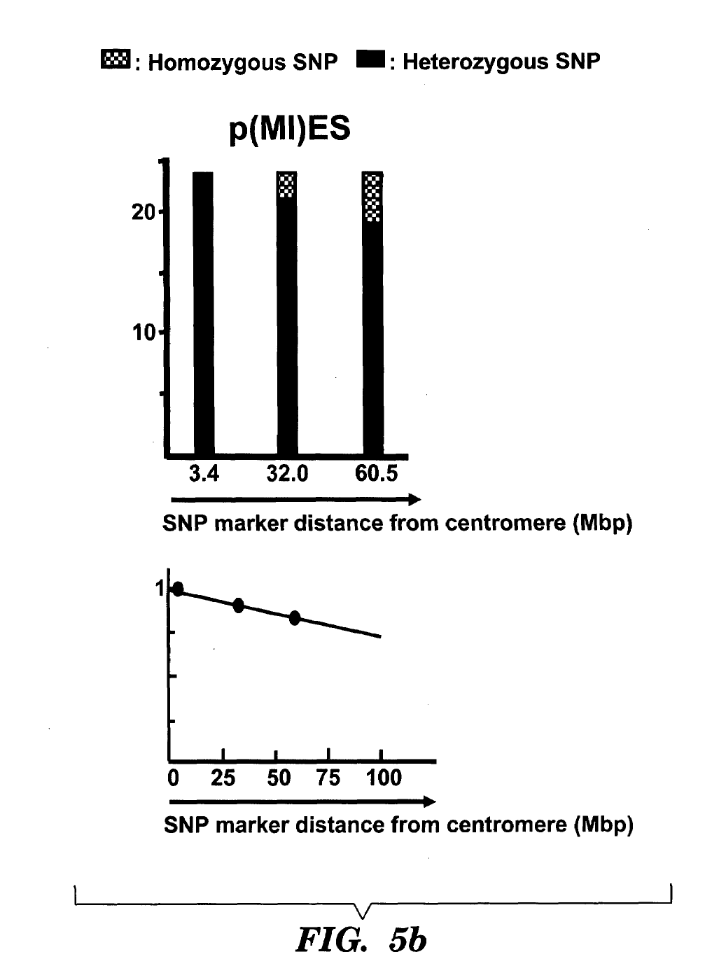

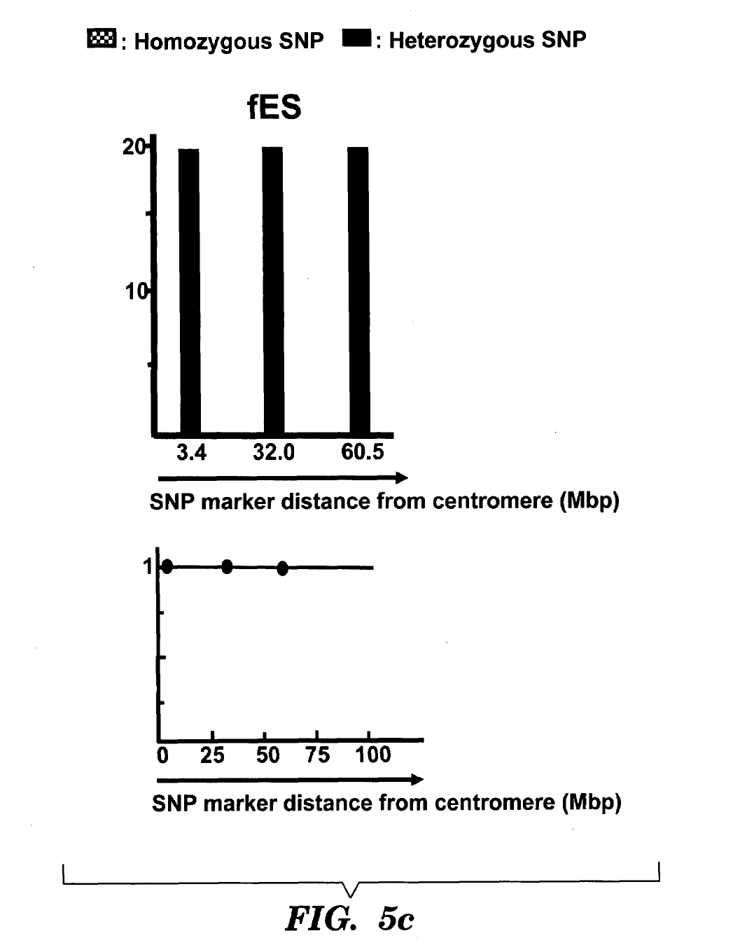

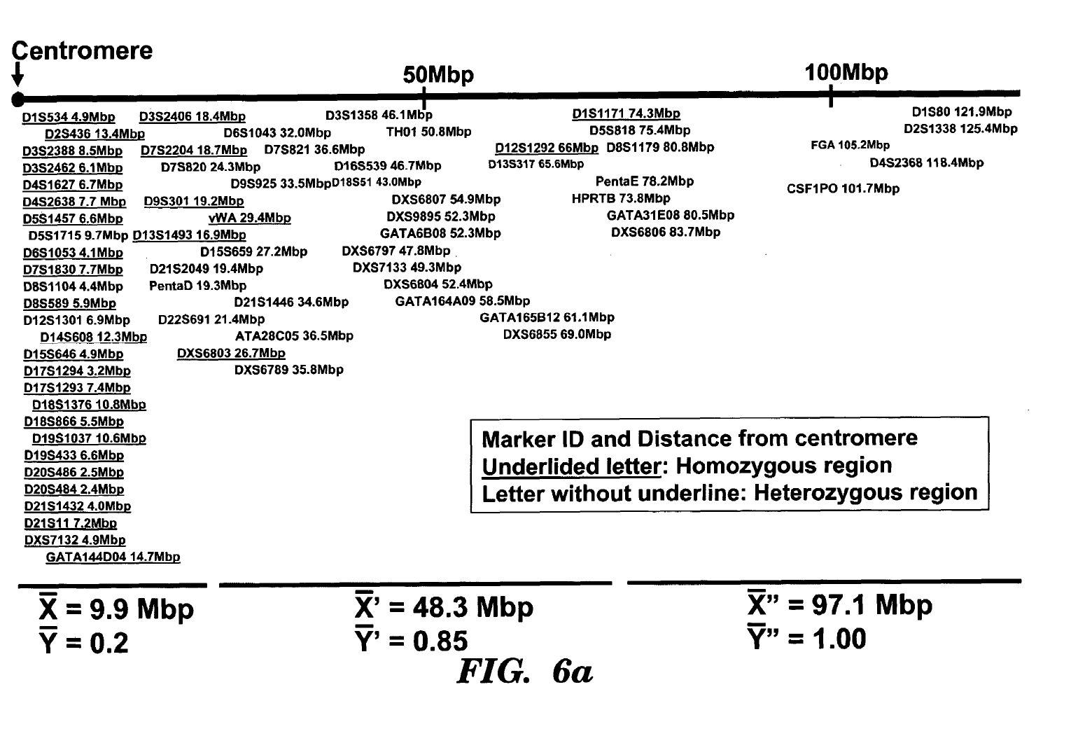

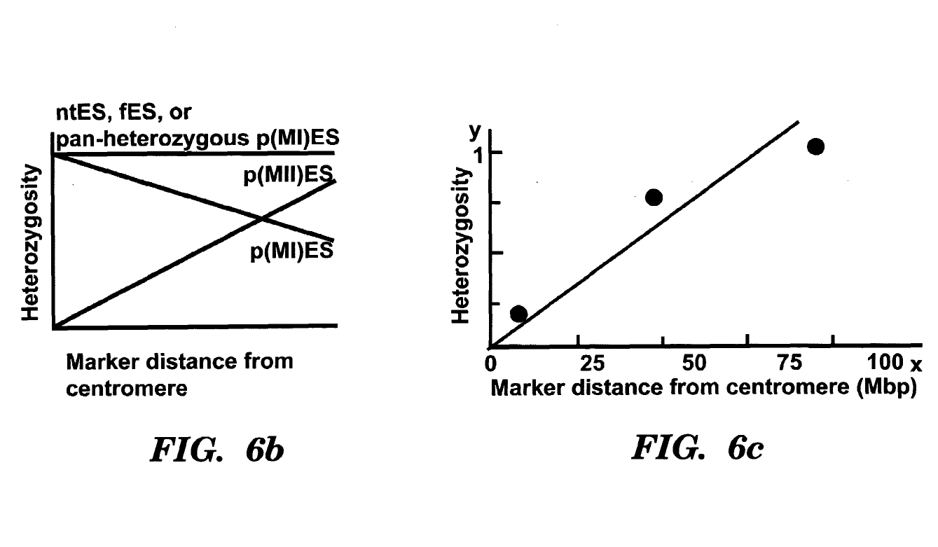

To further analyze the frequency and distribution of recombination events in p(MII)ES and p(MI)ES cells, we performed additional SNP genotyping at 3 loci located at 3.4 Mbp (4.0 cM), 32.0 Mbp (18.65 cM), and 60.5 Mbp (33.5 cM) from the centromere of chromosome 17. Given the nature of the two distinct protocols for parthenogenetic activation, we reasoned that h-p(MII)ES cells would be predominantly homozygous, with recombination reflected by a telomeric predominance of heterozygous SNPs. A survey of 72 independent clones of h-p(MII)ES cells indeed confirmed that the frequency of heterozygous SNPs increased in proportion to the distance from the centromere to the SNP (FIG. 5a). Conversely, we reasoned that the p(MI)ES cells would be predominantly heterozygous, with recombination reflected by a telomeric predominance of homozygous SNPs. In a survey of 23 p(MI)ES cell lines, the frequency of homozygous SNPs increased in proportion to the distance from the centromere to the genetic markers (FIG. 5b). ES cells isolated from embryos that result from natural fertilization events between strains of inbred mice (fES cells from F1 matings) should show heterozygosity at all loci, because the gametes derive from homozygous parents in which meiotic recombination is genetically invisible. As anticipated, we detected no homozygosity at the three SNP loci on chromosome 17 in 20 fES cell lines (FIG. 5c). Therefore, by plotting the heterozygosity rate vs. marker distance from the centromere, we can readily determine whether an ES cell represents the p(MI)ES, p(MII)ES, or fES type (FIG. 6b).

Assessment of Recombination Patterns and Frequency by Genome-Wide SNP Genotyping

The low resolution SNP genotyping performed by PCR amplification and sequencing of a few loci generally supported our expected pattern of recombination in the p(MII)ES cell lines: predominant homozygosity in the p(MII)ES cells with recombination at the distal ends of chromosomes and global heterozygosity in the p(MI)ES cells. Our observation of a low level of recombination in the p(MI)ES cells suggested that either all p(MI)ES cells sustained infrequent recombination, or that the p(MI)ES isolation protocol generated a mixture of cells, some with recombination but others that contained the complete genetic complement of the oocyte donor and were effectively genetic clones. We therefore genotyped a standard panel of 768 mouse markers located across the genome in 17 p(MII)ES and 20 p(MI)ES cells (an expansion of a previously described SNP set) (16). A total of 514 markers spanning the 2.25 Gb across the 19 autosomes were informative: they were polymorphic between B6 and CBA and had a control F1 correctly called as heterozygous (resulting in an average inter-marker distance of 4.6 Mb).

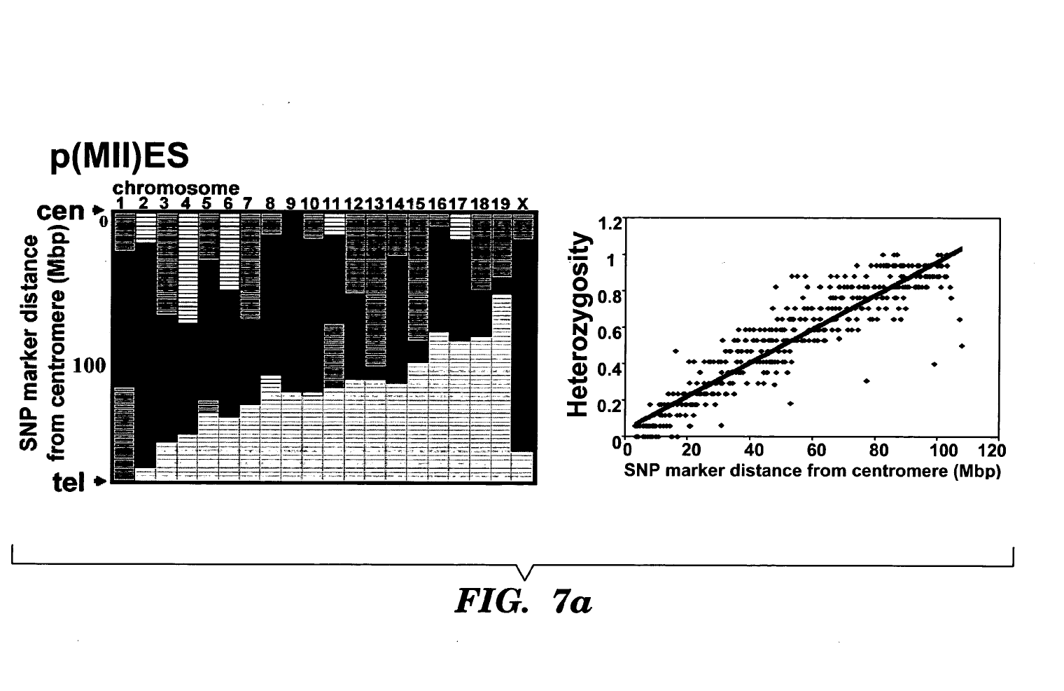

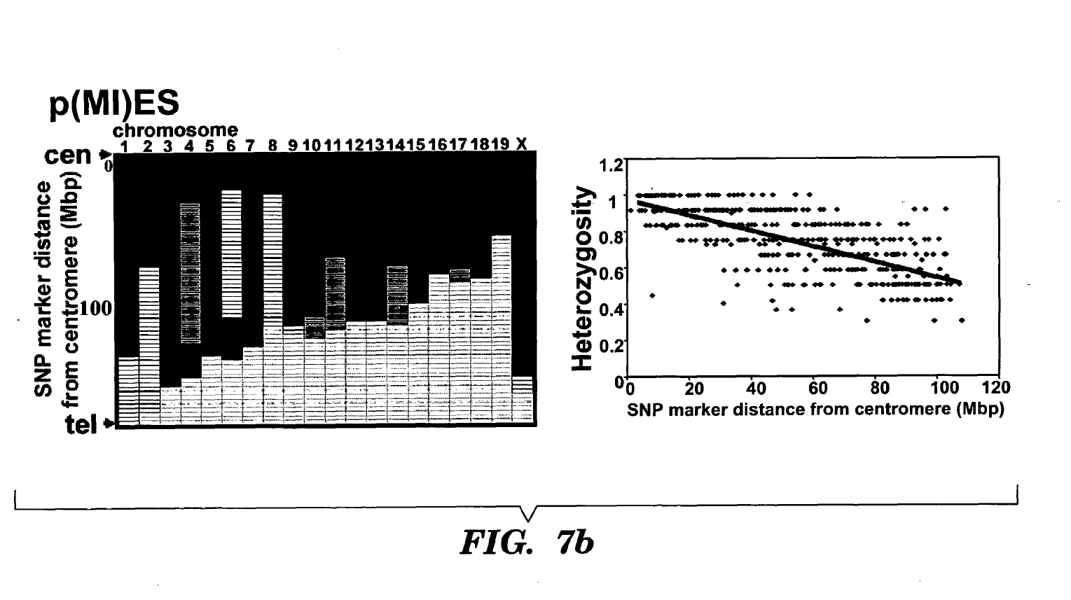

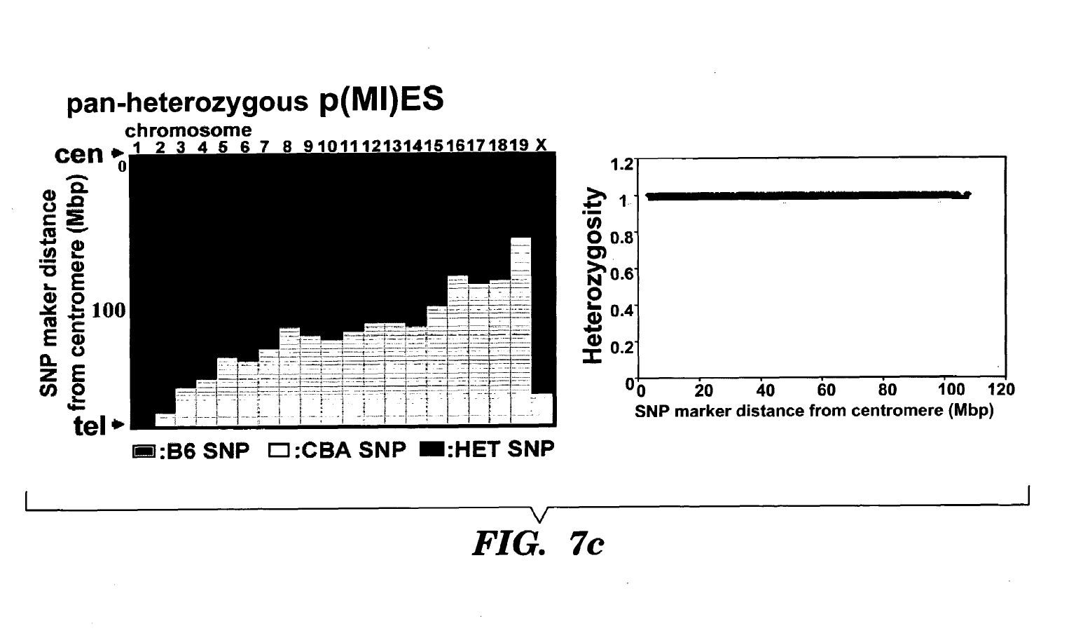

The results of the higher resolution SNP genotyping confirmed the expected patterns and frequency of recombination for the p(MII)ES cells. Because of the disruption of sister chromatid segregation in MII, all chromosomes were substantially homozygous beginning at their centromeres and extending distally towards the chromosome ends (FIG. 7a). Heterozygosity of SNP markers increased in frequency in proportion to the genetic distance from the centromere (FIG. 7a). Some cells harbored genotypes that showed homozygosity of B6 SNPs near the centromere, followed by a region of heterozygosity, and then a telomeric region of homozygosity of CBA SNPs (N.B.: chromosomes 8 and 10 in FIG. 7a). In the parthenogenetic protocol, this pattern is consistent with meiotic recombination between both sister chromatid pairs of homologous chromosomes, which occurs during MI, followed by segregation of the bivalents into separate cells upon extrusion of the first polar body, and then co-segregation of the recombinant sister chromatids into the same cell due to inhibition of extrusion of the second polar body (FIG. 1b). With both sister chromatids undergoing recombination separately, we postulated that p(MII)ES cells, while constrained to be homozygous at the centromeres, would manifest recombination at a rate that would be equivalent to an F2 offspring of two F1 mice. We calculated the genetic linkage map from the p(MII)ES genotypes using MAPMAKER/EXP v3 under the model of an F2 and, across all autosomes, estimated a total map length of 1329 cM. This is broadly consistent with the MIT/Whitehead map which reported a total autosomal map length estimate of 1338 cM (17). Due to recombination, approximately 33% of our cohort of 72 p(MII)ES cells had indeed regenerated the maternal MHC genotypes on both chromosomes.

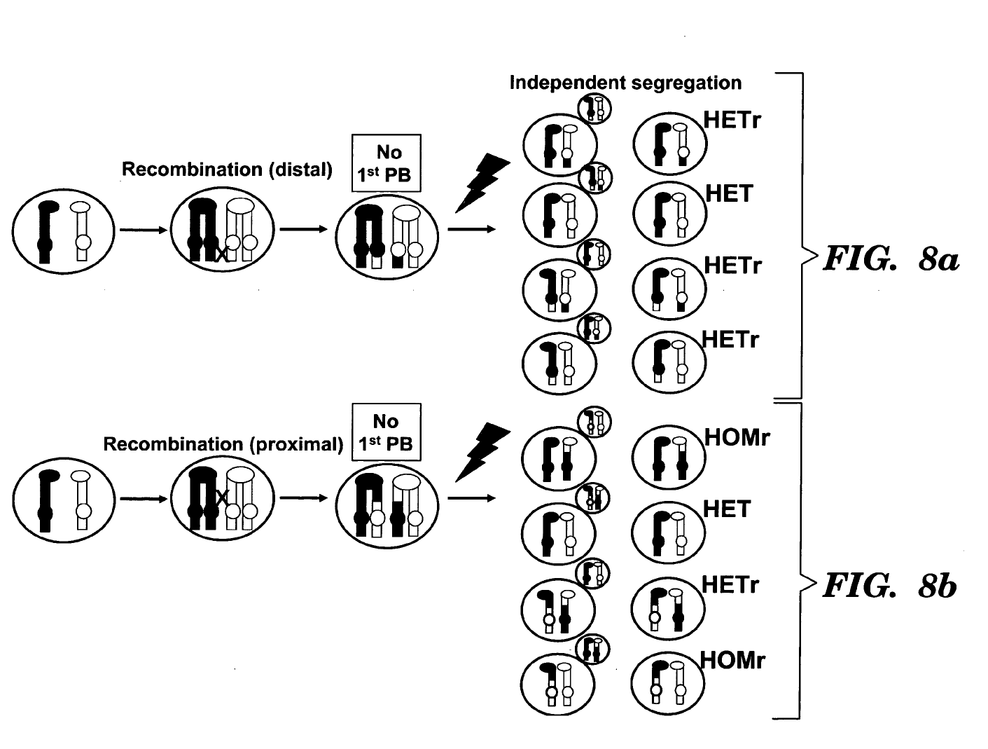

Analysis of the recombination patterns of 20 p(MI)ES cells showed two distinct sub-groups. 12 of the p(MI)ES cells showed a predominant pattern of heterozygosity beginning at the centromere followed on some chromosomes by telomeric regions of homozygosity. This occurs because the protocol interferes with completion of MI (by blocking extrusion of the first polar body), thereby impeding independent segregation of the recombinant chromosomes into separate cells. With further oocyte maturation and activation of mitotic cleavage, the recombinant sister chromatids ultimately segregate independently into the daughter cells (as illustrated in FIG. 8). When the genotype data of these 12 lines was similarly evaluated under the assumption of an F2 intercross, the observed map length was significantly suppressed (910 cM). Despite the high density of polymorphic markers examined, 94 of the 228 autosomes in these 12 lines were observed to be completely heterozygous. These observations drive the estimated map length down compared to the p(MII)ES and a standard F2 recombination pattern. These data can be reconciled because in many cases the recombinant chromosomes will co-segregate by chance into the same cell, thereby preserving heterozygosity in the genotyping assay (FIG. 8).

A subset of 8 p(MII)ES cells demonstrated complete heterozygosity of the entire genome across all loci, suggesting genetic identity with the oocyte donor. Because the immature oocytes subjected to parthenogenetic activation are not perfectly synchronized, we speculate that the protocol can occasionally produce a parthenogenetic clone by one of two postulated mechanisms: 1) interference with recombination events that would normally accompany completion of MI because of disruption of karyokinesis (blockade of extrusion of the first polar body), resulting in a tetraploid cell that retains all of the maternal chromosomes obligate co-segregation of the recombinant chromosomes into the same daughter cell. Of note, direct counting of chromosomes revealed a normal diploid number 40 for a subset of p(MI)ES cells (n=3), but aneuploidy of each of the 8 fully heterozygous cloned p(MI)ES (pc(MI)ES) cells (range of chromosomes 38-77), consistent with infidelity of chromosomal segregation during the process of artificial oocyte activation in some embryos.

Maternal Imprint Status in p(I)ES Cells.

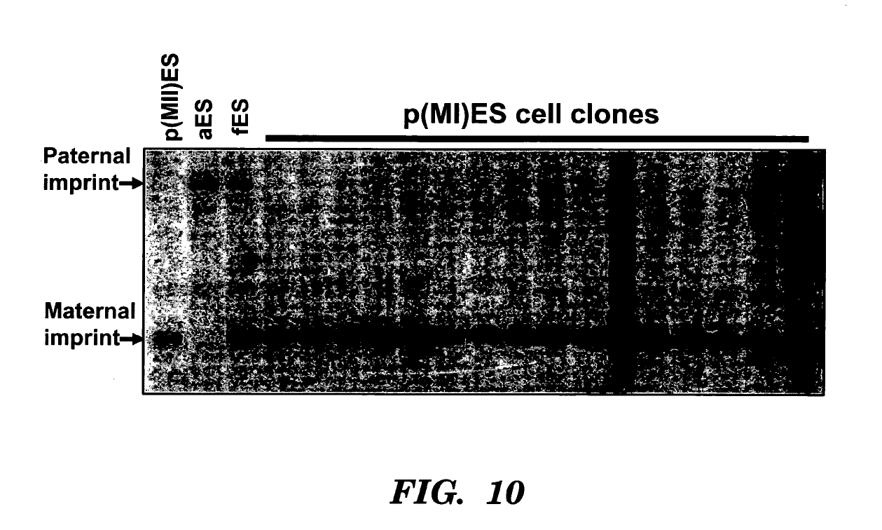

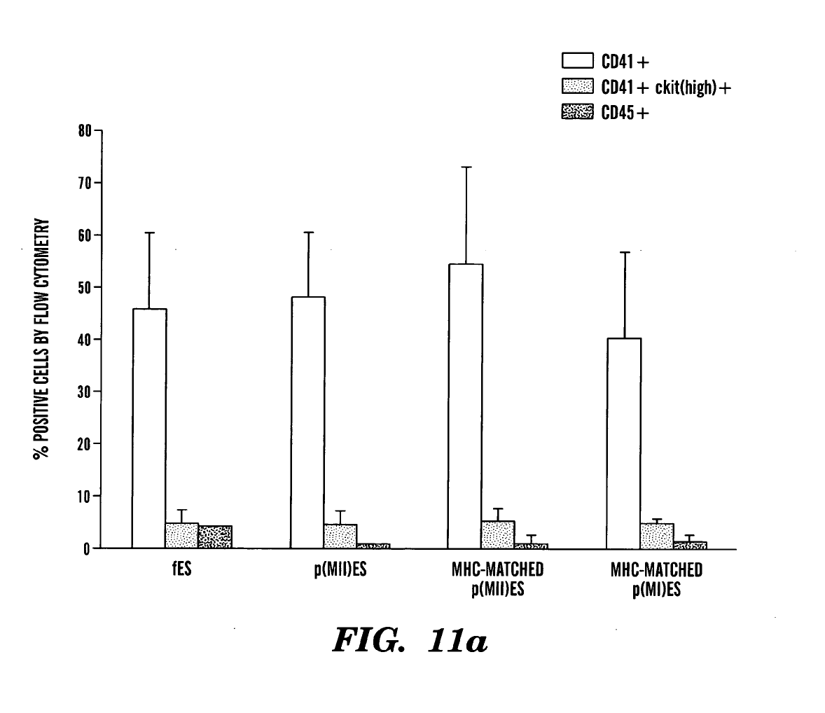

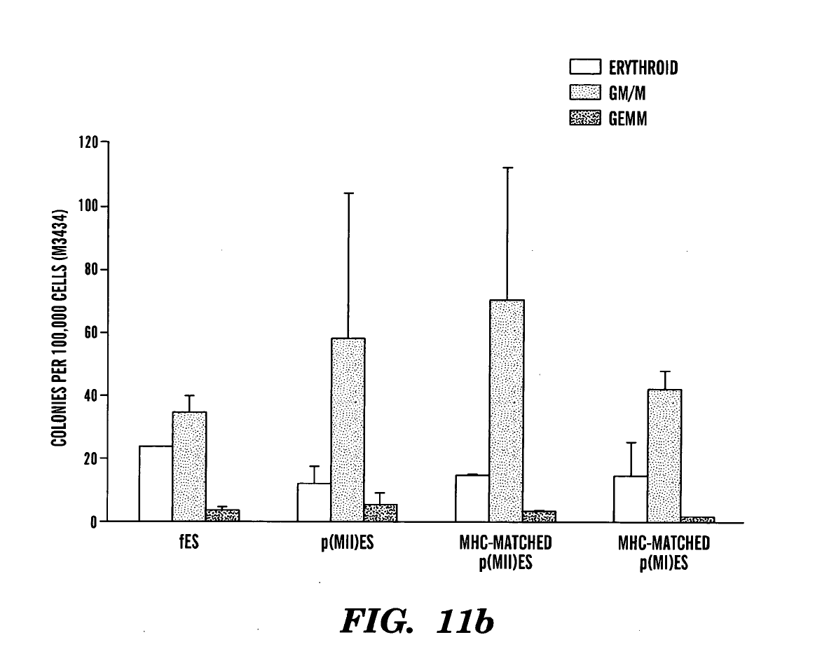

During germ cell development, the unique parental methylation marks, or imprints, are first erased in the primordial germ cells and later re-established during oogenesis or spermiogenesis so that specific loci are expressed from either the maternally or paternally inherited allele. Because p(MII)ES cells are established after the re-establishment of imprints in the growing oocyte (18), p(I)ES cells should lack paternal imprints and carry only maternal methylation marks on both chromosomes. We used the methylation sensitive restriction endonucleases PstI/NotI to digest genomic DNA isolated from p(MII)ES, p(MI)ES, and androgenetic ES cells (aES, derived from blastocysts developed from two sperm pronuclei) (19) to distinguish the allele that carries paternal vs. maternal methylation marks. The p(MII)ES cells showed only the maternal allele; the aES cells only the paternal allele, while fES cells show both. All p(MI)ES cells showed only the maternal allele (FIG. 10), consistent with their derivation from cells that harbor only a maternal genome.

Differentiation potential of h-p(MI)ES cell lines.

In order to assess the pluripotency of the h-p(MII)ES and h-p(MI)ES cells, we evaluated their differentiation potential by in vitro and in vivo assays. We injected two million cells subcutaneously into immune-deficient mice and observed robust teratoma formation from multiple h-p(MII)ES, h-p(MI)ES, and fES cell lines. Histology of teratomas revealed tissue elements of all three embryonic germ layers for each class of ES cell: mesoderm (bone, bone marrow, muscle, and cartilage), endoderm (respiratory epithelium, exocrine pancreas), and ectoderm (brain, melanocyte (iris), and skin (data not shown). Using previously published methods for in vitro differentiation of ES cells, we observed rhythmic contractility in embryoid bodies (EBs) consistent with cardiomyocyte development, and comparable numbers of hematopoietic elements as measured by methylcellulose-based colony forming cell assays and flow cytometric analysis for the hematopoietic markers ckit+, CD41+, and CD45+(20) (FIG. 6a, FIG. 6b). We generated chimeric mice by injecting h-p(MII)ES and h-p(MI)ES cells into recipient blastocysts. Examples of h-p(MII)ES and h-p(MI)ES cells each demonstrated fetal liver chimerism and high-level skin chimerism of adult mice (data not shown). No germ line transmission of gametes from the h-p(MII)ES or h-p(MI)ES cells was noted in 8 matings of female chimeras that generated more than 700 progeny. Moreover, injection of over 50 tetraploid embryos with h-p(MI)ES cells failed to result in live births, consistent with the known developmental limitation of parthenogenetic mouse embryos (1). Although not fully competent to sustain organismal development due to a lack of paternal imprints, h-p(MII)ES and h-p(MI)ES cells appear to share a comparable degree of multi-lineage tissue differentiation as fES cells.

Histocompatibility of Differentiated Progeny of h-p(MII)ES and h-p(MI)ES Cells.

In order to determine whether the MHC-matched pES cells would be accepted as tissue transplants in recipient mice, we injected undifferentiated C57BL/6J×B6CBA F1 fES and selected h-p(MII)ES and h-p(MI)ES cells subcutaneously into immunodeficient as well as MHC matched and mis-matched immunocompetent recipients. When immunodeficient mice (Rag2^{-/-}/γc^{-/-}) were injected with either 5×10⁵ cells or 2×10⁶ cells, we observed teratomas in all mice with all cell lines within 6 weeks (n=7 for each cell type). In contrast, no teratomas formed when any of the cell lines were injected into immunocompetent C57BL/6, B6CBA, or CBA recipient mice (n=7 each; Table I). Like early embryonic tissues (4, 21), undifferentiated mouse ES cells do not express MHC antigens (FIG. 2c), which render them susceptible to rejection by NK cells (4). MHC antigen expression can be detected after differentiation of mouse ES cells (FIG. 2c and data not shown). Therefore, we differentiated C57BL/6J×B6CBA F1 fES, h-p(MII)ES, and h-p(MI)ES cells into EBs for two weeks, then injected 10⁶ cell equivalents as whole EBs subcutaneously into recipient mice of several MHC matched and mis-matched genotypes. Differentiated fES cells resulted in teratomas when injected into genetically identical and immunocompromised recipient mice. However, the heterozygous fES cells failed to form teratomas in homozygous C57BL/6 or CBA recipients. Based on our analysis of the MHC locus in h-p(MII)ES and h-p(MI)ES cell lines, we would expect these cells to behave like heterozygous fES cells. As predicted, differentiated h-p(MII)ES and h-p(MI)ES cells formed teratomas in heterozygous matched and immuno-compromised recipient mice, but were rejected in homozygous recipients (Table 2). These data confirm the histocompatibility of differentiated tissues from the pES cells that had been selected for genetic identity at the MHC loci.

We provide two methods for parthenogenetic activation of oocytes that enable us to isolate pluripotent murine ES cells that are genetically matched to the oocyte donor at the MHC loci. By applying genotyping analysis to pES cells isolated from the activated oocytes of a hybrid mouse (C57BL/6×CBA), we have selected lines in which specific meiotic recombination events and inheritance of the relevant sister chromatids have restored the maternal MHC genotype. When these genetically defined pES cells are pre-differentiated into EBs prior to injection into immunocompetent recipient mice, the tissue engrafts as long as there is MHC identity between the donor cells and the recipient mouse. These data demonstrate that selected parthenogenetic ES cells can provide a source of histocompatible tissues for transplantation.

The traditional method for experimental parthenogenesis in mice entails activation of oocytes that are arrested at the second meiotic metaphase, concurrent with cytochalasin treatment to block completion of the second meiotic division. The reduction to haploidy that would normally accompany the extrusion of the second polar body fails to occur, diploidy is maintained, and mitotic cell division ensues. The resulting embryo is largely homozygous, except for regions that have reverted to heterozygosity because of recombination events during MI. Thus, parthenogenetic recombinant, or h-p(MII)ES cells, can be selected so that their MHC genotype will match that of the oocyte donor. Activation of immature oocytes and inhibition of the first meiotic division in an attempt to isolate parthenogenetic clones ensures that heterozygosity is preserved across the genome, except for those regions that convert to homozygosity because of recombination. The h-p(MI)ES cells retain significant genetic identity with the oocyte donor and likewise can be selected for genetic identity at the MHC or any other loci. Moreover, all forms of pES cells retain the mitochondrial genome of the oocyte donor, unlike genetically matched ES cells that are created by nuclear transfer into oocytes from an unrelated donor. A subset of h-p(MI)ES cells retains complete heterozygosity at all loci, suggesting genetic identity with the oocyte donor. However, we speculate that such parthenogenetic clones arise from artificial activation of immature tetraploid oocytes and random chromosome loss due to aberrant chromosomal segregation events. These cells manifest significant aneuploidy, thus betraying their identity as true clones and calling in to question their value as a source of tissues for transplantation.

Enucleation sometimes fails when attempting to derive donor-specific ES cell lines via nuclear transfer, leaving open the possibility for development of a parthenogenetic embryo. The status of maternal or paternal specific imprint genes can be monitored to identify parthenogenetic ES cells. However, the data presented here demonstrates that discerning the distinct patterns of homozygosity and heterozygosity in ES cell lines through SNP genotyping across the genome provides another means to determine whether lines are the result of parthenogenesis, nuclear transfer, or natural fertilization. If homozygosity predominates near the centromere and heterozygosity is observed with increasing frequency at telomeric loci, then a p(MII)ES cell line has been derived by parthenogenetic activation of the oocyte following completion of the first meiotic segregation of homologous chromosomes. However, if heterozygosity predominates near the centromere with increasing frequency of homozygosity at markers distal to the centromere, then a p(MI)ES cell line has resulted from a disruption in segregation of the homologous chromosomes that normally occurs in MI, followed by centromere separation and sister chromatid segregation into diploid progeny during the artificial activation and oocyte maturation process. A cell line derived from an embryo produced by nuclear transfer from a somatic cell should for the most part be a complete genetic match of the nuclear donor, as only rare occurrences of mitotic recombination would alter the expected pattern of heterozygosity. Furthermore, there should be no discernable pattern of heterozygosity relative to centromeric distance. Similarly an ES cell line derived from a fertilized blastocyst should be a combination of sperm and egg donor haplotypes, again with no relationship between frequency of heterozygosity of markers and distance from the centromere.

Parthenogenetic ES cells have been isolated from mice and primates (2, 3) and p(MII)ES and p(MI)ES cells can be isolated from human embryos. Indeed, based on applying the analysis outlined above to publicly available SNP genotyping data for the NT-1 cell line reputed to be the first human ES cell line derived by nuclear transfer (22), we conclude that this cell indeed represents a p(MII)ES cell line. The Seoul National University Investigation Committee (SNUIC) released preliminary DNA fingerprint analyses data of NT-1 ES cells in January, 2006 (23), indicating that 8 of 48 markers were homozygous and shared with the oocyte donor, raising the possibility that the line represented the accidental isolation of a p(MII)ES cell due to enucleation failure. In May, 2006, the SNUIC released DNA fingerprinting data on an additional 71 markers, as well as an analysis of the imprint status of the H19, KCNQ1OT1, and SNRPN genes in NT-1 ES cells, which showed a maternal pattern consistent with parthenogenesis (24). When the DNA genotyping data is arranged according to marker distance from the centromere, a clear pattern of homozygosity at markers located proximal to the centromere and increased heterozygosity at more distal markers is apparent (FIG. 6a). When we plot the rate of SNP heterozygosity vs. marker distance from centromere, we observe the characteristic pattern of p(MII)ES cells (FIG. 6b, FIG. 6c). This analysis indicates that the NT-1 ES cell represents the first example of a human p(MII)ES cell.

Here we demonstrate a means of exploiting parthenogenesis and genetic recombination to isolate pluripotent murine embryonic stem cell lines with genetically defined MHC loci that are critical to tissue transplantation. Applying similar methods to the derivation of human p(MII)ES and p(MII)ES cells offers a means to generate ES cells from women that could serve as a source of customized histocompatible tissues for transplantation. Methods exist for generating androgenetic embryos by reconstruction with two sperm nuclei (20), thus androgenetic ES lines (aES) can be selected for histocompatibility for transplantation applications in men. Previous studies have suggested that when pES cells are injected into blastocysts they fail to chimerize certain tissues like skeletal muscle yet contribute significantly to others like heart, liver, brain, spleen, blood, and lung (1). In our hands, mice generated by injection of blastocysts with pES cells show multi-tissue chimerism, and in vitro differentiation of pES cells demonstrates robust numbers of hematopoietic progeny, indicating that pES cells are pluripotent. Beyond providing pre-clinical models of ES cell based therapies, these experiments allow new insight into genetic recombination during parthenogenetic activation. Isolation of p(MII)ES cells followed by SNP genotyping provides a means of genetic mapping of loci for phenotypes that can be defined through the study of ES cells.

Tables

TABLE 1
Parthenogenetic oocyte activation and
ES Cell derivation from B6CBAF1 mouse
a, p(MII)ES cell derivation

| | Stage | | | | | |
|---|---|---|---|---|---|---|
| | | | | | Blasto- | p(MII)ES |
| | 1 cell | 2 cells | 4 cells | Morula | cyst | cells |
| Rate of | 150/150 | 125/150 | 117/150 | 115/150 | 111/150 | 72/111 |
| Development | 100% | 83% | 78% | 77% | 74% | 65% |

b, p(MI)ES cell derivation

| | Stage | | | | | |
|---|---|---|---|---|---|---|
| | | | | | Blasto- | p(MI)ES |
| | 1 cell | 2 cells | 4 cells | Morula | cyst | cells |
| Rate of | 112/112 | 87/112 | 81/112 | 75/112 | 63/150 | 23/63 |
| Development | 100% | 78% | 72% | 67% | 56% | 37% |

TABLE 1 Legend. Parthenogenetic oocyte activation and ES cell derivation from B6CBA F1 mouse. Table 1a, Efficiency of p(MII)ES cell derivation. Hybrid B6CBAF1 mice (C57BL/6×CBA; Jackson Laboratories) were used as oocyte donors. Eight to ten week old female mice were superovulated by injection of 5 IU Pregnant mare serum gonadotropin (PMSG, Calbiochem 367222) followed 48 h later by injection of 5 IU Human chorionic gonadotropin (hCG, Calbiochem230734). Oocytes were collected 14-15 hours after hCG injection. Oocytes with cumulus cells were activated in KSOM (Specialty Media, MR-106-D) containing 10 μM calcium ionophore A23187 (Sigma, C7522) for 5 min in air, then in 2 mM 6-dimethylaminopurine (6-DMAP) (Sigma, D2629) and 5 μg/ml of cytochalasin B (Sigma, C6762) dissolved in KSOM at 37° C. in 5% CO2 for 3 hours. Embryos were then washed five times in 500 micro liters of KSOM. Embryos were cultured in KSOM. All cultures were performed at 37° C. in 5% CO2, 5% O2, and 90% N2. Two and four days after activation and culture, developmental stage was evaluated under a stereomicroscope. Thereafter, the zona pellucida of blastocysts was removed in 1% pronase in FHM media (Specialty Media, MR-024-D), and cells were cultured in the presence of mouse embryo fibroblasts (MEFs) in serum free ES maintenance media (Gibco, 10829-018) in 5% CO2, 5% O2, and 90% N2. Table 1b, Efficiency of p(MI)ES cell derivation. Mice were superovulated as above, but oocytes were collected from the ovary 9 hours after hCG injection. Cumulus cells were dispersed by incubation in hyaluronidase (Sigma, H4272: 1 mg/ml in KSOM) for 2-5 minutes at 37° C. for 5 min. Cumulus-free oocytes were then washed five times in 500 micro liter of KSOM. The cumulus cell free oocytes were incubated in KSOM containing 5 μg/ml of cytochalasin D (Sigma, C8273) for 3 hours. Cumulus-free oocytes were then washed five times in 500 micro liters of KSOM and incubated in KSOM at 37° C. in 5% CO2 for 6 hours. The oocytes were activated in KSOM containing 10 μM calcium ionophore A23187 for 5 min in air, then in 2 mM 6-dimethylaminopurine (6-DMAP) (Sigma, D2629) dissolved in KSOM at 37° C. in 5%. CO2 for 3 hours. Embryos were then washed five times in 500 micro liter of KSOM. All cultures were performed in culture condition at 37° C. in 5% CO2, 5% O2, and 90% N2 in serum free ES maintenance media, which greatly enhances ES cell isolation efficiency. Developmental stage was evaluated under a stereomicroscope.

TABLE 2
Teratoma formation following injection of h-p(MII)ES
and h-p(MI)ES cells into MHC matched and mis-matched
recipients (teratomas formed/total mice injected).

| | ES cell type | | | |
|---|---|---|---|---|
| | fES cells | fES cells | h-p(MII)ES | h-p(MI)ES |
| Recipient | from | from | cells | cells |
| mouse | C57BL/6 | B6 × CBA F1 | (B6/CBA) | (B6/CBA) |
| Rag2^{-/-} γc^{-/-} | + (3/4) | + (2/3) | + (3/3) | + (2/3) |
| CBA | − (0/4) | − (0/3) | − (0/4) | − (0/4) |
| C57BL/6 | + (2/4) | − (0/5) | − (0/5) | − (0/5) |
| B6 × CBA F1 | − (0/5) | + (1/5) | + (4/5) | + (4/5) |

TABLE 3
Primers used (Chromosome number: distance from
centromere (bp).

SNP PCR amplification primer set
Chr1: 3354789
5′- TGT TCC AGG TCA GTG ACT TCC -
3′
(SEQ ID NO: 1)
5′- GCC TTT TTC AAC TTG CCT CA -
3′
(SEQ ID NO: 2)
Chr2: 5609234
5′- CAA CAG AAA GGA GGC CAA AG -
3′
(SEQ ID NO: 3)
5′- TCA GTA CCA AGC ACG TGA GC -
3′
(SEQ ID NO: 4)
Chr3: 3750240
5′- TGC TTA TGG TTT TAT GTA ATA
GGC -3′
(SEQ ID NO: 5)
5′- TTT ATG CCA TGG TCC TTT GG -
3′
(SEQ ID NO: 6)
Chr4: 3476531
5′- ANA CAT GGA GCT CTC TGA
AAA TG -3′
(SEQ ID NO: 7)
5′- AGG AGG TGC AAT TCA GCT TT -
3′
Chr5: 4310159
(SEQ ID NO: 8)
5′- GCA ATT GCT GTT GAA AGC TG -
3′
(SEQ ID NO: 9)
5′- AAT GCC AAA CCC ATC CAT TA -
3′
(SEQ ID NO: 10)
Chr6: 3799842
5′- TGG TCC AAA TTT CCA TCA GC -
3′
(SEQ ID NO: 11)
5′- TGC CAG GCA TAT GGT TAG TG -
3′
(SEQ ID NO: 12)
Chr7: 3581601
5′- ATG GTT TGG GGG TAG AGG TC -
3′
(SEQ ID NO: 13)
5′- CCA CAA TAC TGA AGG GCA CA -
3′
(SEQ ID NO: 14)
Chr8: 9889063
5′- CCA CCA GCC TTT CCT AAA CA -
3′
(SEQ ID NO: 14)
5′- CCT CAA CCC AGA TCT CTC CA -
3′
(SEQ ID NO: 15)
Chr9: 4289768
5′- GGT CTC AAG CAG GTG AGC TT -
3′
(SEQ ID NO: 16)
5′- TTC TCA AAA TCT TTT TGG ATG
C -3′
(SEQ ID NO: 17)
Chr10: 4381768
5′- GAG GGA CTC ACA AGC CAC AT -
3′
(SEQ ID NO: 18)
5′- CCT TCT GGC CTT CTG TGA AC -
3′
(SEQ ID NO: 19)
Chr11: 9447523
5′- CTC ATT TGG AGG CCT CTG TC -
3′
(SEQ ID NO: 20)
5′- CCT TCT GGG TTC TGC TTC TC -
3′
(SEQ ID NO: 21)
Chr12: 4427782
5′- AAC TGG CCT AAG GGT CCA CT
3′
(SEQ ID NO: 22)
5′- CTG AGA CAT TGT CCC GCT TT -
3′
(SEQ ID NO: 23)
Chr13: 6192628
5′- TGT CTT CGC ACA TCT TGT CC -
3′
(SEQ ID NO: 24)
5′- TAA GCC AGC TTC TTC CAA GC -
3′
(SEQ ID NO: 25)
Chr14: 6685859
5′- GCT CCA GAA CCA AGA ACT GC -
3′
(SEQ ID NO: 26)
5′- AAA CAG CCT GAT CCC AAT GT -
3′
(SEQ ID NO: 27)
Chr15: 6955482
5′- AGA GTG GCC CAG AAA GTT CA -
3′
(SEQ ID NO: 28)
5′- CCA GCT CCA CTC CTA ACA GC -
3′
(SEQ ID NO: 29)
Chr16: 5321310
5′- TGG CAC ACT TGT GAC AAC CT -
3′
(SEQ ID NO: 30)
5′- GAT CTG ATG CTT CCC TGG AT -
3′
(SEQ ID NO: 31)
Chr17: 3383258
5′- CCC CTC CGA CAG AAC ATC TA -
3′
(SEQ ID NO: 32)
5′- GGT GAC AAG GGG TTT GAA GA -
3′
(SEQ ID NO: 33)
Chr17: 32 Mbp region (H2-K)
5′- cct ggg ctt cta ccc tgc t -3′
(SEQ ID NO: 34)
5′- CCA CCA CAG CTC CAG TGA G -
3′
(SEQ ID NO: 35)
Ch17: 60455318
5′- TGC TTA CCC TCA GCA AGA CA -
3′
(SEQ ID NO: 36)
5′- TTC TGG GTA GCT CAG GCT GT -
3′
(SEQ ID NO: 37)
Chr18: 4445867
5′- CAC AGA TGT CAG CTC CGT GA -
3′
(SEQ ID NO: 38)
5′- TTC CAA CTC CTC CAC TCA GG -
3′
(SEQ ID NO: 39)
Chr19: 3243816
5′- TCA TTC GGC CAA TAC ACA GA -
3′
(SEQ ID NO: 40)
5′- GGG AAA GGA CTT AGG CTT GC -
3′
(SEQ ID NO: 41)
ChrX: 8450263
5′- AAA TCA ACT CTT GCG GCT ACA -
3′
(SEQ ID NO: 42)
5′- CAA CAA AAT TTG GGG CTA
ACA -3′
(SEQ ID NO: 43)
SNP PCR sequencing primer
Chr1: 3354789
5′- CAC TGG AAG GAG GAA TTT CA-
3′
(SEQ ID NO: 44)
Chr2: 5609234
5′- CCT TTC ATG AAG CAG ATG
ACA -3′
(SEQ ID NO: 45)
Chr3: 3750240
5′- GCT GCT ACA AAT ACT AAG
AAA TGT TCC -3′
(SEQ ID NO: 46)
Chr4: 3476531
5′- GCT TTT GAT TTG TTG TCT TTT
TGA -3′
(SEQ ID NO: 47)
Chr5: 4310159
5′- CAG TTG GAA TGT GCA TCA GC -
3′
(SEQ ID NO: 48)
Chr6: 3799842
5′- TCA GCT ANA GCA TAT TAA TTC
AAA ACA -3′
(SEQ ID NO: 49)
Chr7: 3581601
5′- TAG GTG GGC ATG GGT GAG TA -
3′
(SEQ ID NO: 50)
Chr8: 9889063
5′- CCG AGC AGC TGG TAT TTG AT -
3′
(SEQ ID NO: 51)
Chr9: 4289768
5′- TGA TAA ATG AGA CCA CGA
TAC CA -3′
(SEQ ID NO: 52)
Chr10: 4381768
5′- CAG TGA AGA GCA CAC CCA TT -
3′
(SEQ ID NO: 53)
Chr11: 9447523
5′- GGC ATT GGC TAG ATA CAC
AAA A -3′
(SEQ ID NO: 54)
Chr12: 4427782
5′- TCA TTC TTT GCT GTG GAA ACA -
3′
(SEQ ID NO: 55)
Chr13: 6192628
5′- CCC CAA AAG TAG ACA ATT
TCC TT -3′
(SEQ ID NO: 56)
Chr14: 6685859
5′- TGG TTT GAT CAA TAG TTT CTT
AGG G -3′
(SEQ ID NO: 57)
Chr15: 6955482
5′- CCC AAG AGA GGA GGG AGT TT -
3′
(SEQ ID NO: 58)
Chr16: 5321310
5′- ATC TCT GAT CCC AGG AGG TG -
3′
(SEQ ID NO: 59)
Chr17: 3383258
5′- TGA GAA TTT CAT GCG AGA GC -
3′
(SEQ ID NO: 60)
Chr17: 32 Mbp region (H2-K)
5′- cct ggg ctt cta ccc tgc t -3′
(SEQ ID NO: 61)
or
5′- CCA CCA CAG CTC CAG TGA G -
3′
(SEQ ID NO: 62)
Ch17: 60455318
5′- CTC ACA AAA CCT GCC CTT GT -
3′
(SEQ ID NO: 63)
Chr18: 4445867
5′- GTG GGT AGC TCT GCT GAA GG -
3′
(SEQ ID NO: 64)
ChrX: 8450263
5′- AAG GAG CAG GAA CTC ATC
ACA -3′
(SEQ ID NO: 65)

TABLE 3 Legend PCR was carried out with 50 ng genomic DNA. PCR reactions were set up in a total volume of 50 μl reaction mix containing 2 units of AmpliTaq DNA polymerase (Applied Biosystems [Perkin Elmer], Weiterstadt, Germany). PCR cycling was performed using the following protocol: 94° C. for 4 min (initial denaturation); 92° C. for 40 sec, annealing 60° C. for 40° sec, 72° C. for 40° sec (35 cycles); 72° C. for 10 min (final elongation). PCR products were purified using Qiaquick PCR purification kit (Qiagen, Valencia, Calif., USA). The DNA sequence analysis with the purified PCR products was performed by the Molecular Genetics Core Facility—Boston Children's Hospital-Harvard Medical School.

### Example II

### Parthenogenesis Human Embryos p(MII)

Parthenogenesis:

Human oocytes that fail to fertilize (or fresh unfertilized oocytes) will be washed in 20 μL drops of HEPES-buffered HTF (human tubal fluid)+5% HSA (human serum albumin) and subsequently placed in a four-well culture plate of Ham F10 media with puromycin 10 μgm/ml. The oocytes will then be checked at 6 and 12 hours for the presence of a second polar body or pronucleus. If either is noted, the activated oocytes will be washed again and cultured in G1.3/G2.2 media (Vitrolife).

Alternatively the failed to fertilize oocytes after washing with HEPES-buffered HTF+5% HSA will be exposed for 5-10 minutes to 5 μM calcium ionophore in HEPES-buffered HTF+5% HSA followed by 3-6 hours incubation in 1 mM 6-DMAP (6 dimethylaminopurine). Subsequently the oocytes will be cultured in G1.3/G2.3 media. Culture is performed at 37 C in 5% CO2, 5% O2, 90% N2 (Santos T A, Dias C, Henriques P, et al. Cytogenetic analysis of spontaneously activated noninseminated oocytes and parthenogenetically activated failed fertilized human oocytes—implications for the use of primate parthenotes for stem cell production. J Assist Reprod Genet 2003; 20(3):122-30; Lin H, Lei J, Wininger D, et al. Multilineage potential of homozygous stem cells derived from metaphase II oocytes. Stem Cells 2003; 21(2):152-61).

### Example III

### Transplantation of Hematopoietic Stem Cells Derived from p(II)ES and p(I)ES Cells

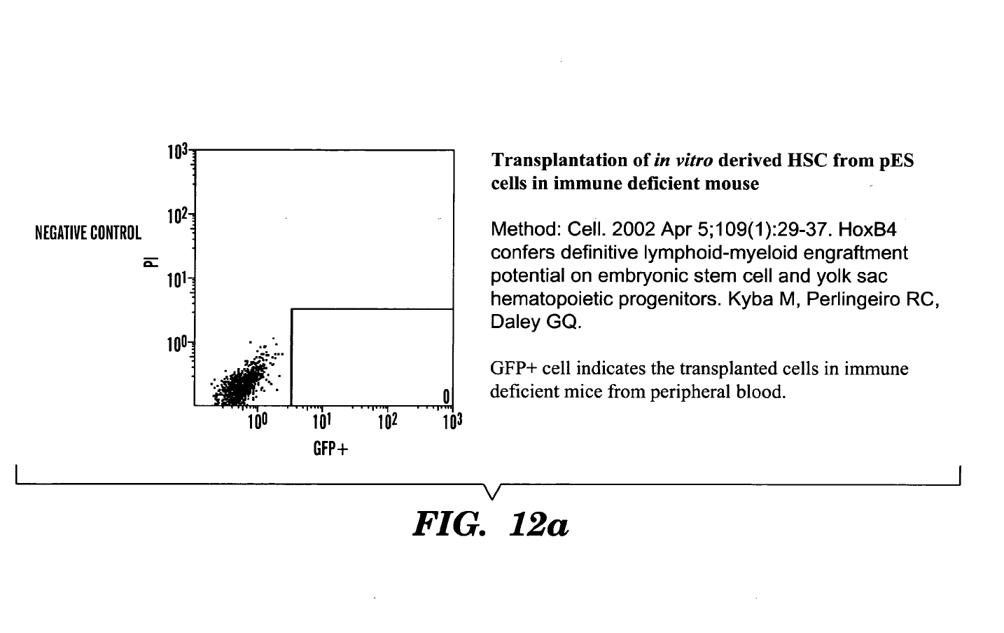

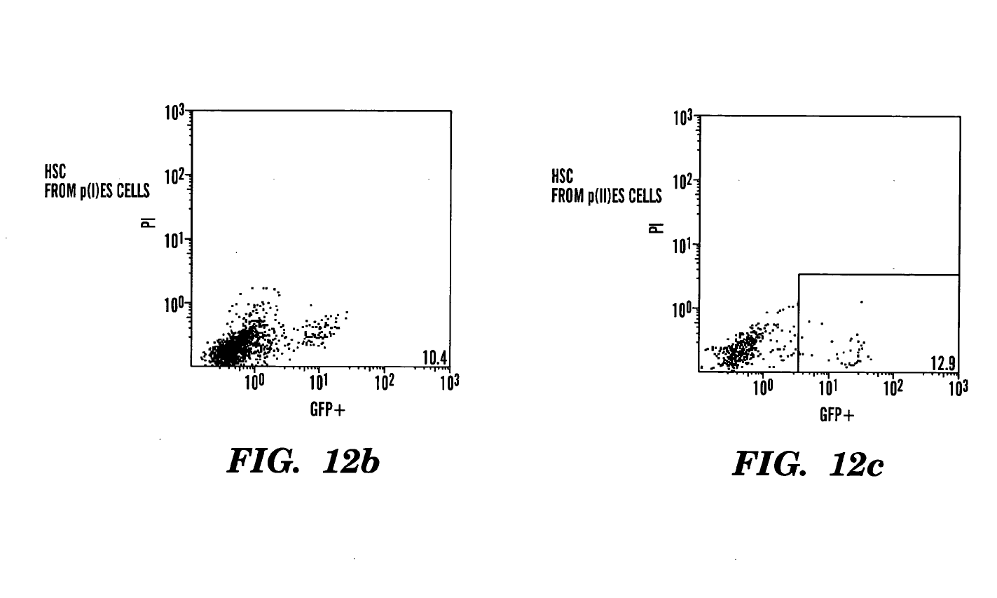

p(II)ES and p(I)ES cells expressing green fluorescent protein (GFP) were differentiated in vitro using HoxB4 protein mediated OP9 stroma cell coculture method and transplanted into immune deficient mice as indicated in Kyba et al., Cell, 2002, April 5:109(1):29-37 HoxB4 confers definitive lymphoid-myeloid engraftment potential on embryonic stem cell and yolk sac hematopoietic progenitors. Peripherial blood was isolated and analyzed using FACS analysis (FIG. 12a-12c). FACS analysis shows that hematopoietic stem cells derived from both p(II)ES (FIG. 12c) and p(I)ES cells (FIG. 12b), after transplantation, successfully reconstituted peripheral blood.

All references cited herein and throughout the Application are herein incorporated by reference.

### REFERENCES

1. N. D. Allen, S. C. Barton, K. Hilton, M. L. Norris, M. A. Surani, Development 120, 1473 (June, 1994).
2. J. B. Cibelli et al., Science 295, 819 (Feb. 1, 2002).
3. H. Lin et al., Stem Cells 21, 152 (2003).
4. W. M. Rideout, 3rd, K. Hochedlinger, M. Kyba, G. Q. Daley, R. Jaenisch, Cell 109, 17 (2002).
5. S. C. Barton, M. A. Surani, M. L. Norris, Nature 311, 374 (Sep. 27-Oct. 3, 1984).
6. L. Strain, J. P. Warner, T. Johnston, D. T. Bonthron, Nat Genet 11, 164 (October, 1995).
7. S. K. Kung, R. G. Miller, J Immunol 154, 1624 (Feb. 15, 1995).
8. T. Juji et al., N Engl J Med 321, 56 (Jul. 6, 1989).
9. K. Takahashi et al., Lancet 343, 700 (Mar. 19, 1994).
10. G. Opelz, T. Wujciak, B. Dohler, S. Scherer, J. Mytilineos, Rev Immunogenet 1, 334 (1999).
11. National-Center-for-Biotechnology-information.
12. S. H. Klebs, M. W. Hoffmann, P. B. Musholt, B. Bayer, T. J. Musholt, J Immunol Methods 276, 197 (May 1, 2003).
13. E. Y. Fok, P. W. Zandstra, Stem Cells 23, 1333 (October, 2005).
14. J. Kubiak, A. Paldi, M. Weber, B. Maro, Development 111, 763 (March, 1991).
15. The-Jackson-Laboratory, www.informatics.jax.org/(2006).
16. J. L. Moran et al., Genome Res 16, 436 (March, 2006).
17. W. F. Dietrich et al., Nature 380, 149 (Mar. 14, 1996).
18. S. Bao et al., Hum Reprod 17, 1311 (May, 2002).
19. J. R. Mann, I. Gadi, M. L. Harbison, S. J. Abbondanzo, C. L. Stewart, Cell 62, 251 (Jul. 27, 1990).
20. H. K. Mikkola, Y. Fujiwara, T. M. Schlaeger, D. Traver, S. H. Orkin, Blood 101, 508 (Jan. 15, 2003).
21. M. Zijistra et al., Nature 344, 742 (Apr. 19, 1990).
22. W. S. Hwang et al., Science (Feb. 12, 2004).
23. Seoul-National-University-Investigation-Committee, http://www.snu.ac.kr/sk_zon/sk_zon_ne/sk_zon_nea/sk_zon_nea.jsp?url=http://www.snu.ac.kr/sk_board/xxx/../boards/sk_news_read.jsp?id=66751 January (January, 2006).
24. Seoul-National-University-Investigation-Committee, http://www.snu.ac.kr/sk_zon/sk_zon_ne/sk_zon_nea/sk_zon_nea.jsp?url=http://www.snu.ac.kr/sk_{—}board/xxx/../boards/sk_news_read.jsp?id=66751 May (May, 2006).
25. M. B. Jackson et al., Pflugers Arch 417, 129 (October, 1990).
26. N. Geijsen et al., Nature 427, 148 (Jan. 8, 2004).
27. K. L. Gunderson et al., Genome Res 14, 870 (May, 2004).
28. A. Oliphant, D. L. Barker, J. R. Stuelpnagel, M. S. Chee, Biotechniques Suppl, 56 (June, 2002).
29. B. J. Yoon et al., Nat Genet 30, 92 (January, 2002).
30. K. Hochedlinger, R. Jaenisch, Nature 415, 1035 (2002).
31. Abramczuk, J. W. and A. Lopata, Resistance of human follicular oocytes to parthenogenetic activation: DNA distribution and content in oocytes maintained in vitro. Hum Reprod, 1990. 5(5): p. 578-81.
32. Winston, N., et al., Parthenogenetic activation and development of fresh and aged human oocytes. Fertil Steril, 1991. 56(5): p. 904-12.
33. Balakier, H. and R. F. Casper, Experimentally induced parthenogenetic activation of human oocytes. Hum Reprod, 1993. 8(5): p. 740-3.
34. De Sutter, P., et al., Cytogenetic analysis of human oocytes parthenogenetically activated by puromycin. J Assist Reprod Genet, 1994. 11(8): p. 382-8.
35. Taylor, A. S. and P. R. Braude, The early development and DNA content of activated human oocytes and parthenogenetic human embryos. Hum Reprod, 1994. 9(12): p. 2389-97.
36. Gook, D. A., S. M. Osborn, and W. I. Johnston, Parthenogenetic activation of human oocytes following cryopreservation using 1,2-propanediol. Hum Reprod, 1995. 10(3): p. 654-8.
37. Rinaudo, P., et al., Dissociation between intracellular calcium elevation and development of human oocytes treated with calcium ionophore. Fertil Steril, 1997. 68(6): p. 1086-92.
38. Nakagawa, K., et al., A combination of calcium ionophore and puromycin effectively produces human parthenogenones with one haploid pronucleus. Zygote, 2001. 9(1): p. 83-8.
39. Santos, T. A., et al., Cytogenetic analysis of spontaneously activated noninseminated oocytes and parthenogenetically activated failed fertilized human oocytes—implications for the use of primate parthenotes for stem cell production. J Assist Reprod Genet, 2003. 20(3): p. 122-30.
40. Lin, H., et al., Multilineage potential of homozygous stem cells derived from metaphase II oocytes. Stem Cells, 2003. 21(2): p. 152-61.
41. Rogers, N. T., et al., Phospholipase Czeta causes Ca2+ oscillations and parthenogenetic activation of human oocytes. Reproduction, 2004. 128(6): p. 697-702.
42. Mitalipov, S. M., K. D. Nusser, and D. P. Wolf, Parthenogenetic activation of rhesus monkey oocytes and reconstructed embryos. Biol Reprod, 2001. 65(1): p. 253-9.

### Example IV

### Recombination Signatures Distinguish Embryonic Stem Cells Derived by Parthenogenesis and Somatic Cell Nuclear Transfer

Using genome-wide Single Nucleotide Polymorphism (SNP) analysis, we demonstrate distinct signatures of genetic recombination that distinguish parthenogenetic ES cells from those generated by SCNT. We applied SNP analysis to the human ES cell line SCNT-hES-1, previously claimed to have been derived by SCNT, and present evidence that it represents a human parthenogenetic ES cell line. Genome-wide SNP analysis represents a means to validate the genetic provenance of an ES cell line.

### Methods

Cytogenetic and Molecular Analysis

Cytogenetic analysis was performed by the Molecular Cytogenetics Core Facility of Memorial Sloan-Kettering Cancer Center, USA. DNA Finger Printing was performed by Cell Line Genetics, USA with the Powerplex 16 kit (Promega) (Goncalves et al., 2002). HLA typing was performed by the Blood Center of Wisconsin, USA with LABtype SSO kit (One Lambda Inc.) (Colinas et al., 2000). Human SNP analysis was performed by Affymetrix USA and the Molecular Genetics Core Facility of Children's Hospital Boston & Harvard Medical School with GeneChip Human Mapping Nsp Sty Array Kit (Affymetrix) (Komura et al., 2006); mouse SNP analysis was performed at the Broad Institute NCRR Center for Genotyping and Analysis using the Illumina multiplexed allele extension and ligation method (Golden Gate) with detection using oligonucleotide probes covalently attached to beads that are assembled into fiber optic bundles (Bead Array) (Moran et al., 2006).

SNP Data Analysis

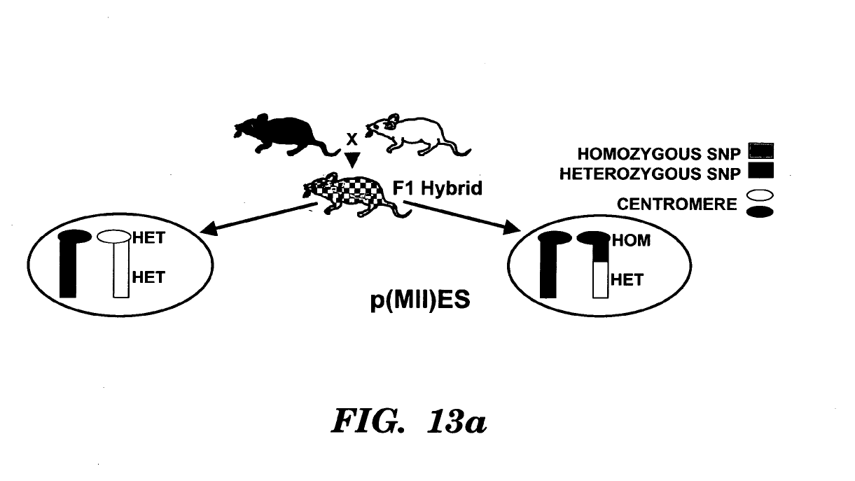

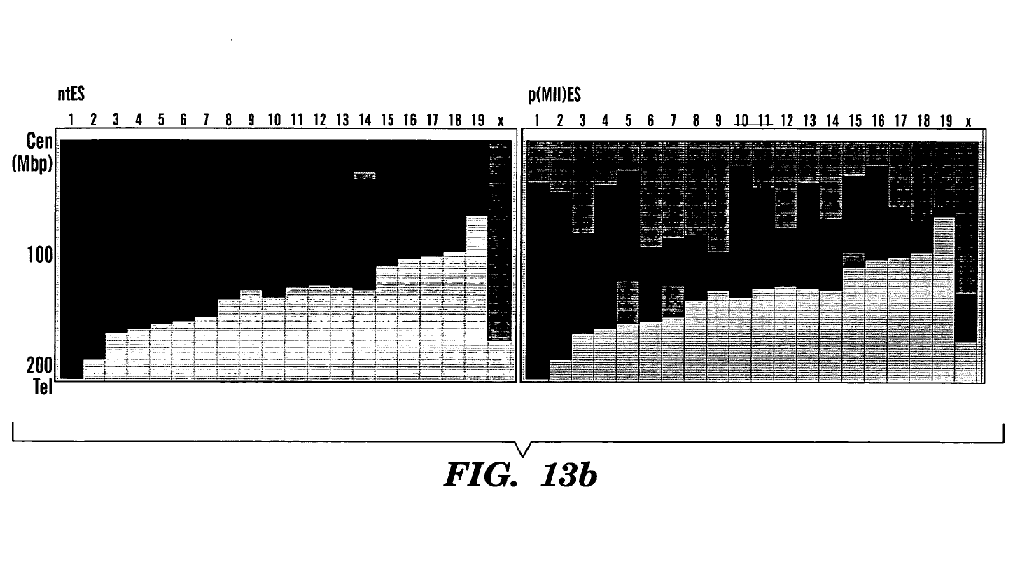

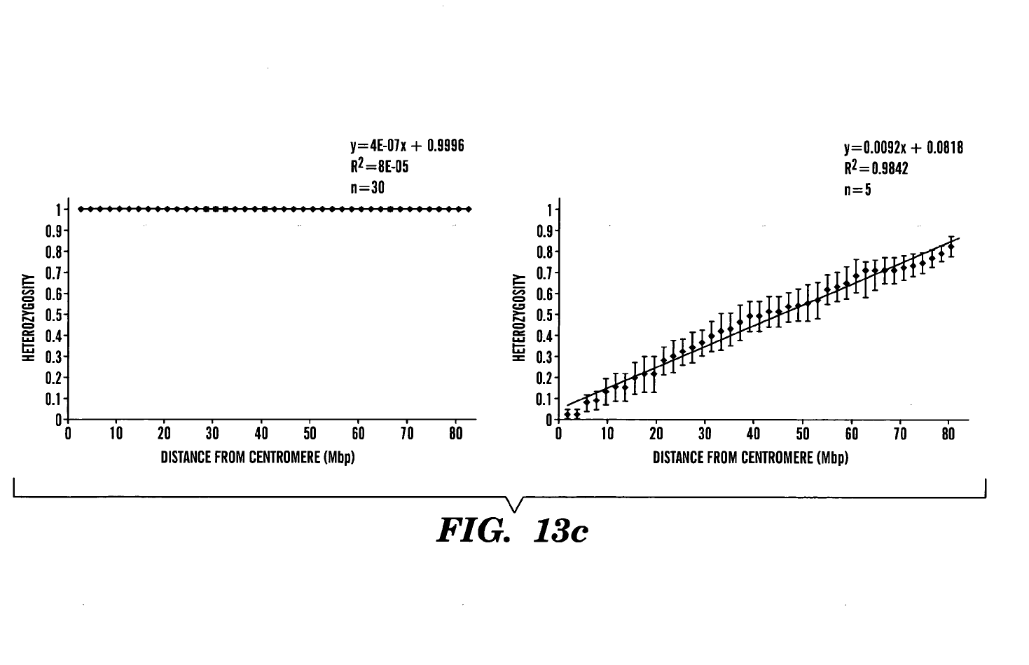

In a prior analysis of SNP data from pES cells, we pooled data for each chromosome among multiple pES cells to calculate the relationship between marker heterozygosity and distance of the marker from the centromere (Kim et al., 2007). In order to generate a meaningful comparison of the pattern of genetic recombination in a single cell line (SCNT-hES-1) with murine ntES and pES lines, we analyzed the pre-existing SNP data for 5 euploid p(MII)ES and p(MI)ES lines by pooling data for all markers at a given distance from the centromere across all chromosomes in individual cell lines (as illustrated in FIG. 13c), thereby reducing the clonal variation we observed in the prior SNP analysis (Kim et al., 2007).

Procurement of SCNT-hES-1 and Handling of Research Materials and Data:

DNA and mRNA extracts of SCNT-hES-1 and SCNT-hES-1 cell line were obtained from the Department of Theriogenology & Biotechnology, College of Veterinary Medicine, Seoul National University by Drs. Moore and Pederson under a material transfer agreement between their respective institutions and the Seoul National University. Research data but not materials were exchanged among the authors in the preparation of this manuscript.

### Results

We provide a thorough comparative analysis of 5 novel pES cells, 30 nuclear transfer derived murine ES lines, as well as SCNT-hES-1 by genome-wide SNP genotyping. We analyze the murine samples in a novel manner that facilitates comparison to a single cell line like SCNT-hES-1. Our analysis shows that the recombination pattern of SCNT-hES-1 is distinct from that of a ntES line and is consistent with its derivation from a parthenogenetic embryo. Thus, we conclude the derivation of SCNT-hES-1 represented the first reported successful isolation of human pES cells.

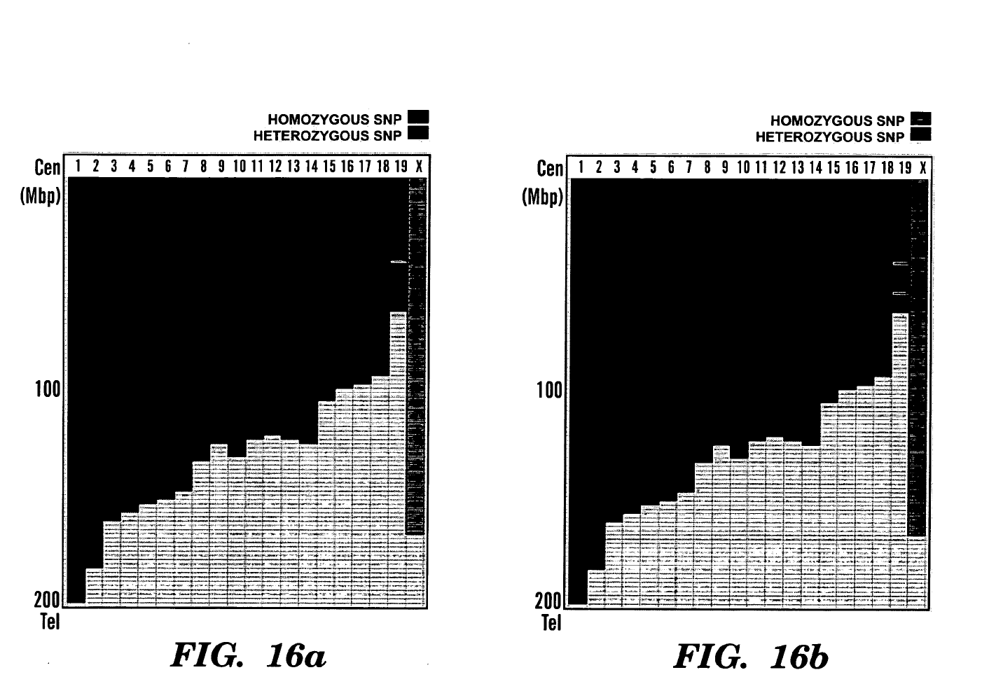

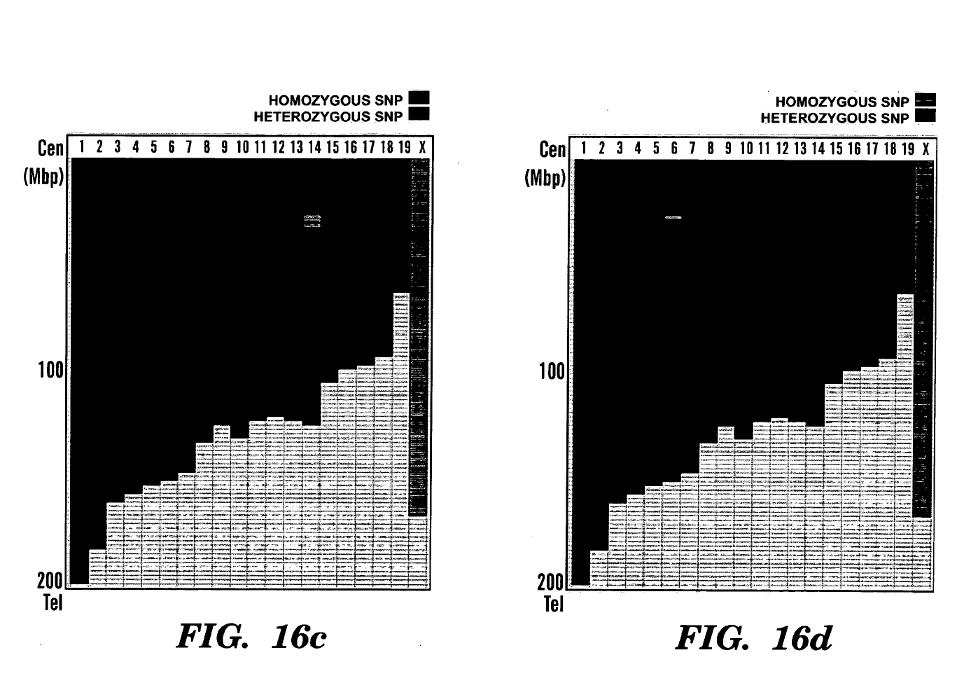

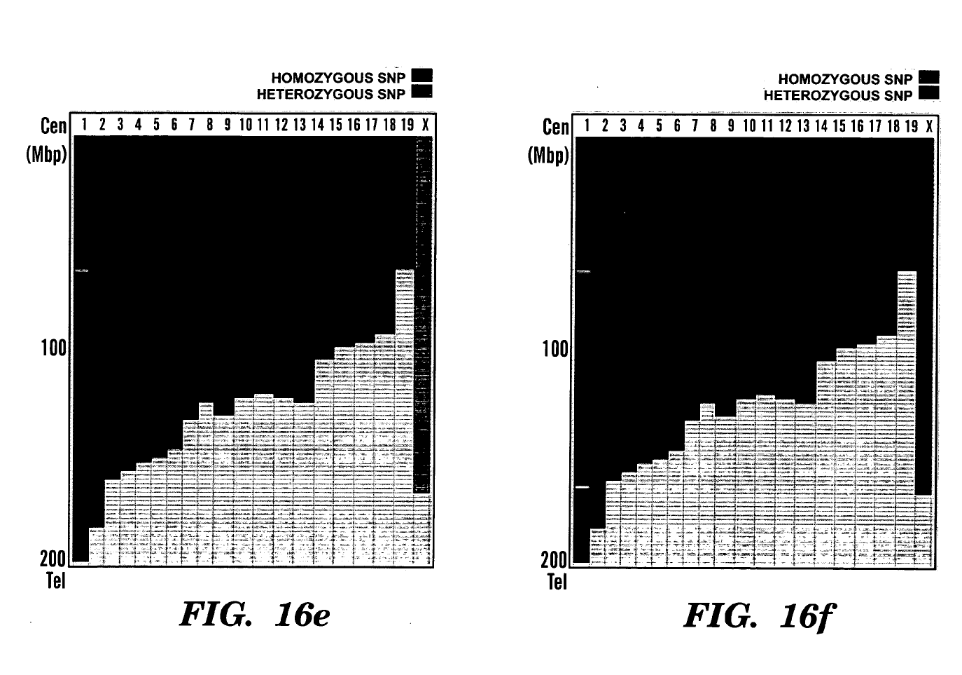

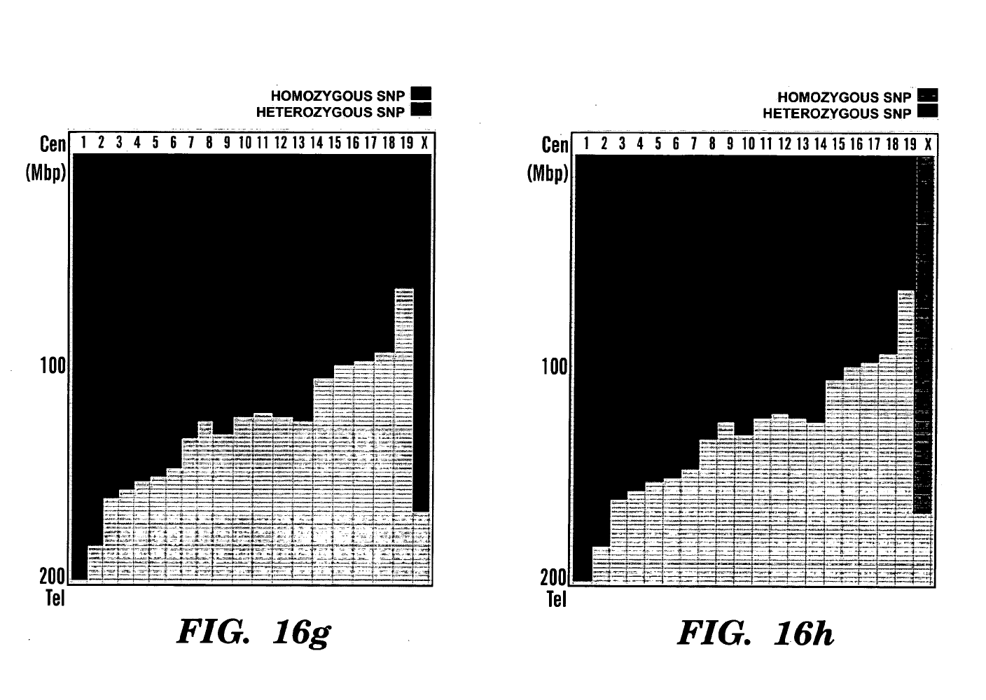

To determine the recombination patterns of ntES and pES cells, we performed genome-wide SNP analysis (Moran et al., 2006) in 30 euploid ntES cell lines generated from hybrid strains of mice using a variety of donor cells, and compared the results with 5 newly derived p(MII)ES cell lines (FIG. 13). Cell lines derived from embryos produced by nuclear transfer from a hybrid F1 mouse show complete heterozygosity at all informative SNP markers (FIG. 13b, left panels; and FIG. 16), except for rare occurrences of mitotic recombination or gene conversion (e.g., FIG. 13b chromosome 14 (Donahue et al., 2006). There is no discernable relationship between rates of marker recombination and marker distance from the centromere (FIG. 13c). Analysis of these 5 newly derived murine p(MII)ES cell lines shows the characteristic pericentromeric homozygosity (FIG. 13b, right panel) and increasing heterozygosity as marker distance increases from the centromere (FIG. 13c, right panels), which show the existence of an identical pattern regardless of genetic background (B6D2F1) and ES cell isolation method.

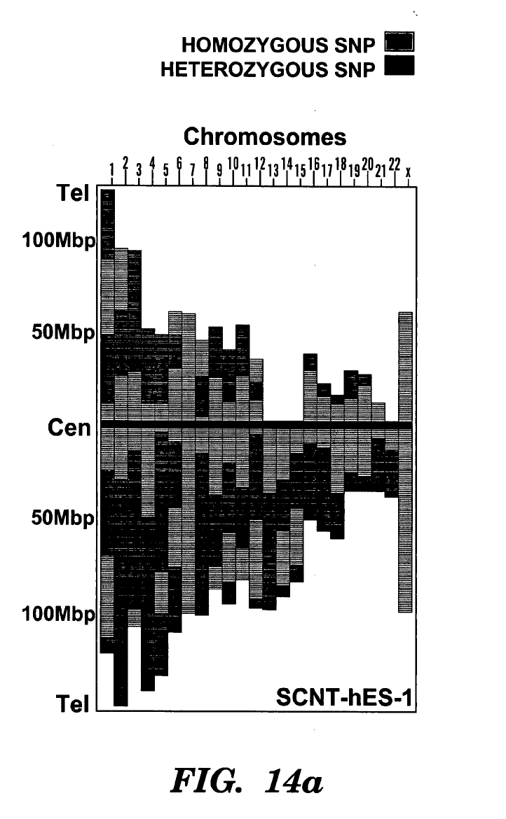

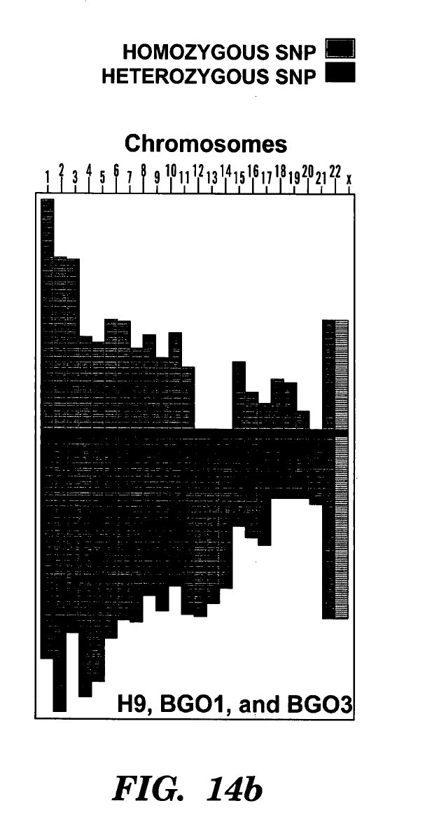

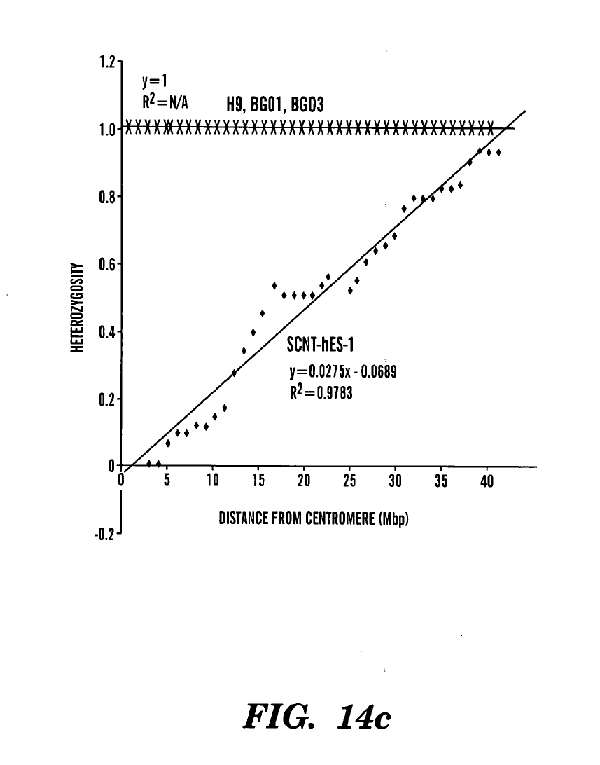

We used the GeneChip Human Mapping 500K SNP Array set (Affymetrix) to investigate the patterns of marker heterozygosity across all chromosomes of SCNT-hES-1, based on the hypothesis that derivation by SCNT would reveal genome-wide heterozygosity, whereas parthenogenesis would be reflected by large blocks of homozygosity, with the relationship of these blocks to the centromere indicative of an interruption of either meiosis I or II. For comparison, we determined the genome-wide patterns of marker heterozygosity for the human ES cell lines H9, BGO1, and BGO3, which were derived from embryos created by IVF and confirmed to have normal karyotype. Genotyping data for the hemizygous X-chromosome from the male human ES cell line, BGO1, served as a control for genotyping error rates. Across this single X-chromosome, 2.3% of genotypes were reported as heterozygous (241 out of 10,536 calls). The error rates across this chromosome fit a normal distribution, with >99% of the blocks of 1000 markers showing an error rate <5%. Thus, we assigned homozygosity to any block of 1000 SNPs (with a median distribution of one SNP per 2.5 kb) where the heterozygous SNP frequency was at or below 5.0% (50 per 1000) (footnote). Using this parameter, all of the X-chromosome regions from BGO1 fit the criteria of a homozygous chromosome, and none of the other regions in chromosomes from H9, BGO1, and BGO3 were called as homozygous regions (FIG. 14b). Differences between the heterozygous and homozygous samples were evaluated by Chi-Square analysis and revealed a high degree of significance (p<0.0001).

We analyzed the genotyping data for SCNT-hES-1 using the assumptions described above. Chromosome by chromosome, homozygosity predominates at pericentromeric markers, and heterozygosity at more distal markers (FIG. 14a). When the SNP heterozygosity data for SCNT-hES-1 is plotted with respect to the marker distance from the centromere (FIG. 14c), one observes the pattern characteristic of mouse p(MII)ES cells (FIG. 13c). This analysis suggests that SCNT-hES-1 is indeed a human p(MII)ES cell line.

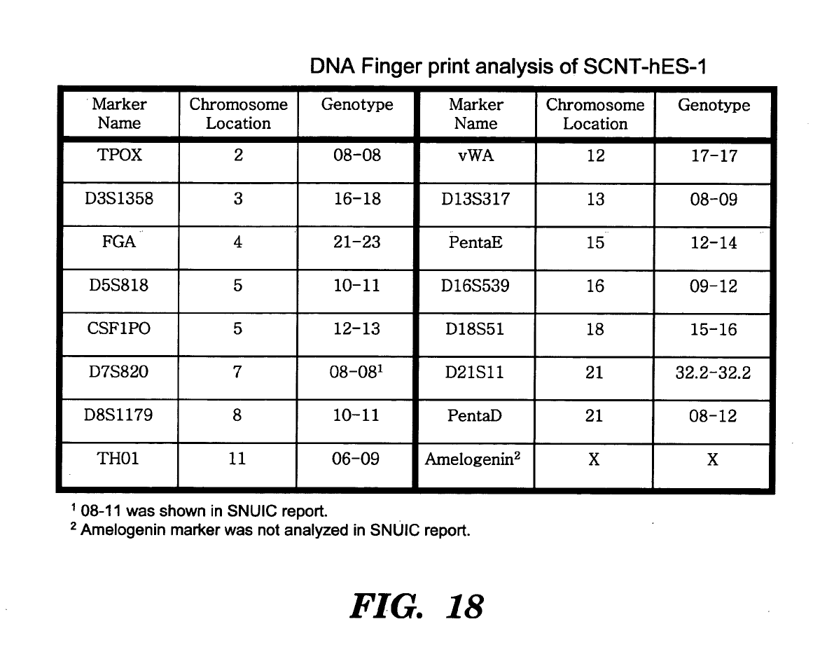

Interestingly, chromosomes 7 and X show patterns of complete homozygosity in SCNT-hES-1 (FIG. 14a). The hybridization signal for the human SNP genotyping array showed mono-allelic intensity for the X-chromosome markers and bi-allelic intensity for the markers on chromosome 7 (Komura et al., 2006). Cytogenetic analysis showed a single copy of the X-chromosome, and two copies of chromosome 7 (data not shown). The original analysis reported for SCNT-hES-1 revealed an XX karyotype, suggesting that the subline of SCNT-hES-1 cells studied here has undergone X chromosome loss. Prior DNA fingerprinting analysis of a highly polymorphic marker on chromosome 7 showed heterozygosity (D75820; 08, 11; SNUIC; (Seoul-National-University-Investigation-Committee, 2006), whereas a repeat fingerprint analysis of the subline studied here shows homozygosity (08-08), suggesting that our line sustained loss of a single copy of chromosome 7 and duplication of the remaining one, a phenomenon that has been reported in cultured cell lines (Donahue et al., 2006). Except for these differences, DNA fingerprint analysis of the subline of SCNT-hES-1 studied here using a set of 16 polymorphic markers distributed across multiple chromosomes matched the fingerprinting data reported for SCNT-hES-1 by the SNUIC (FIG. 18), thereby confirming the identity of our line of SCNT-hES-1 as the isolate reported by Hwang and colleagues.

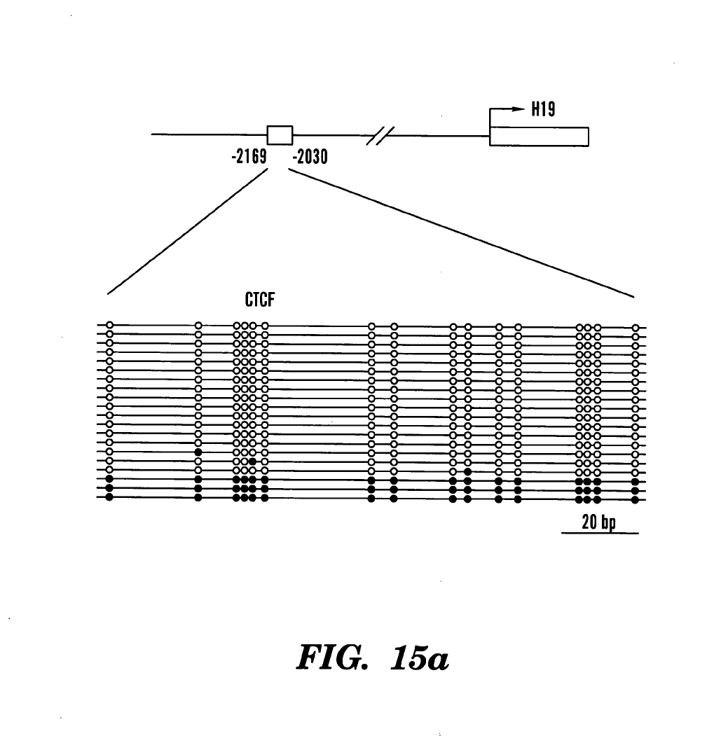

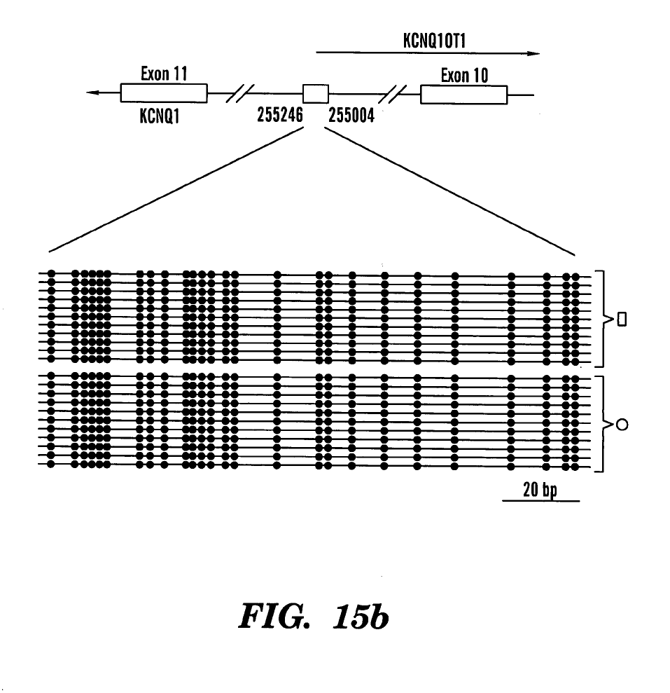

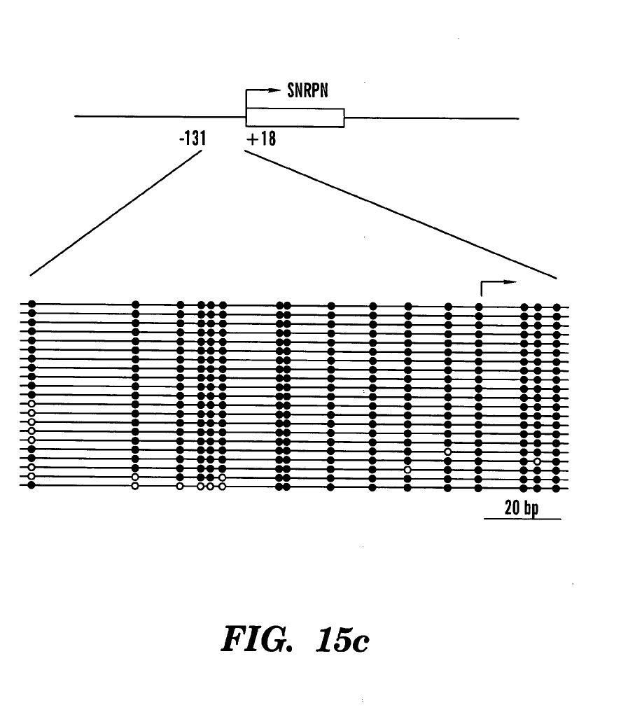

Mammalian cells carry parent-of-origin patterns of DNA methylation at imprinted gene loci due to differential modification in male and female gametes and parental-specific DNA methylation is subsequently maintained throughout development. To provide an additional assay that can distinguish parthenogenetic form biparental cell types, we analyzed the methylation status of three differentially methylated regions (DMRs) in differentiated SCNT-hES-1 cells by bisulphite treatment followed by sequencing. The normally paternally-methylated H19 DMR on chromosome 11 was predominantly unmethylated (3/20 DNA strands methylated; significantly different from the expected 10/20, p=0.002, χ2 test), whereas the normally maternally-methylated KCNQ1OT1 and SNRPN DMRs on chromosomes 11 and 15, respectively, were both fully methylated (22/22, p=3×10−6 and 21/21, p=5×10−6, respectively; FIG. 15). Importantly, a polymorphism was identified that distinguished the two KCNQ1OT1 DMR alleles, thereby revealing that both alleles were fully methylated. This epigenotype contrasts with normal differential methylation patterns observed at the same DMRs in hES cells derived from fertilized embryos (Rugg-Gunn et al., 2005a), and is characteristic of parthenogenetic cells that contain two maternal genomes and no paternal genome. This epigenetic assessment confirms our genome-wide SNP analysis, thereby providing more evidence that SCNT-hES-1 was derived from a parthenogenetically-activated embryo.

We have described a strategy for isolating murine parthenogenetic ES cells that are genetically matched to the oocyte donor at Major Histocompatibility Complex (MHC) loci (Kim et al., 2007). The mouse MI-IC cluster is located approximately 32 Mbp from the centromere on chromosome 17. This region is predicted to be 37.6% heterozygous in p(MII)ES cells (FIG. 13c) and 87.2% heterozygous in p(MI)ES cells (Kim et al., 2007). We observed MHC heterozygosity in 33% of p(MII)ES cells (24/72) and 87% of p(MI)ES cells (13/15) (Kim et al., 2007), in close agreement with our prediction.

By applying a similar analysis in human samples, we can determine the probability that any given human pES line will be genetically identical at the maternal histocompatibility loci to the oocyte donor. The recombination frequency of the human genome is higher than the mouse genome (Kong et al., 2002), and the human female genetic map is 72% larger than the male due to a higher frequency of recombination in female meiosis (Kong et al., 2002). The female human chromosome 6, which contains the human MHC cluster, has 241.55 cM of genetic distance over 190.87 Mb of physical distance (an average of 1.26 cM/Mb) (Kong et al., 2002). Thus, human chromosome 6 will reach peak heterozygosity, and thus sustain at least one cross-over, within 39.7 Mb from the centromere. The genotyping data available for SCNT-hES-1 demonstrates that peak heterozygosity is indeed reached at the predicted physical distance around 38.9 Mb from the centromere (FIG. 14c). The human MHC cluster is located 28.3-31.5 Mb from the centromere on chromosome 6. Thus, we predict that 70.9% of human p(MII)ES cells will show heterozygosity at the MHC loci and thereby match the oocyte donor in an autologous manner (FIG. 14c).

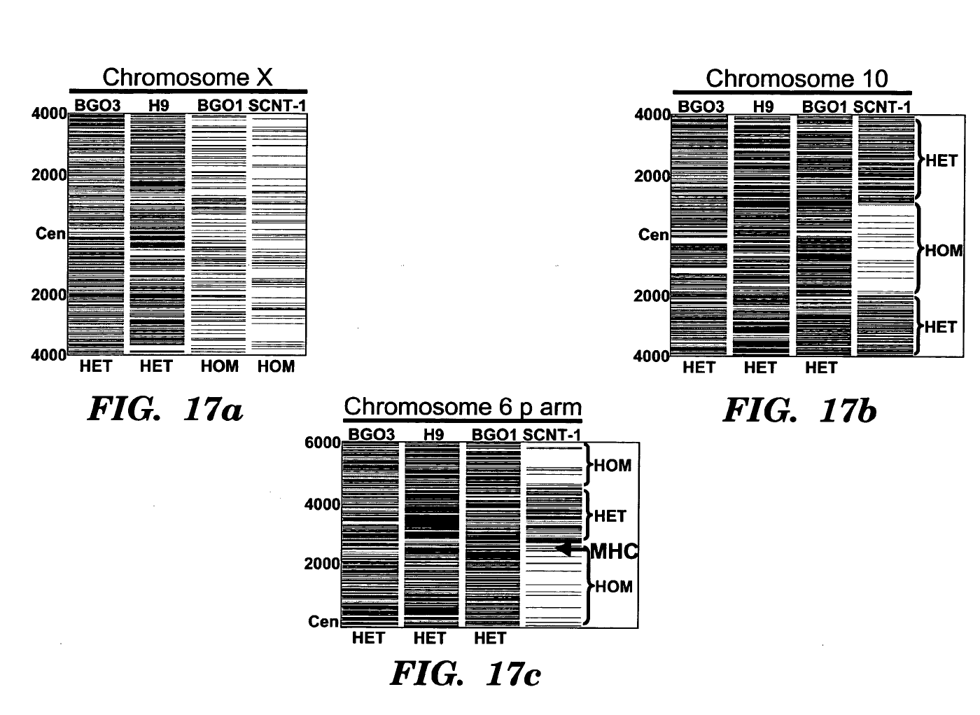

We determined the HLA type for SCNT-hES-1, and found it to be homozygous: HLA-A (31, 31), HLA-B (35, 35), HLA-Cw (03, 03), HLA-DRB1 (04, 04), and HLA-DQB1 (0302, 0302). Genetic analysis of the MHC region of SCNT-hES-1 indicates that a cross-over event occurred telomeric to the MHC-gene cluster (FIG. 17). Thus, SCNT-HES-1 represents a hemizygous HLA match to the oocyte donor.

Both parthenogenesis and nuclear transfer represent strategies for generating histocompatible ES cells for potential therapeutic use. Whereas nuclear transfer potentially provides a nearly exact match to the nuclear donor's immune identity (matching nuclear but not mitochondrial genes), parthenogenesis provides an exact match to the oocyte donor's genome (both nuclear and mitochondrial). Moreover, parthenogenesis provides a source of cells that are either heterozygous or homozygous for major histocompatibility alleles, thereby allowing either complete MHC matching to the oocyte donor, or in the case of MHC homozygosity, partial MHC matching to a substantial population of unrelated transplant recipients (Taylor et al., 2005). Parthenogenesis is a more efficient means of generating embryos and ES cell lines than nuclear transfer, and to date human nuclear transfer has not been successfully used to generate an ES cell.

During experimental parthenogenesis in the mouse, cytochalasin is added to prevent the extrusion of the second polar body and to preserve the diploid state. In contrast, in human oocytes cytochalasin is not necessary to retain diploidy (De Sutter et al., 1992; Santos et al., 2003; Taylor and Braude, 1994), and a kinase inhibitor such as 6-dimethylaminopurine (DMAP) suffices to initiate diploid parthenogenetic development (Szollosi et al., 1993). The derivation protocol of SCNT-hES-1 employed DMAP after oocyte activation with a calcium ionophore. Thus, the protocols for generating ntES lines typically involve the same steps of artificial oocyte activation as parthenogenesis, and in the case of SCNT-hES-1, there was apparently no enucleation. Alternatively, there was re-fusion of the first polar body after enucleation (Wakayama et al., 2006). Regardless of the mechanism, the result was development of a diploid parthenogenetic embryo. To rule out a parthenogenetic origin of SCNT-hES-1, Hwang and colleagues offered evidence for expression of two imprinted genes that are normally only expressed from the paternally-inherited allele. However, such aberrant expression can result from epigenetic instability, which is frequently observed in mouse pES cells (Dean et al., 1998; Feil et al., 1997). We have shown that methylation analysis of germline-acquired DMRs is a more robust indicator of epigenotype, although this too can alter following extensive in vitro culture (Humpherys et al., 2001; Mitalipov et al., 2006; Rugg-Gunn et al., 2005b).

For trials of nuclear transfer, if the somatic cell nucleus and the recipient oocytes come from different donors, the genomic DNA of any resulting ntES cells can be readily distinguished from parthenogenetic derivatives that might mistakenly arise. However, if nuclear transfer is performed using autologous oocytes from the somatic-cell donor, as in the case of SCNT-hES-1, all genetic markers will be shared, and selection of a small number of markers could mistakenly lead to the conclusion of genetic identity. Importantly, pES cells differ from ntES cells and ES cells generated from fertilized embryos in that certain regions of the genome show homozygosity and are thus only haploidentical to the oocyte donor. Genome-wide SNP genotyping is a reliable means of distinguishing parthenogenetic derivatives from those derived by nuclear transfer, because parthenogenetic embryo development incurs a diagnostic recombination signature that reflects the unique chromosomal dynamics of meiosis. Distinguishing ntES cells from those derived from fertilization embryos requires unequivocal demonstration of genetic identity to the somatic cell donor, or in cases where the somatic cell donor and oocyte donor differ, demonstration that the mitochondrial DNA is distinct from the somatic cell and instead derives from the oocyte. The evidence indicates that SCNT-hES-1 represented the first reported isolation of a human pES cell.

All references cited herein and throughout the Application are herein incorporated by reference.

### REFERENCES FOR EXAMPLE IV

Allen, N. D., Barton, S. C., Hilton, K., Norris, M. L., and Surani, M. A. (1994). A functional analysis of imprinting in parthenogenetic embryonic stem cells. Development 120, 1473-1482.
Brambrink, T., Hochedlinger, K., Bell, G. &Jaenisch, R. ES cells derived from cloned and fertilized blastocysts are transcriptionally and functionally indistinguishable. Proc Natl Acad Sci USA. 103, 933-8.Epub 2006 Jan. 17. (2006).
Blelloch, R. et al. Reprogramming efficiency following somatic cell nuclear transfer is influenced by the differentiation and methylation state of the donor nucleus. Stem Cells. 24, 2007-13.Epub 2006 May 18. (2006).
Cibelli, J. B., Grant, K. A., Chapman, K. B., Cunniff, K., Worst, T., Green, H. L., Walker, S. J., Gutin, P. H., Vilner, L., Tabar, V., et al. (2002). Parthenogenetic stem cells in nonhuman primates. Science 295, 819.
Colinas, R. J., Bellisario, R., and Pass, K. A. (2000). Multiplexed genotyping of beta-globin variants from PCR-amplified newborn blood spot DNA by hybridization with allele-specific oligodeoxynucleotides coupled to an array of fluorescent microspheres. Clin Chem 46, 996-998.
De Sutter, P., Dozortsev, D., Cieslak, J., Wolf, G., Verlinsky, Y., and Dyban, A. (1992). Parthenogenetic activation of human oocytes by puromycin. J Assist Reprod Genet 9, 328-337.
Dean, W., Bowden, L., Aitchison, A., Klose, J., Moore, T., Meneses, J. J., Reik, W., and Feil, R. (1998). Altered imprinted gene methylation and expression in completely ES cell-derived mouse fetuses: association with aberrant phenotypes. Development 125, 2273-2282.
Donahue, S. L., Lin, Q., Cao, S., and Ruley, H. E. (2006). Carcinogens induce genome-wide loss of heterozygosity in normal stem cells without persistent chromosomal instability. Proc Natl Acad Sci USA 103, 11642-11646. Epub 12006 July 11625.
Eckardt, S., Leu, N. A., Bradley, H. L., Kato, H., Bunting, K. D., and McLaughlin, K. J. (2007). Hematopoietic reconstitution with androgenetic and gynogenetic stem cells. Genes Dev 21, 409-419.
Feil, R., Boyano, M. D., Allen, N. D., and Kelsey, G. (1997). Parental chromosome-specific chromatin conformation in the imprinted U2af1-rs1 gene in the mouse. J Biol Chem 272, 20893-20900.
footnote In such an analysis, the random variable is calculated to be 49.429 in a normal distribution with an error rate of 1%, meaning that as many as 49 individual heterozygous SNP's per 1000 could occur by chance alone.
Goncalves, R., Jesus, J., Fernandes, A. T., and Brehm, A. (2002). Genetic profile of a multi-ethnic population from Guine-Bissau (west African coast) using the new PowerPlex 16 System kit. Forensic Sci Int 129, 78-80.
Hernandez, L., Kozlov, S., Piras, G., and Stewart, C. L. (2003). Paternal and maternal genomes confer opposite effects on proliferation, cell-cycle length, senescence, and tumor formation. Proc Natl Acad Sci USA 100, 13344-13349. Epub 12003 October 13327.
Humpherys, D., Eggan, K., Akutsu, H., Hochedlinger, K., Rideout, W. M., 3rd, Biniszkiewicz, D., Yanagimachi, R., and Jaenisch, R. (2001). Epigenetic instability in ES cells and cloned mice. Science 293, 95-97.
Investigation Committee Report, S. N. U. (2006). Members: Chairman Myung-Hee Chung, SNU, Uhtaek Oh, SNU, Hong-Hee Kim, SNU, Un Jong Pak, SNU, Yong Sung Lee, Hanyang University, In Won Lee, SNU, In Kwon Chung, Yonsei University, Jin Ho Chung, SNU.
Kaufman, M. H., Robertson, E. J., Handyside, A. H., and Evans, M. J. (1983). Establishment of pluripotential cell lines from haploid mouse embryos. J Embryol Exp Morphol 73, 249-261.
Kennedy, D. (2006). Editorial retraction. Science 311, 335. Epub 2006 January 2012.
Kim, K., Lerou, P., Yabuuchi, A., Lengerke, C., Ng, K., West, J., Kirby, A., Daly, M. J., and Daley, G. Q. (2007). Histocompatible embryonic stem cells by parthenogenesis. Science 315, 482-486. Epub 2006 December 2014.
Komura, D., Shen, F., Ishikawa, S., Fitch, K. R., Chen, W., Zhang, J., Liu, G., Ihara, S., Nakamura, H., Hurles, M. E., et al. (2006). Genome-wide detection of human copy number variations using high-density DNA oligonucleotide arrays. Genome Res 16, 1575-1584. Epub 2006 November 1522.
Kong, A., Gudbjartsson, D. F., Sainz, J., Jonsdottir, G. M., Gudjonsson, S. A., Richardsson, B., Sigurdardottir, S., Barnard, J., Hallbeck, B., Masson, G., et al. (2002). A high-resolution recombination map of the human genome. Nat Genet 31, 241-247. Epub 2002 June 2010.
Kono, T., Obata, Y., Wu, Q., Niwa, K., Ono, Y., Yamamoto, Y., Park, E. S., Seo, J. S., and Ogawa, H. (2004). Birth of parthenogenetic mice that can develop to adulthood. Nature 428, 860-864.
Kubiak, J., Paldi, A., Weber, M., and Maro, B. (1991). Genetically identical parthenogenetic mouse embryos produced by inhibition of the first meiotic cleavage with cytochalasin D. Development 111, 763-769.
Liu, J., Van Der Elst, J., and Dhont, M. (2003). In vitro parthenogenetic development of mouse oocytes following reciprocal transfer of the chromosome spindle between in vivo-matured oocytes and in vitro-matured oocytes. Biol Reprod 68, 186-189.
Mitalipov, S., Clepper, L., Sritanaudomchai, H., Fujimoto, A., and Wolf, D. (2006). Methylation Status of Imprinting Centers for H 19/IGF2 and SNURF/SNRPN in Primate Embryonic Stem Cells. Stem Cells 14, 14.
Moran, J. L., Bolton, A. D., Tran, P. V., Brown, A., Dwyer, N. D., Manning, D. K., Bjork, B. C., Li, C., Montgomery, K., Siepka, S. M., et al. (2006). Utilization of a whole genome SNP panel for efficient genetic mapping in the mouse. Genome Res 16, 436-440.
Paffoni, A., Brevini, T. A., Somigliana, E., Restelli, L., Gandolfi, F., and Ragni, G. (2007). In vitro development of human oocytes after parthenogenetic activation or intracytoplasmic sperm injection. Fertil Steril 87, 77-82. Epub 2006 October 2030.
Revazova, E. S., Turovets, N. A., Kochetkova, O. D., Kindarova, L. B., Kuzmichev, L. N., Janus, J. D., and Pryzhkova, M. V. (2007) Patient-Specific Stem Cell Lines Derived from Human Parthenogenetic Blastocysts. Cloning and Stem Cells 9, DOI: 10.1089/clo.2007.0033
Rideout, W. M., 3rd, Hochedlinger, K., Kyba, M., Daley, G. Q., and Jaenisch, R. (2002). Correction of a genetic defect by nuclear transplantation and combined cell and gene therapy. Cell 109, 17-27.
Robertson, E. J., Evans, M. J., and Kaufman, M. H. (1983). X-chromosome instability in pluripotential stem cell lines derived from parthenogenetic embryos. J Embryol Exp Morphol 74, 297-309.
Rugg-Gunn, P. J., Ferguson-Smith, A. C., and Pedersen, R. A. (2005a). Epigenetic status of human embryonic stem cells. Nat Genet 37, 585-587. Epub 2005 May 2001.
Rugg-Gunn, P. J., Ferguson-Smith, A. C., and Pedersen, R. A. (2005b). Human embryonic stem cells as a model for studying epigenetic regulation during early development. Cell Cycle 4, 1323-1326. Epub 2005 October 1322.
Santos, T. A., Dias, C., Henriques, P., Brito, R., Barbosa, A., Regateiro, F., and Santos, A. A. (2003). Cytogenetic analysis of spontaneously activated noninseminated oocytes and parthenogenetically activated failed fertilized human oocytes—implications for the use of primate parthenotes for stem cell production. J Assist Reprod Genet 20, 122-130.
Seoul-National-University-Investigation-Committee (2006). Final Report on Professor Woo Suk Hwang's Research Allegations. http://wwwsnuackr/sk_zon/sk_zon_ne/sk_zon_nea/sk_zon_neajsp?url=http://wwwsnuac kr/sk_board/xxx//boards/sk_news_readjsp?id=66751 January.
Strain, L., Warner, J. P., Johnston, T., and Bonthron, D. T. (1995). A human parthenogenetic chimaera. Nat Genet 11, 164-169.
Szollosi, M. S., Kubiak, J. Z., Debey, P., de Pennart, H., Szollosi, D., and Maro, B. (1993). Inhibition of protein kinases by 6-dimethylaminopurine accelerates the transition to interphase in activated mouse oocytes. J Cell Sci 104, 861-872.
Taylor, A. S., and Braude, P. R. (1994). The early development and DNA content of activated human oocytes and parthenogenetic human embryos. Hum Reprod 9, 2389-2397.
Taylor, C. J., Bolton, E. M., Pocock, S., Sharples, L. D., Pedersen, R. A., and Bradley, J. A. (2005). Banking on human embryonic stem cells: estimating the number of donor cell lines needed for HLA matching. Lancet 366, 2019-2025.
Wakayama, S. et al. Establishment of male and female nuclear transfer embryonic stem cell lines from different mouse strains and tissues. Biol Reprod. 72, 932-6.Epub 2004 Dec. 15. (2005).
Wakayama, S., Hikichi, T., Suetsugu, R., Sakaide, Y., Bui, H. T., Mizutani, E., and Wakayama, T. (2006). Efficient establishment of mouse embryonic stem cell lines from single blastomeres and polar bodies. Stem Cells 21, 21.
Wu, Q., Kumagai, T., Kawahara, M., Ogawa, H., Hiura, H., Obata, Y., Takano, R., and Kono, T. (2006). Regulated expression of two sets of paternally imprinted genes is necessary for mouse parthenogenetic development to term. Reproduction 131, 481-488.

1. A method for producing a heterozygous embryonic stem (ES) cell line comprising:
a. obtaining a diploid oocyte that is in prophase or metaphase I of meiosis I, wherein the diploid oocyte comprises DNA derived from a single individual male or female;
b. culturing the oocyte under conditions that inhibit formation of the first polar body such that the cell remains diploid;
c. activating the oocyte of step (b) to induce parthenogenetic development;
d. culturing said activated oocyte to produce an embryo comprising a discernible trophectoderm and an inner cell mass;
e. isolating said inner cell mass, or cells therefrom, and transferring said inner cell mass, or cells, to an in vitro media that inhibits differentiation of said inner cell mass or cells derived therefrom; and
f. culturing said inner cell mass cells, or cells derived therefrom, to maintain said cells in an undifferentiated state thereby generating an embryonic stem cell line that is substantially heterozygous.

2. The method of claim 1, wherein step (f) comprises maintaining the cells in a pluripotent state.

3. The method of claim 1, further comprising step (g) that comprises analyzing the cells of step (f) for heterozygosity at a desired locus and selecting cells that are heterozygous at said desired locus.

4. The method of claim 3, wherein said DNA derived from a single individual male or female is human DNA, said desired locus is a Human Leukocyte Antigen (HLA) locus and wherein cells that are heterozygous for at least one HLA locus are selected.

5. The method of claim 4, further comprising the step of analyzing the cells that are heterozygous for at least one HLA locus for diploid or tetraploid DNA content.

6. The method of claim 5, wherein the embryonic stem cells that have diploid DNA content are selected and maintained in a pluripotent state.

7. The method of claim 5, wherein the embryonic stem cells that have tetraploid DNA content are selected and maintained in a pluripotent state.

8. The method of claim 4, wherein the HLA locus is selected from the group consisting of: HLA-A, HLA-B, HLA-C, HLA-DR, HLA-DQ, and HLA-DP.

9. The method of claim 4, wherein the cells that are heterozygous for at least one HLA locus are heterozygous at each of the following HLA loci: HLA-A, HLA-B, HLA-C, HLA-DR, HLA-DQ, and HLA-DP.

10. The method of claim 1, wherein the diploid oocyte is a human, non-human primate, murine, bovine, porcine, or ovine.

11. The method of claim 1, wherein the diploid DNA derived from a single individual is human, bovine, primate, murine, ovine, or porcine.

12. The method of claim 1, wherein the diploid ocyte is gynogenetically produced.

13. The method of claim 1, wherein diploid ocyte is androgenetically produced.

14. The method of claim 1, wherein the conditions that inhibit formation of the first polar body include incubation of said oocyte with cytochalasin D.

15. The method of claim 1, wherein the diploid cells are human oocytes containing human male or human female DNA.

16. The method of claim 1, wherein said cultured cells of (f) are allowed to differentiate.

17. The method of claim 1, wherein said cells of (f) are implanted at a desired site in vivo that is to be engrafted with cells or tissue.

18. The method of claim 14, wherein said cells are implanted in an immunocompromised non-human animal.

19. The method of claim 15, wherein said site is a wound, a joint, muscle, bone, or the central nervous system.

20. The method of claim 1, wherein the cell obtained by (f) is genetically modified.

21. A stem cell bank comprising a library or plurality of human or non-human animal embryonic stem cell lines generated by the method of claim 1.

22. A method for producing stem cells that are heterozygous for at least one MHC locus comprising:
a. obtaining oocyte cells in metaphase II that comprises haploid DNA derived from a single individual male or female, which optionally may be genetically modified;
b. activating the oocyte cells of step (b) to induce parthenogenetic development under conditions that inhibit second polar body formation;
c. culturing said activated oocytes to produce an embryos comprising a discernible trophectoderm and an inner cell mass;
d. isolating said inner cell mass, or cells therefrom, and transferring said inner cell mass, or cells, to an in vitro media that inhibits differentiation of said inner cell mass or cells derived therefrom thereby generating pluripotent embryonic stem (pES) cell lines; and
e. selecting pES cell lines that have undergone recombination at least one MHC locus; and
f. culturing the pES cells of step (e) to maintain said cells in an undifferentiated state thereby generating a pES cell line that is heterozygous for at least one MHC locus.

23. The method of claim 22, wherein said pES cell line of step (f) that is heterozygous for at least one MHC locus comprises human DNA and is heterozygous at a Human Leukocyte Antigen (HLA) locus selected from the group consisting of HLA-A, HLA-B, HLA-C, HLA-DR, HLA-DQ, and HLA-DP.

24. The method of claim 22, wherein said pES cell line is heterozygous at each of the following Human Leukocyte Antigen (HLA) loci: HLA-A, HLA-B, HLA-C, HLA-DR, HLA-DQ, and HLA-DP.

25. The method of claim 22, wherein step (f) comprises maintaining the cells in a pluripotent state.

26. The method of claim 22, further comprising the step of analyzing the cells of step (f) for diploid or tetraploid DNA content.

27. The method of claim 26, wherein the embryonic stem cells that have diploid DNA content are selected and maintained in a pluripotent state.

28. The method of claim 26, wherein the embryonic stem cells that have tetraploid DNA content are selected and maintained in a pluripotent state.

29. The method of claim 22, wherein the oocyte cells are human, non-human primate, murine, bovine, porcine, or ovine.

30. The method of claim 22, wherein the DNA derived from a single individual is human, bovine, primate, murine, ovine, or porcine.

31. The method of claim 22, wherein the oocyte cells in metaphase II are gynogenetically or androgenetically produced.

32. The method of claim 22, wherein the conditions that inhibit formation of the second polar body comprise incubation of said oocyte with cytochalasin B.

33. The method of claim 22, wherein the oocytes are human oocytes comprising human male or human female DNA.

34. The method of claim 22, wherein said cultured cells of (f) are allowed to differentiate.

35. The method of claim 22, wherein said cells of (f) are implanted at a desired site in vivo that is to be engrafted with cells or tissue.

36. The method of claim 35, wherein said cells are implanted in an immunocompromised non-human animal.

37. The method of claim 36, wherein said site is a wound, a joint, muscle, bone, or the central nervous system.

38. The method of claim 22, wherein the cells obtained by (f) are genetically modified.

39. A stem cell bank comprising a library or plurality of human or non-human animal embryonic stem cell lines generated by the method of claim 22.

40. The method of claim 16 or 34, wherein the cultured cells are differentiated into hematopoietic stem cells.

41. A method for determining if an embryonic stem cell line was derived from either i) a parthenogenesis embryo wherein first polor body formation was inhibited (a (pMI)ES cell line), ii) a parthenogenesis embryo wherein second polor body formation was inhibited (a (pMII)ES cell line), iii) a nuclear transfer embryo (a ntES cell line), or iv) a natural fertilization embryo comprising the steps of:
a. genotyping the cells for heterozygosity using heterozygous SNP markers
b. plotting the heterozygous rate (heterozygous SNP markers/total SNP makers) versus SNP marker distance from centromere on a graph wherein the X axis is the heterozygous rate and the Y axis is the SNP marker distance from centromere; and
c. obtaining a slope from the graph of step b wherein a negative slope in step (c) indicates a p(MI)ES cell line; a positive slope in step (c) indicates a p(MII)ES cell line; and no discernable slope in step (c) indicates a ntES cell line or a cell line derived from a natural fertilization embryo.
